(19) United States
(12) Patent Application Publication
(10) Pub. No.: US 2012/0245439 A1
(43) Pub. Date: Sep. 27, 2012

(54) METHOD AND APPARATUS FOR DETERMINING CRITICAL CARE PARAMETERS

(76) Inventors: David Andre, San Francisco, CA (US); Kevin Ward, Glen Allen, CA (US); Nisang Vyas, Bopal Ahmedabad Gujarat (IN); Eric Teller, San Francisco, CA (US); John M. Stivoric, Pittsburgh, PA (US); Jonathan Farringdon, Pittsburgh, PA (US); Scott K. Boehmke, Wexford, PA (US); Christopher Pacione, Pittsburgh, PA (US); Raymond Pelletier, Pittsburgh, PA (US); Kevin Ross, Pittsburgh, PA (US); Scott Safier, New York, NY (US); Suresh Vishnubhatla, Wexford, PA (US); Gregory Kovacs, Palo Alto, CA (US); James Gasbarro, Pittsburgh, PA (US); Christopher Kasabach, Pittsburgh, PA (US)

(21) Appl. No.: 13/130,282

(22) PCT Filed: Nov. 20, 2009
(86) PCT No.: PCT/US09/06234

§ 371 Date: Jun. 18, 2012

Related U.S. Application Data
(60) Provisional application No. 61/116,364, filed on Nov. 20, 2008.

(51) Int. Cl. A61B 5/00; A61B 5/0402; A61B 5/08; A61B 5/01; A61B 5/053; A61B 5/0488; A61B 5/1455; A61B 5/11; A61B 5/0476; A61B 5/0496; A61B 5/021; A61B 5/103; A61B 5/145; A61B 6/00; A61B 5/085; A61B 5/1468; A61B 5/024

(52) U.S. Cl. 600/310; 600/300; 600/508; 600/509; 600/529; 600/549; 600/547; 600/546; 600/595; 600/544; 600/558; 600/485; 600/587; 600/364; 600/365; 600/407; 600/538; 600/345; 600/476

(57) ABSTRACT

A physiological measuring system is disclosed that monitors certain physiological parameters of an individual through the use of a body-mounted sensing apparatus. The apparatus is particularly adapted for continuous wear. The system is also adaptable or applicable to calculating derivations of such parameters. A oxygen debt measuring embodiment is directed predicting an outcome in response to injury and illness. The technique allows for closed-loop resuscitation, early identification of illness and early corrective action.

### CROSS REFERENCE TO RELATED APPLICATIONS

This application claims priority via 35 U.S.C. 371 to International Application No. PCT/US09/06234 filed on Nov. 20, 2009. International Application No. PCT/US09/06234 is a continuation-in-part of U.S. application Ser. No. 11/928,302, filed on Oct. 30, 2007, which is a continuation of U.S. application Ser. No. 10/940,889, filed Sep. 13, 2004, issued as U.S. Pat. No. 7,502,643, which claims the benefit of U.S. Provisional Application Ser. No. 60/502,764, filed Sep. 12, 2003; U.S. Provisional Application Ser. No. 60/510,013, filed Oct. 9, 2003; and U.S. Provisional Application Ser. No. 60/555,280, filed Mar. 22, 2004. International Application No. PCT/US09/06234 is also a continuation-in-part of co-pending U.S. patent application Ser. No. 10/940,214, filed Sep. 13, 2004, which is a continuation in part of co-pending U.S. application Ser. No. 10/638,588, filed Aug. 11, 2003, which is a continuation of U.S. application Ser. No. 09/602,537, filed Jun. 23, 2000, issued as U.S. Pat. No. 6,605,038, which is a continuation-in-part of co-pending U.S. application Ser. No. 09/595,660, filed Jun. 16, 2000, issued as U.S. Pat. No. 7,689,437. U.S. patent application Ser. No. 10/940,214 also claims the benefit of U.S. Provisional Application No. 60/502,764 filed on Sep. 13, 2003 and U.S. Provisional Application No. 60/555,280 filed on Mar. 22, 2004. International Application No. PCT/US09/06234 is also a continuation-in-part of U.S. patent application Ser. No. 10/682,293, filed Oct. 9, 2003, which claims the benefit of U.S. Provisional Application No. 60/417,163 filed on Oct. 9, 2002. International Application No. PCT/US09/06234 claims the benefit of U.S. Provisional Application No. 61/116,364, filed on Nov. 20, 2008. Each patent applications referenced above is incorporated herein by reference in its entirety.

### STATEMENT REGARDING FEDERALLY SPONSORED RESEARCH

This invention was made with Government support under contract Department of Defense Grant PR023081. The Government may certain rights in the invention.

### FIELD OF THE INVENTION

The present invention relates to a physiological measuring system. More specifically, the system may be used for the real-time monitoring, analysis and reporting of physiological measurements to determine a critical care parameter. Such methods could specifically be used in determining oxygen debt by continuous or semi continuous physiologic and/or mechanical measure(s) and/or other hemodynamic related parameters.

### BACKGROUND OF THE INVENTION

Trauma continues to be the leading cause of death in the United States for people between the ages of 1 and 44. Hemorrhagic shock is responsible for more than 40% of these deaths. In the combat setting an even higher number of deaths, 50% or greater, are due to hemorrhage. Due to delayed access to definitive care and more complex wounding patterns, warfighters have a higher mortality for shock compared to the civilian setting for what may be similar levels of hemorrhage. In fact, 90% of deaths of warfighter occur before provision of effective combat casualty care.

Emergency situations, such as mass casualties or the battlefield environment may limit medical personnel to use crude measures of blood loss such as mental status, heart rate, pulse quality, capillary refill, and occasionally blood pressure and pulse oximetry to determine the severity of hemorrhage and to guide treatment. When these physiological variables are abnormal, medics are prompted to aggressively resuscitate victims. However changes in the variables above occur late in hemorrhage and reflect a state of decompensation. Furthermore, this information is currently only accessible on-site and through manual means at the time of arrival of medical help after the injury. All data that may be important to decision making including data prior to injury and data after injury but prior to manual assessment is currently not available. Injuries that include traumatic brain injury resulting in unconsciousness along with environmental factors such as extreme heat or cold, and skin pigmentation of the various races, make the use of mental status, capillary refill and observation of skin pallor even more difficult to use in gauging the severity of injury or response to treatment. Pain and stress may decrease the value of heart rate monitoring. Thus the ability to intervene early prior to a state of decompensation is limited, as is the ability of the medic to effectively triage and treat multiple casualties and allocate resources effectively. Diagnostic technologies developed with an understanding of these issues may save lives on both the battlefield as well as in the civilian trauma setting.

In a noninjured, non-septic, healthy state, oxygen consumption (VO₂) is a closely regulated process because oxygen serves as the critical carbon acceptor in the generation of energy from a wide variety of metabolic fuels. Post-traumatic hemorrhage leads to a hypovolemia in which blood flow and consequently oxygen delivery to vital organs are decreased. When oxygen delivery is decreased to a degree sufficient to reduce VO₂ to below a critical level, a state of shock occurs, producing ischemic metabolic insufficiency. This degree of restriction in VO₂ can also be produced by cardiogenic or vasodilatory shock, in which oxygen delivery is restricted by low flow. When this critical level of oxygen restriction is reached, an oxygen debt or OD occurs. OD is a quantitative measure of ischemia. Specifically, it is the degree to which an organism as a whole consumes oxygen in a manner directly proportional to the delivery of oxygen available to it. The presence and extent of an OD is further highlighted by an increase in the unmetabolized metabolic acids generated by the anaerobic processes. It is the close congruence of OD and related metabolic acidemia that permits precise quantification of the severity of the ischemic shock process in both animals and humans.

The identification of both occult and inadequately resuscitated shock in critically ill and injured patients continues to be a major clinical problem. Occult shock—that is, shock that is not immediately clinically apparent—is of particular concern in the care of elderly trauma patients, who may be in early sepsis, and are frequently characterized by multiple comorbidities and/or medications that may mask the conventional signs and symptoms of shock, and wounded warfighters where diagnostic and treatment resources are limited. Shock occurring in even the relatively young and healthy victim of blunt trauma—the classical trauma patient—may be difficult to recognize because of occult hemorrhage occurring in the thorax, abdomen, retroperitoneum, pelvis, or soft tissue.

Most resuscitation strategies appear to be heavily weighted towards efforts to restore normal oxygen delivery to the tissues. It is suggested that all these efforts have lost sight of the major physiological underpinnings of the shock state. More useful would be a return to three fundamental physiological principles underlying shock and shock treatment:

(1) prevention of further oxygen debt accumulation,

(2) repayment of oxygen debt,

(3) minimization of the time to oxygen debt resolution.

Shock is a state of hypoperfusion at the cellular level that occurs when the delivery of oxygen or DO₂ to the tissues falls below the tissue oxygen consumption or VO₂ requirements, and thus represents an imbalance or mismatch between tissue DO₂ and VO₂. Oxygen delivery is dependent on blood flow, traditionally assessed globally by cardiac output, and arterial oxygen content. Clinically, multiple organ dysfunction is associated with a persistent inadequate balance of DO₂ and VO₂ of specific tissue or organ beds. Conventionally, perfusion status is assessed by whole-body endpoints such as mental status and the standard cardiovascular parameters of heart rate, palpable pulses, and systemic blood pressure. However, data from both animal models and clinical studies indicate that these measures are very poorly correlated with perfusion of specific tissue beds. Thus organ beds may have inadequate DO₂ even if gross systemic hypotension has been corrected. As a result, even if the subject is normotensive, unequal distribution of DO₂ to various tissue beds may result in isolated organ ischemia before the occurrence of whole-body ischemia. The gut in particular appears to be especially susceptible to ischemic injury; there is increasing evidence to suggest that ischemic changes in the gut drive the systemic activation of inflammatory cascades. Continuing systemic hypoperfusion has been implicated in ischemic cellular injury and cell death, which, unless corrected, leads to Systemic Inflammatory Response Syndrome, or SIRS, and irreversible Multiple Organ Dysfunction Syndrome, or MODS. Although the total incidence of MODS has decreased over the last several decades, MODS remains a leading cause of late morbidity and mortality in trauma and the mortality rate still remains high at 50-80%.

The concept of oxygen debt has been known since the early 1960's, but has not been applied uniformly in the clinical setting. OD has been shown to be the only physiological variable that can quantitatively predict survival and the development of multiple organ failure following hemorrhage. Implicit in the concept of oxygen debt is that the probability that multiple organ dysfunction and death are influenced primarily by the accumulated debt. Early animal experiments indicated that there was a minimum threshold of oxygen debt below which all animals survived, and above which mortality increased until a universally lethal threshold of debt was attained. Subsequent animal and clinical studies showed that increasing probability of mortality was directly associated with total oxygen debt, and this debt could be estimated from key metabolic markers, namely base deficit and lactate. It follows that if resuscitation is initiated before a clinically significant oxygen debt is incurred and the debt is then repaid, cellular damage will be slight or non-existent. Conversely, the likelihood of cellular damage and subsequent organ failure is substantially increased if the period of increased oxygen debt is prolonged and/or resuscitation is inadequate, i.e. failure to repay oxygen debt. Therefore, evidence of shock resolution should consist, at a minimum, of the complete repayment of oxygen debt.

Unfortunately, none of the original oxygen debt studies made any assumptions as to the time frame within which accumulated debt is to be “forgiven” or repaid. In theory, morbidity and/or mortality should not be affected by the repayment schedule, as long as no more debt is allowed to accumulate. However, in practice it is likely that debt repayment will be slower when lower volumes of resuscitation fluid are administered, or if there is a delay in the onset of definitive resuscitation. It has been observed that prolonged hemorrhagic shock coupled with inadequate resuscitation causes a relatively small proportion of immediate deaths, but nevertheless accounts for over a quarter of hospital deaths, primarily from organ failure. This certainly would have profound implications for warfighters, as traumatic brain injury is the signature injury of the current military conflicts in Iraq and Afghanistan. The recent push towards both low-volume, hypotensive and delayed resuscitation in the pre-hospital environment means that it is even more important that we re-evaluate these resuscitation strategies in terms of debt repayment schedule.

Oxygen debt can be quantitated by measuring the difference in oxygen consumption from baseline over time. Both mortality and morbidity can be predicted by quantitating the level of oxygen debt. Despite the known predictive value of this measure since the late 1950's, the determination of OD is cumbersome, expensive, and difficult via the use of indirect calorimetry or the indirect Fick method.

Since glycolysis is the predominant energy producing process during anerobiasis, its major by-product, lactate, is greatly increased. Clinicians have used lactate for many years to assess the degree of tissue hypoxia that occurs in shock states such as hemorrhagic, cardiogenie, and septic shock. Indeed, the combination of the magnitude of lactate elevation and the length of time lactate is abnormally elevated have also been demonstrated to be predictive of mortality and morbidity. Laboratory studies on animals undergoing hemorrhage have demonstrated that interval lactate measurements using traditional sampling methods can be used to semi-quantitate OD when these values are subjected to analysis techniques such as logistic regression.

However, no one to our knowledge has suggested the use of continuous or semicontinuous lactate sampling to create high-fidelity, high-precision measures of OD that can be used to replace the classic measures of OD such as indirect calorimetry and indirect-Fick methods. Nor has it been suggested that determination of OD by this method be used as a guide to treatment and resource allocation or as a method of triage or medical/surgical management of diseases resulting in the imbalance between oxygen delivery and utilization.

OD and its metabolic correlates are important quantifiers of the severity of hemorrhagic and post-traumatic shock and may serve as useful guides in the treatment of these conditions. Such guides include the examination of metabolic oxygen debt correlates, namely base deficit and lactate, as indices of shock severity and adequacy of volume resuscitation. Research suggests that oxygen debt or its metabolic correlates may be more useful quantifiers of hemorrhagic shock than estimates of blood loss, volume replacement, blood pressure, or heart rate.

### SUMMARY OF THE INVENTION

The present invention also relates to a method of measuring a physiological parameter of an individual, including collecting a plurality of sensor signals from at least one sensor in electronic communication with a sensor device worn on a body of the individual. The sensors is a physiological sensor which utilizes an output which is used to predict the state parameter of the individual. A method is disclosed that can help emergency care workers determine if a sick or wounded individual has reached a critical state. The method involves continuously collecting physiological data from an individual and relating this data to a critical care parameter, such as the existence of a traumatic injury or illness. In one embodiment, the collected data is analyzed with a mathematical operation to determine the presence of a critical state.

Also disclosed is a system that can help emergency care workers determine if a sick or wounded individual has reached a critical state. The system may be automated and is also adaptable or applicable to measuring a number of physiological parameters and reporting the same and derivations of such parameters. The preferred embodiment, a system to derive a critical care parameter, is directed to determining the acute health state of an individual. In other embodiments, the system may allow for early identification of illness and early corrective action.

In particular, the invention, according to one aspect, relates to an apparatus used in conjunction with a software platform for monitoring certain physiological measures. These measures are then transformed into values of the measure of a critical parameter, such as heart rate or oxygen debt, using mathematical techniques which then have predictive value in regards to outcome in response to injury and illness.

The management system utilizes an apparatus on the body that continuously monitors the certain physiological parameters, such as heat given off by a user's body in addition to motion, skin temperature and conductivity. Because the apparatus is continuously worn, data is collected during any physical activity performed by the user, including exercise activity and daily life activity. The apparatus is further designed for comfort and convenience so that long term wear is not unreasonable within a wearer's lifestyle activities. It is to be specifically noted that the apparatus is designed for both continuous and long term wear. In one aspect, the apparatus is utilized by an individual before the onset of trauma so that baseline data may be collected. In an additional embodiment, the data collected by the apparatus is uploaded to the software platform for determining the existence of a critical care state. The measured data may be collected by the processor within the sensor device, a cell phone or other second device that wirelessly communicates, such as RF, IR, Bluetooth, WiFi, Wimax, RFiD. The collection may occur utilizing the sensor device and either this second device or in collaboration between the two devices, i.e., shared processing. These devices then determine the state, level of the criticality of the patient, etc.

The system that is disclosed also provides an easy process for the entry and tracking of physical information. The user may choose from several methods of information input, such as direct, automatic, or manual input.

The combination of the information collected from the apparatus and the information entered by the user is used to provide feedback information regarding the user's physical state. Because of the accuracy of the information, the user or a third party can make immediate treatment decisions. The system can predict data indicative of human physiological parameters including energy expenditure and caloric intake for any given relevant time period as well as other detected and derived physiological or contextual information.

In an additional embodiment, an apparatus is disclosed for monitoring certain identified human status parameters which includes at least one sensor adapted to be worn on an individual's body. A preferred embodiment utilizes a combination of sensors to provide more accurately sensed data, with the output of the multiple sensors being utilized in the derivation of additional data. The sensor or sensors utilized by the apparatus may include a physiological sensor selected from the group consisting of respiration sensors, temperature sensors, heat flux sensors, body conductance sensors, body resistance sensors, body potential sensors, brain activity sensors, blood pressure sensors, body impedance sensors, body motion sensors, oxygen consumption sensors, body chemistry sensors, body position sensors, body pressure sensors, light absorption sensors, body sound sensors, piezoelectric sensors, electrochemical sensors, strain gauges, and optical sensors. The apparatus also includes a processor that receives at least a portion of the data indicative of the parameters. The processor is adapted to generate derived data from at least a portion of the data.

The apparatus may further include a housing adapted to be worn on the individual's body. The apparatus may further include a flexible body supporting the housing having first and second members that are adapted to wrap around a portion of the individual's body. The flexible body may support one or more of the sensors. The apparatus may further include wrapping means coupled to the housing for maintaining contact between the housing and the individual's body, and the wrapping means may support one or more of the sensors.

Another embodiment of the apparatus includes a central monitoring unit remote from the at least two sensors that includes a data storage device. The data storage device receives the derived data from the processor and retrievably stores the derived data therein. The apparatus also includes means for transmitting information based on the derived data from the central monitoring unit to a recipient, which recipient may include the individual or a third party authorized by the individual. The processor may be supported by a housing adapted to be worn on the individual's body, or alternatively may be part of the central monitoring unit.

In one embodiment of either the method, system or apparatus, the first function recognizes one or more contexts based on the first set of signals and one or more of the second functions is chosen based on the one or more recognized contexts. The outputs of the chosen second functions are used to predict the state parameter of the individual. In another embodiment, the first function recognizes each of a plurality of contexts based on the first set of signals and each of the one or more second functions corresponds to one of the contexts. The first function assigns a weight to each of the one or more second functions based on a recognition probability associated with the corresponding context, and the outputs of the one or more second functions and the weights are used to predict the state parameter of the individual. The outputs may be combined in a post processing step to predict the state parameter. In addition, in either the apparatus or the method, the state parameter may be caloric expenditure the second functions may be regression algorithms, the contexts may comprise rest and active and, the first function may comprise a naïve Bayesian classifier. Where the state parameter is caloric expenditure, caloric consumption data for the individual may be generated and information based on the caloric expenditure data and the caloric consumption data may be displayed, such as energy balance data, rate of weight loss or gain, or information relating to one or more goals of the individual.

In one embodiment of the apparatus, the processor and the memory are included in a wearable sensor device. In another embodiment, the apparatus includes a wearable sensor device, the processor and the memory being included in a computing device located separately from the sensor device, wherein the sensor signals are transmitted from the sensor device to the computing device.

The present invention also relates to a method of making software for an apparatus for measuring a state parameter of an individual including providing a first sensor device, the first sensor device receiving a plurality of signals from at least two sensors, using the first sensor device to create a first function and one or more second functions, each of the one or more second functions having an output, the first function utilizing a first set of signals based on one or more of the plurality of sensor signals to determine how a second set of signals based on one or more of the plurality of sensor signals is utilized in the one or more second functions, wherein one or more of the outputs are used to predict the state parameter of the individual. The method further includes creating the software including instructions for: (i) receiving a second plurality of signals collected by a second sensor device substantially structurally identical to the first sensor device for a period of time; (ii) utilizing a third set of signals based on one or more of the second plurality of sensor signals in the first function to determine how a fourth set of signals based on one or more of the second plurality of sensor signals is utilized in the one or more second functions; and (iii) utilizing the one or more outputs produced by the one or more second functions from the fourth set of signals to predict the state parameter of the individual. In the method, the step of using the sensor device to create the first function and the one or more second functions may include gathering a first set of the plurality of signals under conditions where the state parameter is present, contemporaneously gathering gold standard data relating to the state parameter, and using one or more machine learning techniques to generate the first function and the one or more second functions from the first set of the plurality of signals and the gold standard data. In addition, the first function may recognize one or more contexts based on the first set of signals and one or more of the second functions may be chosen based on the one or more recognized contexts, wherein the outputs of the chosen second functions are used to predict the state parameter of the individual. Alternatively, the first function may recognize each of a plurality of contexts based on the first set of signals and each of the one or more second functions may correspond to one of the contexts, wherein the first function assigns a weight to each of the one or more second functions based on a recognition probability associated with the corresponding context, and wherein the outputs of the one or more second functions and the weights are used to predict the state parameter of the individual.

One specific embodiment of the present invention relates to a method of measuring energy expenditure of an individual including collecting a plurality of sensor signals from at least one of a body motion sensor, a heat flux sensor, a skin conductance sensor, and a skin temperature sensor, each in electronic communication with a sensor device worn on a body of the individual, and utilizing a first set of signals based on one or more of the plurality of sensor signals in one or more functions to predict the energy expenditure of the individual. The utilizing step may include utilizing the first set of signals in a first function, the first function determining how a second set of signals based on one or more of the plurality of sensor signals is utilized in one or more second functions, each of the one or more second functions having an output, wherein one or more of the outputs are used to predict the energy expenditure of the individual. In addition, the collecting step may include collecting the plurality of sensor signals from a body motion sensor, a heat flux sensor, and a skin conductance sensor, the second set of signals comprising a heat flux high gain average variance (HFvar), a vector sum of transverse and longitudinal accelerometer SADs (VSAD), and a galvanic skin response low gain (GSR), wherein the second functions have the form of A*VSAD+B*HF+C*GSR+D*BMR+E, wherein A, B, C, D and E are constants and BMR is a basal metabolic rate for the individual.

The present invention also relates to an apparatus for measuring energy expenditure of an individual including a processor, at least two of a body motion sensor, a heat flux sensor, a skin conductance sensor, and a skin temperature sensor in electronic communication with the processor, and a memory storing software executable by the processor. The software includes instructions for collecting a plurality of sensor signals from the at least two of a body motion sensor, a heat flux sensor, a skin conductance sensor, and a skin temperature sensor, and utilizing a first set of signals based on one or more of the plurality of sensor signals in one or more functions to predict the energy expenditure of the individual. The utilizing instruction may include utilizing the first set of signals in a first function, the first function determining how a second set of signals based on one or more of the plurality of sensor signals is utilized in one or more second functions, each of the one or more second functions having an output, wherein one or more of the outputs are used to predict the energy expenditure of the individual. The collecting instruction may include collecting the plurality of sensor signals from a body motion sensor, a heat flux sensor, and a skin conductance sensor, the second set of signals comprising a heat flux high gain average variance (HFvar), a vector sum of transverse and longitudinal accelerometer SADs (VSAD), and a galvanic skin response low gain (GSR), wherein the second functions have the form of A*VSAD+B*HF+C*GSR+D*BMR+E, wherein A, B, C, D and E are constants and BMR is a basal metabolic rate for the individual.

The present invention also relates to a method of making software for an apparatus for measuring energy expenditure of an individual, including providing a first sensor device, the first sensor device receiving a plurality of signals from at least two of a body motion sensor, a heat flux sensor, a skin conductance sensor, and a skin temperature sensor, and using the first sensor device to create one or more functions that predict the energy expenditure of the individual using a first set of signals based on one or more of the plurality of sensor signals. The method further includes creating the software including instructions for: (i) receiving a second plurality of signals collected by a second sensor device substantially structurally identical to the first sensor device for a period of time, the second sensor device receiving the second plurality of signals from at least two of a body motion sensor, a heat flux sensor, a skin conductance sensor, and a skin temperature sensor; and (ii) utilizing a second set of signals based on one or more of the second plurality of sensor signals in the one or more functions to predict the energy expenditure of the individual. The step of using the sensor device to create the one or more functions may include gathering a first set of the plurality of signals under conditions where energy expenditure data for the individual is present, contemporaneously gathering gold standard data relating to the energy expenditure data for the individual, and using one or more machine learning techniques to generate the one or more functions from the first set of the plurality of signals and the gold standard data. In addition, the utilizing instruction may include utilizing the second set of signals in a first function, the first function determining how a third set of signals based on one or more of the second plurality of sensor signals is utilized in one or more second functions, each of the one or more second functions having an output; wherein one or more of the outputs are used to predict the energy expenditure of the individual.

In yet another embodiment, the present invention relates to an apparatus for automatically measuring a first state parameter of an individual, including a processor, one or more sensors for generating one or more signals over a period of time, the processor receiving the one or more signals, and a memory storing software executable by the processor. The software includes instructions for inputting one or more signal channels based on the one or more signals into a first function having a first output that predicts one or more second state parameters of the individual and either the first state parameter or an indicator of the first state parameter, wherein the first state parameter may be obtained from the indicator based on a first relationship between the first state parameter and the indicator, inputting the one or more signal channels into a second function having a second output that predicts the one or more second state parameters but not the first state parameter or the indicator of the first state parameter, and obtaining either the first state parameter or the indicator from the first and second outputs based on a second relationship between the first function and the second function, and, if the indicator is obtained, obtaining the first state parameter from the indicator based on the first relationship.

The present invention also relates to a method of automatically measuring a first state parameter of an individual, including collecting for a period of time one or more signals from one or more sensors in electronic communication with a sensor device worn on a body of the individual, inputting one or more signal channels based on the one or more signals into a first function having a first output that predicts one or more second state parameters of the individual and either the first state parameter or an indicator of the first state parameter, wherein the first state parameter may be obtained from the indicator based on a first relationship between the first state parameter and the indicator, inputting the one or more signal channels into a second function having a second output that predicts the one or more second state parameters but not the first state parameter or the indicator of the first state parameter, and obtaining either the first state parameter or the indicator from the first and second outputs based on a second relationship between the first function and the second function, and, if the indicator is obtained, obtaining the first state parameter from the indicator based on the first relationship. The device may be worn on the body at areas such as the arm, chest, left chest, and femoral location

In yet another embodiment, the present invention relates to a method of making software for an apparatus for automatically measuring a first state parameter of an individual. The method includes providing a first sensor device, the first sensor device receiving one or more signals from one or more sensors, using the first sensor device to create a first function having a first output that predicts one or more second state parameters of the individual and either the first state parameter or an indicator of the first state parameter, wherein the first state parameter may be obtained from the indicator based on a first relationship between the first state parameter and the indicator, the first function taking as inputs one or more signal channels based on the one or more signals, and using the first sensor device to create a second function having a second output that predicts the one or more second state parameters but not the first state parameter or the indicator of the first state parameter, the second function taking as inputs the one or more signal channels. The method further includes creating the software including instructions for: (i) receiving a second one or more signals collected by a second sensor device substantially structurally identical to the first sensor device for a period of time; (ii) inputting a second one or more signal channels based on the second one or more signals into the first function and the second function for generating the first output and the second output, respectively; and (iii) obtaining either the first state parameter or the indicator from the first and second outputs generated in the inputting step based on a second relationship between the first function and the second function, and, if the indicator is obtained, obtaining the first state parameter from the indicator based on the first relationship. The step of using the sensor device to create the first function may include gathering a first set of the one or more signals under conditions where the second state parameters and either the first state parameter or the indicator are present, contemporaneously gathering gold standard data relating to the second state parameters and either the first state parameter or the indicator, and using one or more machine learning techniques to generate the first function from the first set of one or more signals and the gold standard data, and the step of using the sensor device to create the second function may include gathering a second set of the one or more signals under conditions where neither the first state parameter nor the indicator are present, contemporaneously gathering second gold standard data relating to the second state parameters but not the first state parameter or the indicator, and using one or more machine learning techniques to generate the second function from the second set of one or more signals and the second gold standard data.

The disclosures of the following U.S. patents or U.S. patent applications are herein incorporated by reference in their entirety: U.S. application Ser. No. 11/928,302, U.S. Application Ser. No. 10/940,889, U.S. Provisional Application Ser. No. 60/502,764, U.S. Provisional Application Ser. No. 60/510,013, U.S. Provisional Application Ser. No. 60/555,280, U.S. patent application Ser. No. 10/940,214, U.S. application Ser. No. 10/638,588, filed Aug. 11, 2003, U.S. application Ser. No. 09/602,537, U.S. application Ser. No. 09/595,660, U.S. Provisional Application No. 60/502,764, U.S. Provisional Application No. 50/555,280, U.S. patent application Ser. No. 10/682,293, U.S. Provisional Application No. 60/417,163 and U.S. Provisional Application No. 61/116,364.

### BRIEF DESCRIPTION OF THE DRAWINGS

Further features and advantages of the present invention will be apparent upon consideration of the following detailed description of the present invention, taken in conjunction with the following drawings, in which like reference characters refer to like parts, and in which:

FIG. 1 is a diagram of an embodiment of a system for monitoring physiological data and lifestyle over an electronic network according to the present invention;

FIG. 2 is a block diagram of an embodiment of the sensor device shown in FIG. 1;

FIG. 3 is a block diagram of an embodiment of the central monitoring unit shown in FIG. 1;

FIG. 4 is a block diagram of an alternate embodiment of the central monitoring unit shown in FIG. 1;

FIG. 5 is a front view of a specific embodiment of the sensor device shown in FIG. 1;

FIG. 6 is a back view of a specific embodiment of the sensor device shown in FIG. 1;

FIG. 7 is a side view of a specific embodiment of the sensor device shown in FIG. 1;

FIG. 8 is a bottom view of a specific embodiment of the sensor device shown in FIG. 1;

FIGS. 9 and 10 are front perspective views of a specific embodiment of the sensor device shown in FIG. 1;

FIG. 11 is an exploded side perspective view of a specific embodiment of the sensor device shown in FIG. 1;

FIG. 12 is a side view of the sensor device shown in FIGS. 5 through 11 inserted into a battery recharger unit;

FIG. 13 is a block diagram illustrating all of the components either mounted on or coupled to the printed circuit board forming a part of the sensor device shown in FIGS. 5 through 11;

FIG. 14 is a block diagram showing the format of algorithms that are developed according to an aspect of the present invention;

FIG. 15 is a block diagram illustrating an example algorithm for predicting energy expenditure according to the present invention;

FIG. 16A is a front view of a specific embodiment of the sensor device;

FIG. 16B is an illustration of the device of 16A when worn on the arm of a subject;

FIGS. 17A and 17B are a comparison of metabolic cart EE and predicted EE in a level 1 trauma patient in a bedside situation;

FIGS. 18A and 18B are a comparison of shock index and predicted EE in a level 1 trauma bedside situation; and

FIGS. 19A, 19B and 19C are back, front and back views, respectively, of the left arm showing electrode placement locations according to an aspect of the present invention;

FIGS. 20A and 20B are back and front views, respectively, of the right arm showing electrode placement locations according to an aspect of the present invention;

FIGS. 20C, 20D and 20E are front, back and front views, respectively of the torso showing electrode placement locations according to an aspect of the present invention;

FIG. 21 is a block diagram of a circuit for detecting an ECG signal from according to an embodiment of the present invention;

FIGS. 22A and 22B are circuit diagrams of first and second embodiments of the bias/coupling network shown in FIGS. 21 and 24;

FIG. 22C is a circuit diagram of a first stage amplifier design;

FIG. 23 is a circuit diagram of one embodiment of the filter shown in FIGS. 4 and 7;

FIG. 24 is a block diagram of a circuit for detecting an ECG signal from according to an alternate embodiment of the present invention;

FIGS. 24A through 24D are diagrammatic representations of detected ECG signals through various stages of processing;

FIGS. 24E through 24H are diagrammatic representations of detected ECG signals through various stages of beat detection;

FIGS. 25A through 25F are block diagrams of alternative circuits for detecting an ECG signal from according to an alternate embodiment of the present invention;

FIG. 26 is a diagram of a typical peak forming a part of the signal generated according to the present invention;

FIGS. 26 and 27A and 27B are diagrams of a typical up-down-up sequence forming a part of the signal generated according to the present invention;

FIG. 28 is a graph illustrating measured ECG signal as a function of time

FIG. 29 is a bottom plan view of one embodiment of the armband body monitoring device;

FIG. 30 is a bottom plan view of a second embodiment of the armband body monitoring device;

FIG. 31 is a bottom plan view of a third embodiment of the armband body monitoring device;

FIG. 32 is a bottom plan view of a fourth embodiment of the armband body monitoring device;

FIG. 33 is a bottom plan view of a fifth embodiment of the armband body monitoring device;

FIG. 34 is a bottom plan view of a sixth embodiment of the armband body monitoring device;

FIG. 35 is a bottom plan view of a seventh embodiment of the armband body monitoring device;

FIG. 36 is an isometric view of the seventh embodiment of the armband body monitoring device mounted upon a human arm;

FIG. 37 is an isometric view of an eighth embodiment of the armband body monitoring device;

FIG. 38A is a top plan view of a ninth embodiment of the armband body monitoring device;

FIG. 38B is a bottom plan view of a ninth embodiment of the armband body monitoring device;

FIG. 38C is a sectional view of the embodiment of FIG. 38B taken along line A-A;

FIGS. 39A-39H are examples of sensor data averaged over LBNP/exercise severity; and

FIGS. 40A and 40B are graphical examples of armband sensors per each individual.

### DESCRIPTION OF THE PREFERRED EMBODIMENTS

In general, the device and method of the present invention utilizes development of mathematic formulas and/or algorithms to determine the presence of a critical care parameter. As used herein, a critical care parameter is one that indicates the existence of a critical illness or injury. Such illnesses or injury can include, but are not limited to, the following: 1) non-traumatic hemorrhage 2) traumatic hemorrhage; 3) acute and chronic heart failure including myocardial infarction and acute arhythmias; 4) cardiac arrest and cardiogenic shock; 5) severe bacterial, viral and fungal infection of the skin/soft tissue, brain, lung, abdominal organs, and bone; 6) sepsis, severe sepsis, septic shock; 7) wounds and burns; 8) metabolic derangements such as hyper and hypothryoid, adrenal insufficiency, diabetic ketoacidosis; 9) hyper and hypothermia; 10) preeclampsia and eclampsia; 11) seizures and status epilepticus; 12) drowning; 13) acute respiratory failure including asthma, emphysema, chronic obstructive pulmonary disease, airway obstructions; 14) pulmonary embolism; 15) traumatic brain injury; 16) spinal cord injury; 17) stroke or ischemic and hemorrhagic; 18) cerebral aneurysm; 20) limb ischemia; 21) coagulopathies; 22) acute neuromuscular disease/failure; 24) acute poisonings such as carbon monoxide, hydrogen sulfide, cyanide, cardiovascular medications, alcohols, antidepressants, etc.; 25) vasoocclusive crisis; and 26) tumor lysis syndrome.

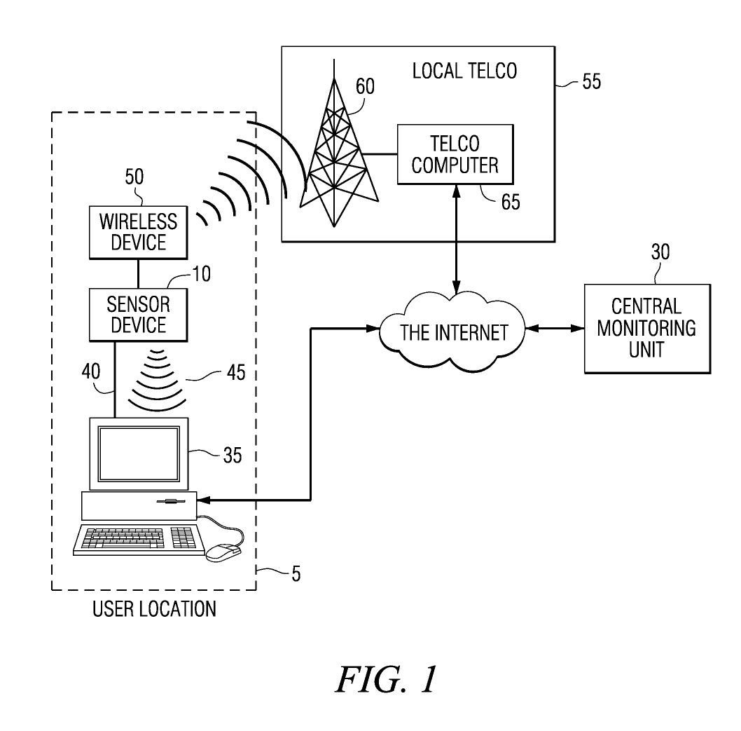

In one aspect of the present invention, data relating to the physiological state and certain contextual parameters of an individual are collected and transmitted, either subsequently or in real-time, to a site, preferably remote from the individual, where it is stored for later manipulation and presentation to a recipient, preferably over an electronic network such as the Internet. Referring to FIG. 1, located at user location 5 is sensor device 10 adapted to be placed in proximity with at least a portion of the human body. Sensor device 10 is preferably worn by an individual user on his or her body, for example as part of a garment such as a form fitting shirt, or as part of an arm band or the like. Sensor device 10, includes one or more sensors, which are adapted to generate signals in response to physiological characteristics of an individual, and a microprocessor. Proximity as used herein means that the sensors of sensor device 10 are separated from the individual's body by a material or the like, or a distance such that the capabilities of the sensors are not impeded. While in other embodiments, Sensor Device 10 is meant to comprise a device having all sensing, and optionally, processing capabilities therein, other embodiments allow for the sensing capabilities and processing capabilities to be spread across separate devices having partial or complete capabilities as those described herein for the Sensor Device 10 in electronic communication with one another.

Sensor device 10 generates data indicative of various physiological parameters of an individual, such as the individual's heart rate, pulse rate, beat-to-beat heart variability, EKG or ECG, body impedance, respiration rate, skin temperature, core body temperature, heat flow off the body, galvanic skin response or GSR, EMG, EEG, EOG, blood pressure, body fat, hydration level, activity level, oxygen consumption, glucose or blood sugar level, body position, pressure on muscles or bones, and UV radiation exposure and absorption. In certain cases, the data indicative of the various physiological parameters is the signal or signals themselves generated by the one or more sensors and in certain other cases the data is calculated by the microprocessor based on the signal or signals generated by the one or more sensors. Methods for generating data indicative of various physiological parameters and sensors to be used therefor are well known. Table 1 provides several examples of such well known methods and shows the parameter in question, an example method used, an example sensor device used, and the signal that is generated. Table 1 also provides an indication as to whether further processing based on the generated signal is required to generate the data.

TABLE 1

| | | | | Further |
|---|---|---|---|---|
| Parameter | Example Method | Example Sensor | Signal | Processing |
| Heart Rate | EKG | 2 Electrodes | DC Voltage | Yes |
| Pulse Rate | BVP | LED Emitter and | Change in Resistance | Yes |
| | | Optical Sensor | | |
| Beat-to-Beat | Heart Beats | 2 Electrodes | DC Voltage | Yes |
| Variability | | | | |
| EKG | Skin Surface | 3-10 Electrodes | DC Voltage | No* |
| | Potentials | | | (depending |
| | | | | on |
| | | | | location) |
| Respiration Rate | Chest Volume | Strain Gauge | Change in Resistance | Yes |
| | Change | | | |
| Skin Temperature | Surface | Thermistors | Change in Resistance | Yes |
| | Temperature | | | |
| | Probe | | | |
| Core Temperature | Esophageal or | Thermistors | Change in Resistance | Yes |
| | Rectal Probe | | | |
| Heat Flow | Heat Flux | Thermopile | DC Voltage | Yes |
| Galvanic Skin | Skin Conductance | 2 Electrodes | Conductance | No |
| Response | | | | |
| EMG | Skin Surface | 3 Electrodes | DC Voltage | No |
| | Potentials | | | |
| EEG | Skin Surface | Multiple Electrodes | DC Voltage | Yes |
| | Potentials | | | |
| EOG | Eye Movement | Thin Film | DC Voltage | Yes |
| | | Piezoelectric | | |
| | | Sensors | | |
| Blood Pressure | Non-Invasive | Electronic | Change in Resistance | Yes |
| | Korotkuff Sounds | Sphygromarometer | | |
| Body Fat | Body Impedance | 2 Active Electrodes | Change in Impedance | Yes |
| Activity in | Body Movement | Accelerometer | DC Voltage, | Yes |
| Interpreted G | | | Capacitance Changes | |
| Shocks per Minute | | | | |
| Activity | Body Movement | Accelerometer | DC Voltage, | Yes |
| | | | Capacitance Changes | |
| Oxygen | Oxygen Uptake | Electro-chemical | DC Voltage Change | Yes |
| Consumption | | | | |
| Glucose Level | Non-Invasive | Electro-chemical | DC Voltage Change | Yes |
| CO₂ Levels | Non-Invasive | Electro-chemical | DC Voltage Change | Yes |
| NADH Levels | Non-Invasive | Optical | DC Voltage Change | Yes |
| | | Spectroscopy or | | |
| | | Fluorescence | | |
| | | Spectroscopy | | |
| Optical | Non-Invasive | Spectroscopy | DC Voltage Change | Yes |
| Plethysmography | | | | |
| Piezo Motions | Non-Invasive | Thin Film | DC Voltage Change | Yes |
| | | Piezoelectric | | |
| | | Sensors | | |
| Muscle Pressure | N/A | Thin Film | DC Voltage Change | Yes |
| and/or Blood | | Piezoelectric | | |
| Across a Vessel or | | Sensors | | |
| Artery | | | | |
| Bioimpedence | Non-Invasive | 2 Active Electrodes | Change in Impedance | Yes |
| UV Radiation | N/A | UV Sensitive Photo | DC Voltage Change | Yes |
| Absorption | | Cells | | |

It is to be specifically noted that a number of other types and categories of sensors may be utilized alone or in conjunction with those given above, including but not limited to relative and global positioning sensors for determination of location of the user; torque & rotational acceleration for determination of orientation in space; blood chemistry sensors; interstitial fluid chemistry sensors; bio-impedance sensors; invasive lactate sensors, and several contextual sensors, such as: pollen, humidity, ozone, acoustic, body and ambient noise and sensors adapted to utilize the device in a biofingerprinting scheme.

The types of data listed in Table 1 are intended to be examples of the types of data that can be generated by sensor device 10. It is to be understood that other types of data relating to other parameters can be generated by sensor device 10 without departing from the scope of the present invention.

The microprocessor of sensor device 10 may be programmed to summarize and analyze the data. For example, the microprocessor can be programmed to calculate an average, minimum or maximum heart rate or respiration rate over a defined period of time, such as ten minutes. Sensor device 10 may be able to derive information relating to an individual's physiological state based on the data indicative of one or more physiological parameters. Yet, it should be understood that the microprocessor is programmed to do much more. For example, the microprocessor of sensor device 10 is programmed to derive such information using known methods based on the data indicative of one or more physiological parameters. Table 2 provides a non-exhaustive list of the type of information that can be derived, and indicates some of the types of data that can be used as inputs for the derivation. The methods and techniques disclosed herein and particularly in U.S. patent application Ser. No. 10/682,293 enable each of the parameters below (among others) to be derived any combination of inputs signals disclosed below or herein. Thus, it should be understood that any sensed parameter disclosed herein, i.e., input signal to a derivation, can be used alone or in combination with any other to derive the derived parameters listed herein.

TABLE 2

| Derived Information | Input Data Signals |
|---|---|
| Ovulation | Skin temperature, core temperature, oxygen consumption |
| Sleep onset/wake | Beat-to-beat variability, heart rate, pulse rate, respiration |
| | rate, skin temperature, core temperature, heat flow, galvanic |
| | skin response, EMG, EEG, EOG, blood pressure, oxygen |
| | consumption |
| Calories burned | Heart rate, pulse rate, respiration rate, heat flow, activity, |
| | oxygen consumption |
| Basal metabolic rate | Heart rate, pulse rate, respiration rate, heat flow, activity, |
| | oxygen consumption |
| Basal temperature | Skin temperature, core temperature |
| Activity level | Heart rate, pulse rate, respiration rate, heat flow, activity, |
| | oxygen consumption |
| Stress level | EKG, beat-to-beat variability, heart rate, pulse rate, |
| | respiration rate, skin temperature, heat flow, galvanic skin |
| | response, EMG, EEG, blood pressure, activity, oxygen |
| | consumption |
| Relaxation level | EKG, beat-to-beat variability, heart rate, pulse rate, |
| | respiration rate, skin temperature, heat flow, galvanic skin |
| | response, EMG, EEG, blood pressure, activity, oxygen |
| | consumption |
| Maximum oxygen consumption rate | EKG, heart rate, pulse rate, respiration rate, heat flow, blood |
| | pressure, activity, oxygen consumption |
| Rise time or the time it takes to rise from | Heart rate, pulse rate, heat flow, oxygen consumption |
| a resting rate to 85% of a target maximum | |
| Time in zone or the time heart rate was | Heart rate, pulse rate, heat flow, oxygen consumption |
| above 85% of a target maximum | |
| Recovery time or the time it takes heart | Heart rate, pulse rate, heat flow, oxygen consumption |
| rate to return to a resting rate after heart | |
| rate was above 85% of a target maximum | |

Additionally, sensor device 10 may also generate data indicative of various contextual parameters relating to the individual. Deriving a “context” (and any roots or derivations of the term used herein) means generating data about the circumstance, condition, environment, or setting of an individual. As a non limiting example, sensor device 10 can generate data indicative of the air quality, sound level/quality, light quality or ambient temperature near the individual, the global positioning of the individual, whether someone is driving in a car, lying down, running or standing up. Some contextual derivations can also be properly classified as activities and will be apparent to skilled artisan when such is the case. Sensor device 10 may include one or more sensors for generating signals in response to contextual characteristics relating to the environment surrounding the individual, the signals ultimately being used to generate the type of data described above. Such sensors are well known, as are methods for generating contextual parametric data such as air quality, sound level/quality, ambient temperature and global positioning.

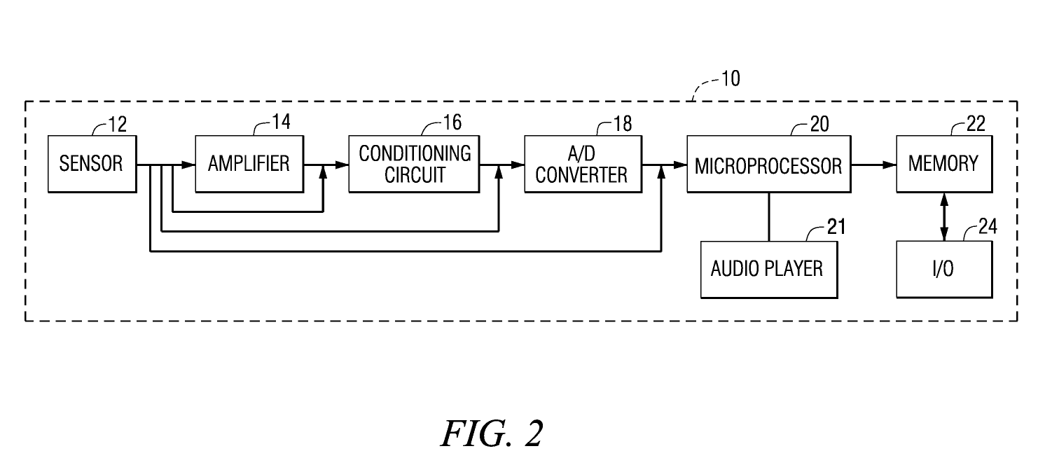

FIG. 2 is a block diagram of an embodiment of sensor device 10. Sensor device 10 includes at least one sensor 12 and microprocessor 20. Depending upon the nature of the signal generated by sensor 12, the signal can be sent through one or more of amplifier 14, conditioning circuit 16, and analog-to-digital converter 18, before being sent to microprocessor 20. For example, where sensor 12 generates an analog signal in need of amplification and filtering, that signal can be sent to amplifier 14, and then on to conditioning circuit 16, which may, for example, be a band pass filter. The amplified and conditioned analog signal can then be transferred to analog-to-digital converter 18, where it is converted to a digital signal. The digital signal is then sent to microprocessor 20. Alternatively, if sensor 12 generates a digital signal, that signal can be sent directly to microprocessor 20.

A digital signal or signals representing certain physiological and/or contextual characteristics of the individual user may be used by microprocessor 20 to calculate or generate data indicative of physiological and/or contextual parameters of the individual user. Microprocessor 20 is programmed to derive information relating to at least one aspect of the individual's physiological state. It should be understood that microprocessor 20 may also comprise other forms of processors or processing devices, such as a microcontroller, or any other device that can be programmed to perform the functionality described herein.

Optionally, central processing unit may provide operational control or, at a minimum, selection of an audio player device 21. As will be apparent to those skilled in the art, audio player 21 is of the type which either stores and plays or plays separately stored audio media. The device may control the output of audio player 21, as described in more detail below, or may merely furnish a user interface to permit control of audio player 21 by the wearer.

The data indicative of physiological and/or contextual parameters can, according to one embodiment of the present invention, be sent to memory 22, such as flash memory, where it is stored until uploaded in the manner to be described below. Although memory 22 is shown in FIG. 2 as a discrete element, it will be appreciated that it may also be part of microprocessor 20. Sensor device 10 also includes input/output circuitry 24, which is adapted to output and receive as input certain data signals in the manners to be described herein. Thus, memory 22 of the sensor device 10 will build up, over time, a store of data relating to the individual user's body and/or environment. That data is periodically uploaded from sensor device 10 and sent to remote central monitoring unit 30, as shown in FIG. 1, where it is stored in a database for subsequent processing and presentation to the user, preferably through a local or global electronic network such as the Internet. This uploading of data can be an automatic process that is initiated by sensor device 10 periodically or upon the happening of an event such as the detection by sensor device 10 of a heart rate below a certain level, or can be initiated by the individual user or some third party authorized by the user, preferably according to some periodic schedule, such as every day at 10:00 p.m. Alternatively, rather than storing data in memory 22, sensor device 10 may continuously upload data in real time.

The uploading of data from sensor device 10 to central monitoring unit 30 for storage can be accomplished in various ways. In one embodiment, the data collected by sensor device 10 is uploaded by first transferring the data to personal computer 35 shown in FIG. 1 by means of physical connection 40, which, for example, may be a serial connection such as an RS232 or USB port. This physical connection may also be accomplished by using a cradle, not shown, that is electronically coupled to personal computer 35 into which sensor device 10 can be inserted, as is common with many commercially available personal digital assistants. The uploading of data could be initiated by then pressing a button on the cradle or could be initiated automatically upon insertion of sensor device 10 or upon proximity to a wireless receiver. The data collected by sensor device 10 may be uploaded by first transferring the data to personal computer 35 by means of short-range wireless transmission, such as infrared or RF transmission, as indicated at 45.

Once the data is received by personal computer 35, it is optionally compressed and encrypted by any one of a variety of well known methods and then sent out over a local or global electronic network, preferably the Internet, to central monitoring unit 30. It should be noted that personal computer 35 can be replaced by any computing device that has access to and that can transmit and receive data through the electronic network, such as, for example, a personal digital assistant such as the Palm VII sold by Palm, Inc., or the Blackberry 2-way pager sold by Research in Motion, Inc.

Alternatively, the data collected by sensor device 10, after being encrypted and, optionally, compressed by microprocessor 20, may be transferred to wireless device 50, such as a 2-way pager or cellular phone, for subsequent long distance wireless transmission to local telco site 55 using a wireless protocol such as e-mail or as ASCII or binary data. Local telco site 55 includes tower 60 that receives the wireless transmission from wireless device 50 and computer 65 connected to tower 60. According to the preferred embodiment, computer 65 has access to the relevant electronic network, such as the Internet, and is used to transmit the data received in the form of the wireless transmission to the central monitoring unit 30 over the Internet. Although wireless device 50 is shown in FIG. 1 as a discrete device coupled to sensor device 10, it or a device having the same or similar functionality may be embedded as part of sensor device 10.

Sensor device 10 may be provided with a button to be used to time stamp events such as time to bed, wake time, and time of meals. These time stamps are stored in sensor device 10 and are uploaded to central monitoring unit 30 with the rest of the data as described above. The time stamps may include a digitally recorded voice message that, after being uploaded to central monitoring unit 30, are translated using voice recognition technology into text or some other information format that can be used by central monitoring unit 30. Note that in an alternate embodiment, these time-stamped events can be automatically detected.

In addition to using sensor device 10 to automatically collect physiological data relating to an individual user, a kiosk could be adapted to collect such data by, for example, weighing the individual, providing a sensing device similar to sensor device 10 on which an individual places his or her hand or another part of his or her body, or by scanning the individual's body using, for example, laser technology or an iStat blood analyzer. The kiosk would be provided with processing capability as described herein and access to the relevant electronic network, and would thus be adapted to send the collected data to the central monitoring unit 30 through the electronic network. A desktop sensing device, again similar to sensor device 10, on which an individual places his or her hand or another part of his or her body may also be provided. For example, such a desktop sensing device could be a lactate monitor in which an individual places his or her arm. An individual might also wear a ring having a sensor device 10 incorporated therein. A base, not shown, could then be provided which is adapted to be coupled to the ring. The desktop sensing device or the base just described may then be coupled to a computer such as personal computer 35 by means of a physical or short range wireless connection so that the collected data could be uploaded to central monitoring unit 30 over the relative electronic network in the manner described above. A mobile device such as, for example, a personal digital assistant, might also be provided with a sensor device 10 incorporated therein. Such a sensor device 10 would be adapted to collect data when mobile device is placed in proximity with the individual's body, such as by holding the device in the palm of one's hand, and upload the collected data to central monitoring unit 30 in any of the ways described herein.

An alternative embodiment includes the incorporation of third party devices, not necessary worn on the body, collect additional data pertaining to physiological conditions. Examples include portable blood analyzers, glucose monitors, weight scales, blood pressure cuffs, pulse oximeters, CPAP machines, portable oxygen machines, home thermostats, treadmills, cell phones and GPS locators. The system could collect from, or in the case of a treadmill or CPAP, control these devices, and collect data to be integrated into the streams for real time or future derivations of new parameters. An example of this is a pulse oximeter on the user's finger could help measure pulse and therefore serve a surrogate reading for blood pressure. Additionally, a user could utilize one of these other devices to establish baseline readings in order to calibrate the device.

Furthermore, in addition to collecting data by automatically sensing such data in the manners described above, individuals can also manually provide data relating to various parameters that is ultimately transferred to and stored at central monitoring unit 30. An individual user can access a web site maintained by central monitoring unit 30 and can directly input information relating to physiological conditions by entering text freely, by responding to questions posed by the web site, or by clicking through dialog boxes provided by the web site. Central monitoring unit 30 can also be adapted to periodically send electronic mail messages containing questions designed to elicit information relating to life activities to personal computer 35 or to some other device that can receive electronic mail, such as a personal digital assistant, a pager, or a cellular phone. The individual would then provide data relating to life activities to central monitoring unit 30 by responding to the appropriate electronic mail message with the relevant data. Central monitoring unit 30 may also be adapted to place a telephone call to an individual user in which certain questions would be posed to the individual user. The user could respond to the questions by entering information using a telephone keypad, or by voice, in which case conventional voice recognition technology would be used by central monitoring unit 30 to receive and process the response. The telephone call may also be initiated by the user, in which case the user could speak to a person directly or enter information using the keypad or by voice/voice recognition technology. Central monitoring unit 30 may also be given access to a source of information controlled by the user, for example the user's electronic calendar such as that provided with the Outlook product sold by Microsoft Corporation of Redmond, Wash., from which it could automatically collect information.

Feedback may also be provided to a user directly through sensor device 10 in a visual form, for example through an LED or LCD or by constructing sensor device 10, at least in part, of a thermochromatic plastic, in the form of an acoustic signal or in the form of tactile feedback such as vibration. Additionally, a reminder or alert can be issued in the event that a particular physiological parameter has been detected, such as high lactate levels have been encountered.

As will be apparent to those of skill in the art, it may be possible to download data from central monitoring unit 30 to sensor device 10. The flow of data in such a download process would be substantially the reverse of that described above with respect to the upload of data from sensor device 10. Thus, it is possible that the firmware of microprocessor 20 of sensor device 10 can be updated or altered remotely, i.e., the microprocessor can be reprogrammed, by downloading new firmware to sensor device 10 from central monitoring unit 30 for such parameters as timing and sample rates of sensor device 10. Also, the reminders/alerts provided by sensor device 10 may be set by the user using the web site maintained by central monitoring unit 30 and subsequently downloaded to the sensor device 10.

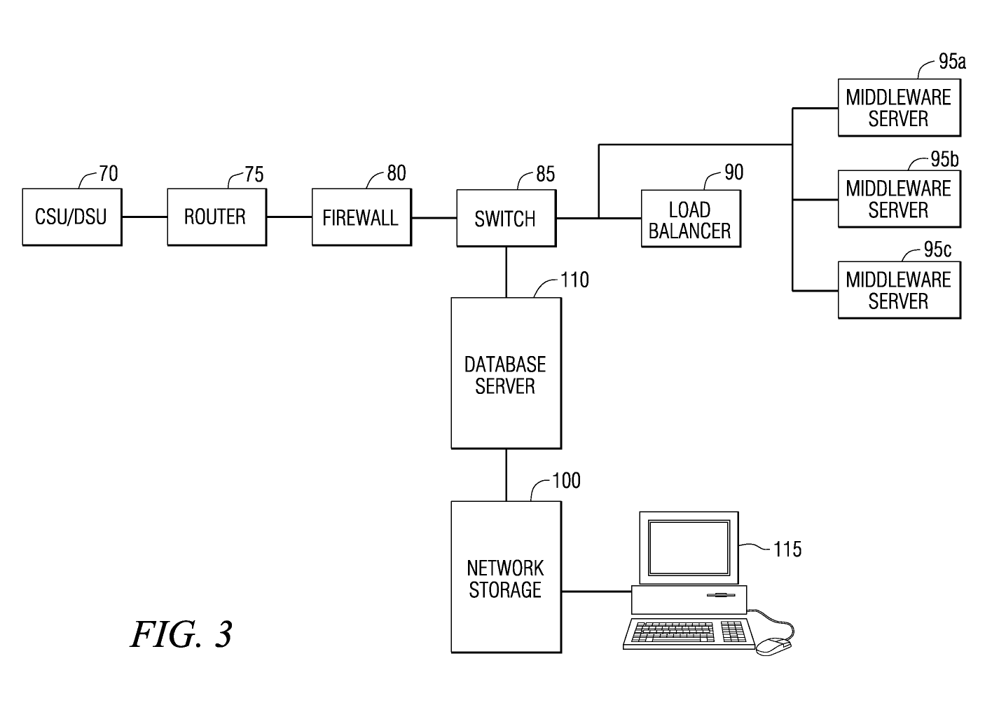

Referring to FIG. 3, a block diagram of an embodiment of central monitoring unit 30 is shown. Central monitoring unit 30 includes CSU/DSU 70 which is connected to router 75, the main function of which is to take data requests or traffic, both incoming and outgoing, and direct such requests and traffic for processing or viewing on the web site maintained by central monitoring unit 30. Connected to router 75 is firewall 80. The main purpose of firewall 80 is to protect the remainder of central monitoring unit 30 from unauthorized or malicious intrusions. Switch 85, connected to firewall 80, is used to direct data flow between middleware servers 95a through 95c and database server 110. Load balancer 90 is provided to spread the workload of incoming requests among the identically configured middleware servers 95a through 95c. Load balancer 90, a suitable example of which is the F5 Serverlron product sold by Foundry Networks, Inc. of San Jose, Calif., analyzes the availability of each middleware server 95a through 95c, and the amount of system resources being used in each middleware server 95a through 95c, in order to spread tasks among them appropriately.

Central monitoring unit 30 includes network storage device 100, such as a storage area network or SAN, which acts as the central repository for data. In particular, network storage device 100 comprises a database that stores all data gathered for each individual user in the manners described above. An example of a suitable network storage device 100 is the Symmetrix product sold by EMC Corporation of Hopkinton, Mass. Although only one network storage device 100 is shown in FIG. 3, it will be understood that multiple network storage devices of various capacities could be used depending on the data storage needs of central monitoring unit 30. Central monitoring unit 30 also includes database server 110 which is coupled to network storage device 100. Database server 110 is made up of two main components: a large scale multiprocessor server and an enterprise type software server component such as the 8/8i component sold by Oracle Corporation of Redwood City, Calif., or the 506 7 component sold by Microsoft Corporation of Redmond, Wash. The primary functions of database server 110 are that of providing access upon request to the data stored in network storage device 100, and populating network storage device 100 with new data. Coupled to network storage device 100 is controller 115, which typically comprises a desktop personal computer, for managing the data stored in network storage device 100.

Middleware servers 95a through 95c, a suitable example of which is the 22OR Dual Processor sold by Sun Microsystems, Inc. of Palo Alto, Calif., each contain software for generating and maintaining the corporate or home web page or pages of the web site maintained by central monitoring unit 30. As is known in the art, a web page refers to a block or blocks of data available on the World-Wide Web comprising a file or files written in Hypertext Markup Language or HTML, and a web site commonly refers to any computer on the Internet running a World-Wide Web server process. The corporate or home web page or pages are the opening or landing web page or pages that are accessible by all members of the general public that visit the site by using the appropriate uniform resource locator or URL. As is known in the art, URLs are the form of address used on the World-Wide Web and provide a standard way of specifying the location of an object, typically a web page, on the Internet. Middleware servers 95a through 95c also each contain software for generating and maintaining the web pages of the web site of central monitoring unit 30 that can only be accessed by individuals that register and become members of central monitoring unit 30. The member users will be those individuals who wish to have their data stored at central monitoring unit 30. Access by such member users is controlled using passwords for security purposes. Preferred embodiments of those web pages are described in detail below and are generated using collected data that is stored in the database of network storage device 100.

Middleware servers 95a through 95c also contain software for requesting data from and writing data to network storage device 100 through database server 110. When an individual user desires to initiate a session with the central monitoring unit 30 for the purpose of entering data into the database of network storage device 100, viewing his or her data stored in the database of network storage device 100, or both, the user visits the home web page of central monitoring unit 30 using a browser program such as Internet Explorer distributed by Microsoft Corporation of Redmond, Wash., and logs in as a registered user. Load balancer 90 assigns the user to one of the middleware servers 95a through 95c, identified as the chosen middleware server. A user will preferably be assigned to a chosen middleware server for each entire session. The chosen middleware server authenticates the user using any one of many well known methods, to ensure that only the true user is permitted to access the information in the database. A member user may also grant access to his or her data to a third party such as a health care provider or a personal trainer. Each authorized third party may be given a separate password and may view the member user's data using a conventional browser. It is therefore possible for both the user and the third party to be the recipient of the data.

When the user is authenticated, the chosen middleware server requests, through database server 110, the individual user's data from network storage device 100 for a predetermined time period. The predetermined time period is preferably thirty days. The requested data, once received from network storage device 100, is temporarily stored by the chosen middleware server in cache memory. The cached data is used by the chosen middleware server as the basis for presenting information, in the form of web pages, to the user again through the user's browser. Each middleware server 95a through 95c is provided with appropriate software for generating such web pages, including software for manipulating and performing calculations utilizing the data to put the data in appropriate format for presentation to the user. Once the user ends his or her session, the data is discarded from cache. When the user initiates a new session, the process for obtaining and caching data for that user as described above is repeated. This caching system thus ideally requires that only one call to the network storage device 100 be made per session, thereby reducing the traffic that database server 110 must handle. Should a request from a user during a particular session require data that is outside of a predetermined time period of cached data already retrieved, a separate call to network storage device 100 may be performed by the chosen middleware server. The predetermined time period should be chosen, however, such that such additional calls are minimized. Cached data may also be saved in cache memory so that it can be reused when a user starts a new session, thus eliminating the need to initiate a new call to network storage device 100.

As described in connection with Table 2, the microprocessor of sensor device 10 may be programmed to derive information relating to an individual's physiological state based on the data indicative of one or more physiological parameters. Central monitoring unit 30, and preferably middleware servers 95a through 95c, may also be similarly programmed to derive such information based on the data indicative of one or more physiological parameters.

It is also contemplated that a user will input additional data during a session, for example, information relating to the user's eating or sleeping habits. This additional data is preferably stored by the chosen middleware server in a cache during the duration of the user's session. When the user ends the session, this additional new data stored in a cache is transferred by the chosen middleware server to database server 110 for population in network storage device 100. Alternatively, in addition to being stored in a cache for potential use during a session, the input data may also be immediately transferred to database server 110 for population in network storage device 100, as part of a write-through cache system which is well known in the art.

Data collected by sensor device 10 shown in FIG. 1 is periodically uploaded to central monitoring unit 30. Either by long distance wireless transmission or through personal computer 35, a connection to central monitoring unit 30 is made through an electronic network, preferably the Internet. In particular, connection is made to load balancer 90 through CSU/DSU 70, router 75, firewall 80 and switch 85. Load balancer 90 then chooses one of the middleware servers 95a through 95c to handle the upload of data, hereafter called the chosen middleware server. The chosen middleware server authenticates the user using any one of many well known methods. If authentication is successful, the data is uploaded to the chosen middleware server as described above, and is ultimately transferred to database server 110 for population in the network storage device 100.

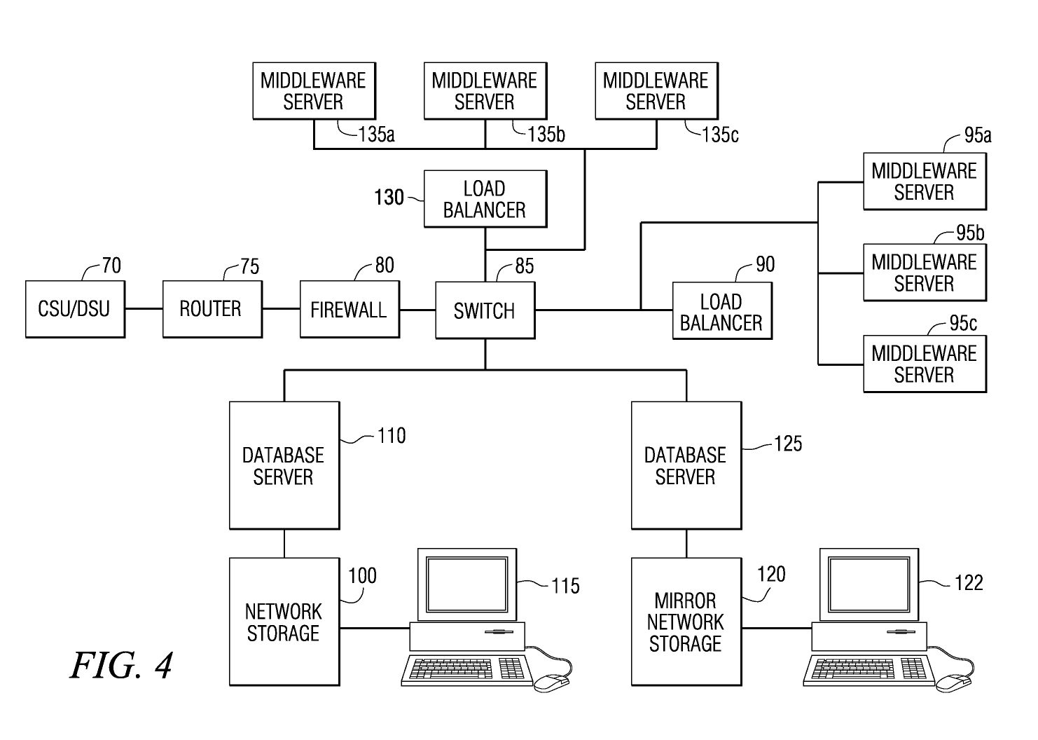

Referring to FIG. 4, an alternate embodiment of central monitoring unit 30 is shown. In addition to the elements shown and described with respect to FIG. 3, the embodiment of the central monitoring unit 30 shown in FIG. 4 includes a mirror network storage device 120 which is a redundant backup of network storage device 100. Coupled to mirror network storage device 120 is controller 122. Data from network storage device 100 is periodically copied to mirror network storage device 120 for data redundancy purposes.

Third parties such as insurance companies or research institutions may be given access, possibly for a fee, to certain of the information stored in mirror network storage device 120. Preferably, in order to maintain the confidentiality of the individual users who supply data to central monitoring unit 30, these third parties are not given access to such user's individual database records, but rather are only given access to the data stored in mirror network storage device 120 in aggregate form. Such third parties may be able to access the information stored in mirror network storage device 120 through the Internet using a conventional browser program. Requests from third parties may come in through CSU/DSU 70, router 75, firewall 80 and switch 85. In the embodiment shown in FIG. 4, a separate load balancer 130 is provided for spreading tasks relating to the accessing and presentation of data from mirror drive array 120 among identically configured middleware servers 135a through 135c. Middleware servers 135a through 135c each contain software for enabling the third parties to, using a browser, formulate queries for information from mirror network storage device 120 through separate database server 125. Middleware servers 135a through 135c also contain software for presenting the information obtained from mirror network storage device 120 to the third parties over the Internet in the form of web pages. In addition, the third parties can choose from a series of prepared reports that have information packaged along subject matter lines, such as various demographic categories.

As will be apparent to one of skill in the art, instead of giving these third parties access to the backup data stored in mirror network storage device 120, the third parties may be given access to the data stored in network storage device 100. Also, instead of providing load balancer 130 and middleware servers 135a through 135c, the same functionality, although at a sacrificed level of performance, could be provided by load balancer 90 and middleware servers 95a through 95c.

The Manager web pages comprise a utility through which central monitoring unit 30 provides various types and forms of data, commonly referred to as analytical status data, to the user that is generated from the data it collects or generates, namely one or more of: the data indicative of various physiological parameters generated by sensor device 10; the data derived from the data indicative of various physiological parameters; the data indicative of various contextual parameters generated by sensor device 10; and the data input by the user. Analytical status data is characterized by the application of certain utilities or algorithms to convert one or more of the data indicative of various physiological parameters generated by sensor device 10, the data derived from the data indicative of various physiological parameters, the data indicative of various contextual parameters generated by sensor device 10, and the data input by the user into calculated health, wellness and lifestyle indicators. As another example, skin temperature, heart rate, respiration rate, heat flow and/or GSR can be used to provide an indicator to the user of his or her stress level over a desired time period. As still another example, skin temperature, heat flow, beat-to-beat heart variability, heart rate, pulse rate, respiration rate, core temperature, galvanic skin response, EMG, EEG, EOG, blood pressure, oxygen consumption, ambient sound and body movement or motion as detected by a device such as an accelerometer can be used to provide indicators to the user of his or her sleep patterns over a desired time period.

In a variety of the embodiments described above, it is specifically contemplated that the data be input or detected by the system for derivation of the necessary data. One aspect of the present invention relates to a sophisticated algorithm development process for creating a wide range of algorithms for generating information relating to a variety of variables from the data received from the plurality of physiological and/or contextual sensors on sensor device 400. Such variables may include, without limitation, VO₂ levels, energy expenditure, including resting, active and total values, daily caloric intake, sleep states, including in bed, sleep onset, sleep interruptions, wake, and out of bed, and activity states, including exercising, sitting, traveling in a motor vehicle, and lying down, and the algorithms for generating values for such variables may be based on data from, for example, the 2-axis accelerometer, the heat flux sensor, the GSR sensor, the skin temperature sensor, the near-body ambient temperature sensor, and the heart rate sensor in the embodiment described above.

Note that there are several types of algorithms that can be computed. For example, and without limitation, these include algorithms for predicting user characteristics, continual measurements, durative contexts, instantaneous events, and cumulative conditions. User characteristics include permanent and semi-permanent parameters of the wearer, including aspects such as weight, height, and wearer identity. An example of a continual measurement is energy expenditure, which constantly measures, for example on a minute by minute basis, the number of calories of energy expended by the wearer. Durative contexts are behaviors that last some period of time, such as sleeping, driving a car, or jogging. Instantaneous events are those that occur at a fixed or over a very short time period, such as a heart attack or falling down. Cumulative conditions are those where the person's condition can be deduced from their behavior over some previous period of time. For example, if a person hasn't slept in 36 hours and hasn't eaten in 10 hours, it is likely that they are fatigued. Table 3 below shows numerous examples of specific personal characteristics, continual measurements, durative measurements, instantaneous events, and cumulative conditions.

TABLE 3

| | |
|---|---|
| personal characteristics | age, sex, weight, gender, athletic ability, |
| | conditioning, disease, height, susceptibility to |
| | disease, activity level, individual detection, |
| | handedness, metabolic rate, body composition |
| continual measurements | mood, beat-to-beat variability of heart beats, |
| | respiration, energy expenditure, blood glucose |
| | levels, level of ketosis, heart rate, stress levels, |
| | fatigue levels, alertness levels, blood pressure, |
| | readiness, strength, endurance, amenability to |
| | interaction, steps per time period, stillness |
| | level, body position and orientation, |
| | cleanliness, mood or affect, approachability, |
| | caloric intake, TEF, XEF, ‘in the zone’-ness, |
| | active energy expenditure, carbohydrate intake, |
| | fat intake, protein intake, hydration levels, |
| | truthfulness, sleep quality, sleep state, |
| | consciousness level, effects of medication, |
| | dosage prediction, water intake, alcohol |
| | intake, dizziness, pain, comfort, remaining |
| | processing power for new stimuli, proper use |
| | of the armband, interest in a topic, relative |
| | exertion, location, blood-alcohol level |
| durative measurements | exercise, sleep, lying down, sitting, standing, |
| | ambulation, running, walking, biking, |
| | stationary biking, road biking, lifting weights, |
| | aerobic exercise, anaerobic exercise, strength- |
| | building exercise, mind-centering activity, |
| | periods of intense emotion, relaxing, watching |
| | TV, sedentary, REM detector, eating, in-the- |
| | zone, interruptible, general activity detection, |
| | sleep stage, heat stress, heat stroke, amenable |
| | to teaching/learning, bipolar decompensation, |
| | abnormal events (in heart signal, in activity |
| | level, measured by the user, etc), startle level, |
| | highway driving or riding in a car, airplane |
| | travel, helicopter travel, boredom events, sport |
| | detection (football, baseball, soccer, etc), |
| | studying, reading, intoxication, effect of a drug |
| instantaneous events | falling, heart attack, seizure, sleep arousal |
| | events, PVCs, blood sugar abnormality, acute |
| | stress or disorientation, emergency, heart |
| | arrhythmia, shock, vomiting, rapid blood loss, |
| | taking medication, swallowing |
| cumulative conditions | Alzheimer's, weakness or increased likelihood |
| | of falling, drowsiness, fatigue, existence of |
| | ketosis, ovulation, pregnancy, disease, illness, |
| | fever, edema, anemia, having the flu, |
| | hypertension, mental disorders, acute |
| | dehydration, hypothermia, being-in-the-zone |

It will be appreciated that the present invention may be utilized in a method for doing automatic journaling of a wearer's physiological and contextual states. The system can automatically produce a journal of what activities the user was engaged in, what events occurred, how the user's physiological state changed over time, and when the user experienced or was likely to experience certain conditions. For example, the system can produce a record of when the user exercised, drove a car, slept, was in danger of heat stress, or ate, in addition to recording the user's hydration level, energy expenditure level, sleep levels, and alertness levels throughout a day.

According to the algorithm development process, linear or non-linear mathematical models or algorithms are constructed that map the data from the plurality of sensors to a desired variable. The process consists of several steps. First, data is collected by subjects wearing, for example, sensor device 400 who are put into situations as close to real world situations as possible, with respect to the parameters being measured, such that the subjects are not endangered and so that the variable that the proposed algorithm is to predict can, at the same time, be reliably measured using, for example, highly accurate medical grade lab equipment. This first step provides the following two sets of data that are then used as inputs to the algorithm development process: (i) the raw data from sensor device 400, and (ii) the data consisting of the verifiably accurate data measurements and extrapolated or derived data made with or calculated from the more accurate lab equipment, such as a VO₂ measurement device or indirect calorimeter. This verifiable data becomes a standard against which other analytical or measured data is compared. For cases in which the variable that the proposed algorithm is to predict relates to context detection, such as traveling in a motor vehicle, the verifiable standard data is provided by the subjects themselves, such as through information input manually into sensor device 400, a PC, or otherwise manually recorded. The collected data, i.e., both the raw data and the corresponding verifiable standard data, is then organized into a database and is split into training and test sets.

Next, using the data in the training set, a mathematical model is built that relates the raw data to the corresponding verifiable standard data. Specifically, a variety of machine learning techniques are used to generate two types of algorithms: 1) algorithms known as features, which are derived continuous parameters that vary in a manner that allows the prediction of the lab-measured parameter for some subset of the data points. The features are typically not conditionally independent of the lab-measured parameter e.g., VO₂ level information from a metabolic cart, douglas bag, or doubly labeled water, and 2) algorithms known as context detectors that predict various contexts, e.g., running, exercising, lying down, sleeping or driving, useful for the overall algorithm. A number of well known machine learning techniques may be used in this step, including artificial neural nets, decision trees, memory-based methods, boosting, attribute selection through cross-validation, and stochastic search methods such as simulated annealing and evolutionary computation.

After a suitable set of features and context detectors are found, several well known machine learning methods are used to combine the features and context detectors into an overall model. Techniques used in this phase include, but are not limited to, multilinear regression, locally weighted regression, decision trees, artificial neural networks, stochastic search methods, support vector machines, and model trees. These models are evaluated using cross-validation to avoid over-fitting.

At this stage, the models make predictions on, for example, a minute by minute basis. Inter-minute effects are next taken into account by creating an overall model that integrates the minute by minute predictions. A well known or custom windowing and threshold optimization tool may be used in this step to take advantage of the temporal continuity of the data. Finally, the model's performance can be evaluated on the test set, which has not yet been used in the creation of the algorithm. Performance of the model on the test set is thus a good estimate of the algorithm's expected performance on other unseen data. Finally, the algorithm may undergo live testing on new data for further validation.

Further examples of the types of non-linear functions and/or machine learning method that may be used in the present invention include the following: conditionals, case statements, logical processing, probabilistic or logical inference, neural network processing, kernel based methods, memory-based lookup including kNN and SOMs, decision lists, decision-tree prediction, support vector machine prediction, clustering, boosted methods, cascade-correlation, Boltzmann classifiers, regression trees, case-based reasoning, Gaussians, Bayes nets, dynamic Bayesian networks, HMMs, Kalman filters, Gaussian processes and algorithmic predictors, e.g. learned by evolutionary computation or other program synthesis tools.

Although one can view an algorithm as taking raw sensor values or signals as input, performing computation, and then producing a desired output, it is useful in one preferred embodiment to view the algorithm as a series of derivations that are applied to the raw sensor values. Each derivation produces a signal referred to as a derived channel. The raw sensor values or signals are also referred to as channels, specifically raw channels rather than derived channels. These derivations, also referred to as functions, can be simple or complex but are applied in a predetermined order on the raw values and, possibly, on already existing derived channels. The first derivation must, of course, only take as input raw sensor signals and other available baseline information such as manually entered data and demographic information about the subject, but subsequent derivations can take as input previously derived channels. Note that one can easily determine, from the order of application of derivations, the particular channels utilized to derive a given derived channel. Also note that inputs that a user provides on an Input/Output, or I/O, device or in some fashion can also be included as raw signals which can be used by the algorithms. In one embodiment, the raw signals are first summarized into channels that are sufficient for later derivations and can be efficiently stored. These channels include derivations such as summation, summation of differences, and averages. Note that although summarizing the high-rate data into compressed channels is useful both for compression and for storing useful features, it may be useful to store some or all segments of high rate data as well, depending on the exact details of the application. In one embodiment, these summary channels are then calibrated to take minor measurable differences in manufacturing into account and to result in values in the appropriate scale and in the correct units. For example, if, during the manufacturing process, a particular temperature sensor was determined to have a slight offset, this offset can be applied, resulting in a derived channel expressing temperature in degrees Celsius.

For purposes of this description, a derivation or function is linear if it is expressed as a weighted combination of its inputs together with some offset. For example, if G and H are two raw or derived channels, then all derivations of the form A*G+B*H+C, where A, B, and C are constants, is a linear derivation. A derivation is non-linear with respect to its inputs if it can not be expressed as a weighted sum of the inputs with a constant offset. An example of a nonlinear derivation is as follows: if G>7 then return H*9, else return H*3.5+912. A channel is linearly derived if all derivations involved in computing it are linear, and a channel is nonlinearly derived if any of the derivations used in creating it are nonlinear. A channel nonlinearly mediates a derivation if changes in the value of the channel change the computation performed in the derivation, keeping all other inputs to the derivation constant.

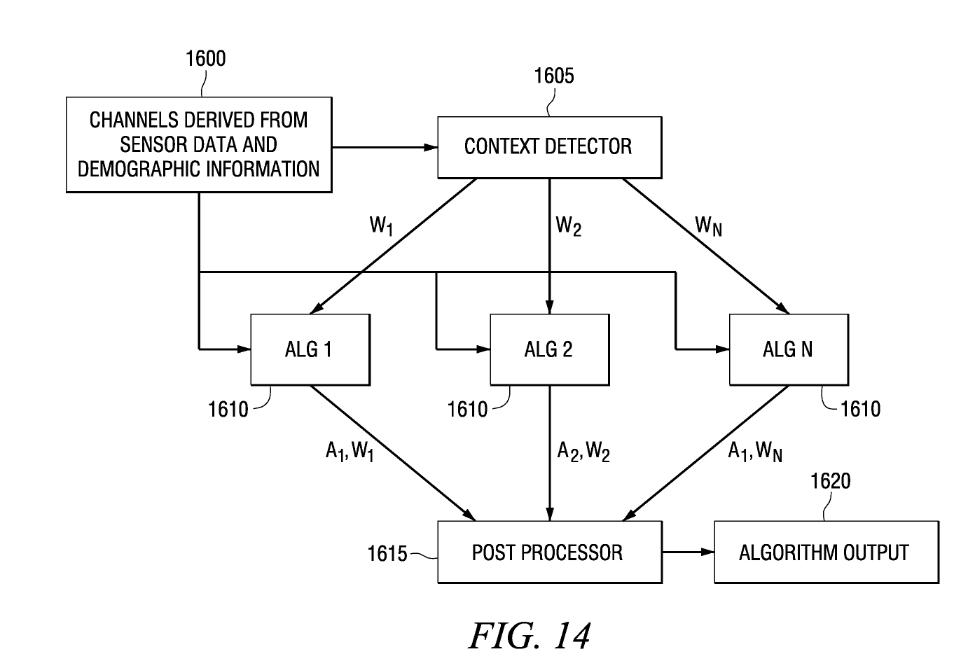

According to a preferred embodiment of the present invention, the algorithms that are developed using this process will have the format shown conceptually in FIG. 14. Specifically, the algorithm will take as inputs the channels derived from the sensor data collected by the sensor device from the various sensors, and demographic information for the individual as shown in box 1600. The algorithm includes at least one context detector 1605 that produces a weight, shown as W1 through WN, expressing the probability that a given portion of collected data, such as is collected over a minute, was collected while the wearer was in each of several possible contexts. Such contexts may include whether the individual was at rest or active. In addition, for each context, a regression algorithm 1610 is provided where a continuous prediction is computed taking raw or derived channels as input. The individual regressions can be any of a variety of regression equations or methods, including, for example, multivariate linear or polynomial regression, memory based methods, support vector machine regression, neural networks, Gaussian processes, arbitrary procedural functions and the like. Each regression is an estimate of the output of the parameter of interest in the algorithm, for example, energy expenditure. Finally, the outputs of each regression algorithm 1610 for each context, shown as A1 through AN, and the weights W1 through WN are combined in a post-processor 1615 which outputs the parameter of interest being measured or predicted by the algorithm, shown in box 1620. In general, the post-processor 1615 can consist of any of many methods for combining the separate contextual predictions, including committee methods, boosting, voting methods, consistency checking, or context based recombination.

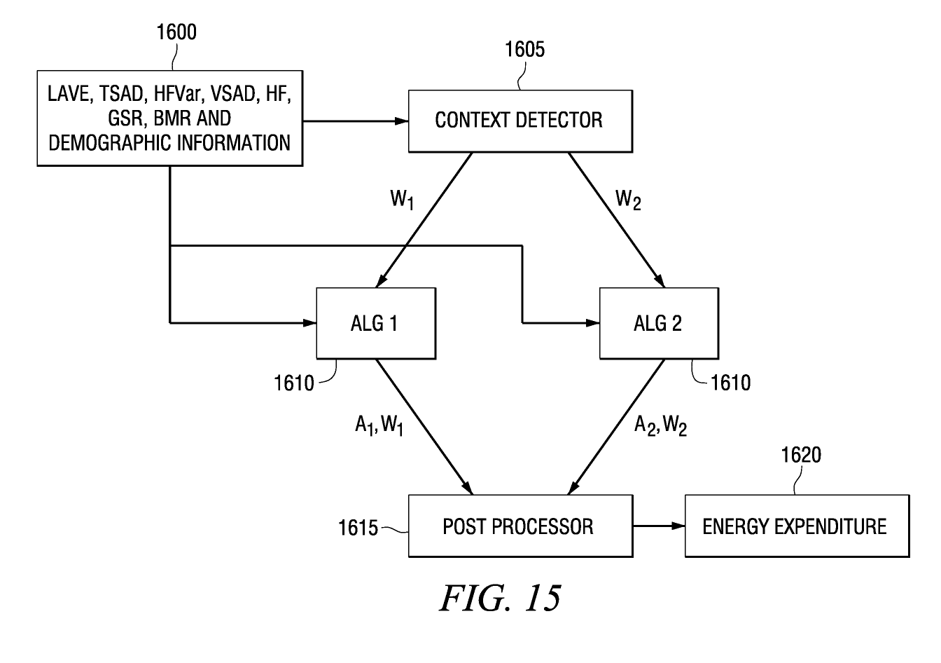

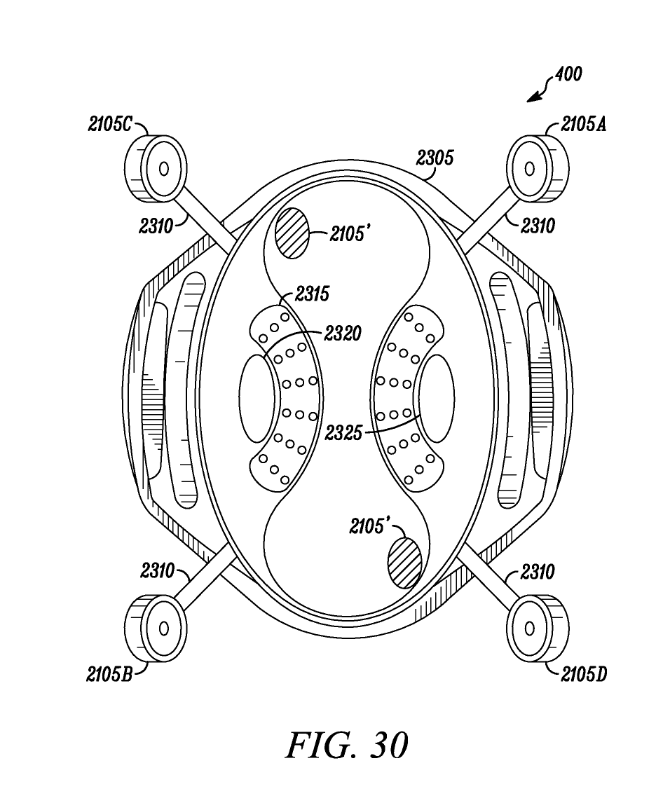

Referring to FIG. 15, an example algorithm for measuring the energy expenditure of an individual is shown. This example algorithm may be run on sensor device 400 having at least an accelerometer, a heat flux sensor and a GSR sensor, or an I/O device 1200 that receives data from such a sensor device as is disclosed in co-pending U.S. patent application Ser. No. 10/682,759, issued as U.S. Pat. No. 7,285,090, the specification of which is incorporated herein by reference. In this example algorithm, the raw data from the sensors is calibrated and numerous values based thereon, i.e., derived channels, are created. In particular, the following derived channels, shown at 1600 in FIG. 30, are computed from the raw signals and the demographic information: (1) longitudinal accelerometer average, or LAVE, based on the accelerometer data; (2) transverse accelerometer sum of average differences, or TSAD, based on the accelerometer data; (3) heat flux high gain average variance, or HFvar, based on heat flux sensor data; (4) vector sum of transverse and longitudinal accelerometer sum of absolute differences or SADs, identified as VSAD, based on the accelerometer data; (5) galvanic skin response, or GSR, in both low and combined gain embodiments; and (6) Basal Metabolic Rate or BMR. Context detector 1605 consists of a naïve Bayesian classifier that predicts whether the wearer is active or resting using the LAVE, TSAD, and HFvar derived channels. The output is a probabilistic weight, W1 and W2 for the two contexts rest and active. For the rest context, the regression algorithm 1610 is a linear regression combining channels derived from the accelerometer, the heat flux sensor, the user's demographic data, and the galvanic skin response sensor. The equation, obtained through the algorithm design process, is A*VSAD+B*HFvar+C*GSR+D*BMR+E, where A, B, C, D and E are constants. The regression algorithm 1610 for the active context is the same, except that the constants are different. The post-processor 1615 for this example is to add together the weighted results of each contextual regression. If A1 is the result of the rest regression and A2 is the result of the active regression, then the combination is just W1*A1+W2*A2, which is energy expenditure shown at 1620. In another example, a derived channel that calculates whether the wearer is motoring, that is, driving in a car at the time period in question might also be input into the post-processor 1615. The process by which this derived motoring channel is computed is algorithm 3. The post-processor 1615 in this case might then enforce a constraint that when the wearer is predicted to be driving by algorithm 3, the energy expenditure is limited for that time period to a value equal to some factor, e.g. 1.3 times their minute by minute basal metabolic rate.

This algorithm development process may also be used to create algorithms to enable the sensor device 400 to detect and measure various other parameters, including, without limitation, the following: (i) when an individual is suffering from duress, including states of unconsciousness, fatigue, shock, drowsiness, heat stress and dehydration; and (ii) an individual's state of readiness, health and/or metabolic status, such as in a military environment, including states of dehydration, under-nourishment and lack of sleep. In addition, algorithms may be developed for other purposes, such as filtering, signal clean-up and noise cancellation for signals measured by a sensor device as described herein. As will be appreciated, the actual algorithm or function that is developed using this method will be highly dependent on the specifics of the sensor device used, such as the specific sensors and placement thereof and the overall structure and geometry of the sensor device. Thus, an algorithm developed with one sensor device will not work as well, if at all, on sensor devices that are not substantially structurally identical to the sensor device used to create the algorithm.

Another aspect of the present invention relates to the ability of the developed algorithms to handle various kinds of uncertainty. Data uncertainty refers to sensor noise and possible sensor failures. Data uncertainty is when one cannot fully trust the data. Under such conditions, for example, if a sensor, for example an accelerometer, fails, the system might conclude that the wearer is sleeping or resting or that no motion is taking place. Under such conditions it is very hard to conclude if the data is bad or if the model that is predicting and making the conclusion is wrong. When an application involves both model and data uncertainties, it is very important to identify the relative magnitudes of the uncertainties associated with data and the model. An intelligent system would notice that the sensor seems to be producing erroneous data and would either switch to alternate algorithms or would, in some cases, be able to fill the gaps intelligently before making any predictions. When neither of these recovery techniques are possible, as was mentioned before, returning a clear statement that an accurate value can not be returned is often much preferable to returning information from an algorithm that has been determined to be likely to be wrong. Determining when sensors have failed and when data channels are no longer reliable is a non-trivial task because a failed sensor can sometimes result in readings that may seem consistent with some of the other sensors and the data can also fall within the normal operating range of the sensor.

Clinical uncertainty refers to the fact that different sensors might indicate seemingly contradictory conclusions. Clinical uncertainty is when one cannot be sure of the conclusion that is drawn from the data. For example, the accelerometers might indicate that the wearer is motionless, leading toward a conclusion of a resting user, the galvanic skin response sensor might provide a very high response, leading toward a conclusion of an active user, the heat flow sensor might indicate that the wearer is still dispersing substantial heat, leading toward a conclusion of an active user, and the heart rate sensor might indicate that the wearer has an elevated heart rate, leading toward a conclusion of an active user. An inferior system might simply try to vote among the sensors or use similarly unfounded methods to integrate the various readings. The present invention weights the important joint probabilities and determines the appropriate most likely conclusion, which might be, for this example, that the wearer is currently performing or has recently performed a low motion activity such as stationary biking

According to a further aspect of the present invention, a sensor device such as sensor device 400 may be used to automatically measure, record, store and/or report a parameter Y relating to the state of a person, preferably a state of the person that cannot be directly measured by the sensors. State parameter Y may be, for example and without limitation, calories consumed, energy expenditure, sleep states, hydration levels, ketosis levels, shock, insulin levels, physical exhaustion and heat exhaustion, among others. The sensor device is able to observe a vector of raw signals consisting of the outputs of certain of the one or more sensors, which may include all of such sensors or a subset of such sensors. As described above, certain signals, referred to as channels same potential terminology problem here as well, may be derived from the vector of raw sensor signals as well. A vector X of certain of these raw and/or derived channels, referred to herein as the raw and derived channels X, will change in some systematic way depending on or sensitive to the state, event and/or level of either the state parameter Y that is of interest or some indicator of Y, referred to as U, wherein there is a relationship between Y and U such that Y can be obtained from U. According to the present invention, a first algorithm or function f1 is created using the sensor device that takes as inputs the raw and derived channels X and gives an output that predicts and is conditionally dependent, expressed with the symbol [symbol] , on (i) either the state parameter Y or the indicator U, and (ii) some other state parameter(s) Z of the individual. This algorithm or function f1 may be expressed as follows:

f1(X) [symbol] U+Z

or

f1(X) [symbol] Y+Z

According to the preferred embodiment, f1 is developed using the algorithm development process described elsewhere herein which uses data, specifically the raw and derived channels X, derived from the signals collected by the sensor device, the verifiable standard data relating to U or Y and Z contemporaneously measured using a method taken to be the correct answer, for example highly accurate medical grade lab equipment, and various machine learning techniques to generate the algorithms from the collected data. The algorithm or function f1 is created under conditions where the indicator U or state parameter Y, whichever the case may be, is present. As will be appreciated, the actual algorithm or function that is developed using this method will be highly dependent on the specifics of the sensor device used, such as the specific sensors and placement thereof and the overall structure and geometry of the sensor device. Thus, an algorithm developed with one sensor device will not work as well, if at all, on sensor devices that are not substantially structurally identical to the sensor device used to create the algorithm or at least can be translated from device to device or sensor to sensor with known conversion parameters.

Next, a second algorithm or function f2 is created using the sensor device that takes as inputs the raw and derived channels X and gives an output that predicts and is conditionally dependent on everything output by f1 except either Y or U, whichever the case may be, and is conditionally independent, indicated by the symbol [symbol] , of either Y or U, whichever the case may be. The idea is that certain of the raw and derived channels X from the one or more sensors make it possible to explain away or filter out changes in the raw and derived channels X coming from non-Y or non-U related events. This algorithm or function f2 may be expressed as follows:

f2(X) [symbol] Z and (f2(X) [symbol] Y or f2(X) [symbol] U

Preferably, f2, like f1, is developed using the algorithm development process referenced above. f2, however, is developed and validated under conditions where U or Y, whichever the case may, is not present. Thus, the gold standard data used to create f2 is data relating to Z only measured using highly accurate medical grade lab equipment.

Thus, according to this aspect of the invention, two functions will have been created, one of which, f1, is sensitive to U or Y, the other of which, f2, is insensitive to U or Y. As will be appreciated, there is a relationship between f1 and f2 that will yield either U or Y, whichever the case may be. In other words, there is a function f3 such that f3 (f1, f2)=U or f3 (f1, f2)=Y. For example, U or Y may be obtained by subtracting the data produced by the two functions (U=f1−f2 or Y=f1−f2). In the case where U, rather than Y, is determined from the relationship between f1 and f2, the next step involves obtaining Y from U based on the relationship between Y and U. For example, Y may be some fixed percentage of U such that Y can be obtained by dividing U by some factor.

One skilled in the art will appreciate that in the present invention, more than two such functions, e.g. (f1, f2, f3, . . . f_n−1) could be combined by a last function f_n in the manner described above. In general, this aspect of the invention requires that a set of functions is combined whose outputs vary from one another in a way that is indicative of the parameter of interest. It will also be appreciated that conditional dependence or independence as used here will be defined to be approximate rather than precise.

It is known that total body metabolism is measured as total energy expenditure (TEE) according to the following equation:

TEE=BMR+AE+TEF+AT,

wherein BMR is basal metabolic rate, which is the energy expended by the body during rest such as sleep, AE is activity energy expenditure, which is the energy expended during physical activity, TEF is thermic effect of food, which is the energy expended while digesting and processing the food that is eaten, and AT is adaptive thermogenesis, which is a mechanism by which the body modifies its metabolism to extreme temperatures. It is estimated that it costs humans about 10% of the value of food that is eaten to process the food. TEF is therefore estimated to be 10% of the total calories consumed. Thus, a reliable and practical method of measuring TEF would enable caloric consumption to be measured without the need to manually track or record food related information. Specifically, once TEF is measured, caloric consumption can be accurately estimated by dividing TEF by 0.1 (TEF=0.1*Calories Consumed; Calories Consumed=TEF/0.1).

Preferably, the sensor device is in communication with a body motion sensor such as an accelerometer adapted to generate data indicative of motion, a skin conductance sensor such as a GSR sensor adapted to generate data indicative of the resistance of the individual's skin to electrical current, a heat flux sensor adapted to generate data indicative of heat flow off the body, a body potential sensor such as an ECG sensor adapted to generate data indicative of the rate or other characteristics of the heart beats of the individual, a free-living metabolite sensor adapted to measure metabolite levels such as glucose and/or lactate, and a temperature sensor adapted to generate data indicative of a temperature of the individual's skin. In this preferred embodiment, these signals, in addition the demographic information about the wearer, make up the vector of signals from which the raw and derived channels X are derived. Most preferably, this vector of signals includes data indicative of motion, resistance of the individual's skin to electrical current and heat flow off the body.

In one aspect, the present invention relates to a method and apparatus for measuring heart related parameters. A critical care parameter, such as those described above, may be derived from this measured information.

Conventional thinking in the field of cardiology/ECG is that an ECG signal must be measured across the heart, meaning with electrodes placed in two different quadrants of the heart's conventionally defined sagittal and transverse planes. A device and methodology are disclosed herein which permits the measurement of an ECG signal from certain pairs of points located within regions or areas of the human body previously considered inappropriate for such measurement. The device and methodology disclosed herein focus on the identification of certain locations on the body within the previously defined equivalence regions utilized for electrode location. Many of these electrode locations are within a single quadrant, i.e., when the electrode locations are connected geometrically directly through tissue, the line described thereby does not cross into another quadrant. In other words, certain points within one quadrant are correlated with the electropotential of the ECG signal conventionally associated with a different quadrant because the potential from the opposite side has been transported to that point internally through what appear to be low impedance non-homogeneous electropotential or electrical pathways through the body, which may be analogized as internal signal leads within the tissue. This methodology therefore focuses on two different aspects of the ECG signal, rather than more narrowly defining these aspects as emanating from certain quadrants of the body. Thus, contrary to the teachings of the prior art, an ECG signal may be detected and measured using pairs of electrodes placed within a single quadrant, but detecting a significant electrical potential difference between the two points. In other words, the two points are inequipotential with respect to one another. In most instances, it is more helpful to envision the electrode locations being located within independent regions of skin surface, separated by a boundary which may be planar or irregular.

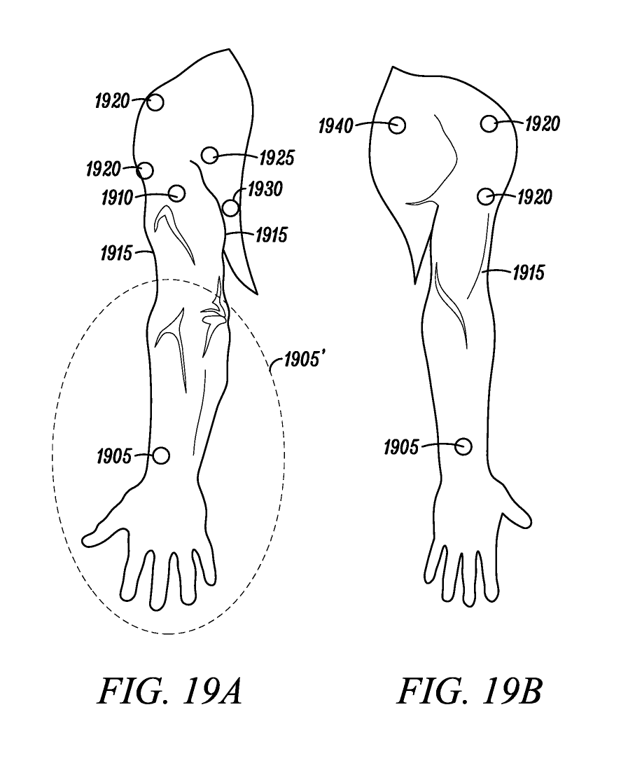

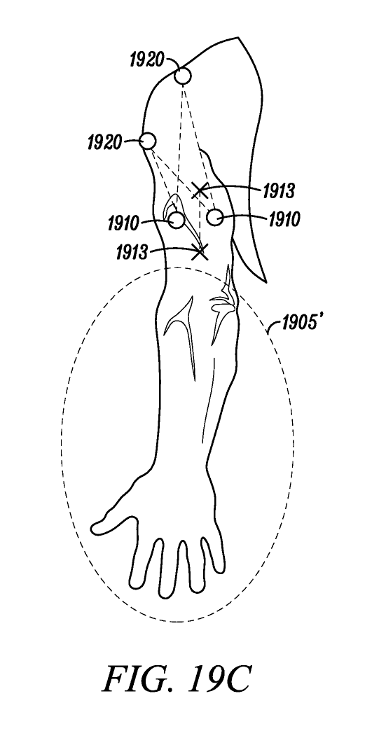

In the preferred embodiment of the present invention, pairs of locations on or near the left arm have been identified for placement of electrodes to detect the different aspects of the ECG signal. It is to be noted that similar sites within equivalence regions are found at a myriad of locations on the human body, including the right and left arms, the axillary area under the arms, the anterior femoral area adjacent the pelvis, the back of the base of the neck and the base of the spine. More specifically, certain locations on the left arm carry an aspect of the ECG signal and certain locations on or near the left arm carry a different aspect of the ECG signal. It is also to be specifically noted that anatomical names, especially names of muscles or muscle groups, are used to identify or reference locations on the body, though placement of the electrodes need only be applied to the skin surface directly adjacent these locational references and are not intended to be invasive. Referring now to FIGS. 19A and 19B, which are drawings of the back and front of the left arm, respectively, the inventors have found that the left wrist 1905, left triceps muscle 19110, and the left brachialis muscle 1915 are locations that, when paired with locations surrounding the deltoid muscle 1920, the teres major muscle 1925 and the latissimus dorsi muscle 1930, can produce an electrical potential signal that is related to the conventional signal measured between two quadrants. More specifically, the signal from these pairs of points on the left arm correlates with the QRS complex associated with the contraction of the ventricles.

Thus, by placing one electrode on the wrist 195, triceps muscle 1910 or the brachialis muscle 1915 and a second electrode on the deltoid muscle 1920, the teres major muscle 1925 or the latissimus dorsi muscle 1930, it is possible to detect the action potential of the heart and thus an ECG signal. The electrodes are preferably located near the central point of the deltoid and tricep muscles, are spaced approximately 130 mm and more particularly 70-80 mm apart and tilted at approximately 30-45 degrees toward the posterior of the arm from the medial line, with 30 degrees being most preferred. While certain specific preferred locations on or near the left arm have been described herein as being related to the electropotential of the second aspect of the ECG signal, it should be appreciated those locations are merely exemplary and that other locations on or near the left arm that are related to the electropotential of the second aspect of the ECG signal may also be identified by making potential measurements. It is further to be specifically noted that the entire lower arm section 5′ is identified as providing the same signal as wrist 1905. Referring now to FIG. 19C, four specific pairs of operative locations are illustrated, having two locations on the deltoid 20 and two locations on the various aspects of the tricep 1910. In one embodiment, the placement location is the juncture of the bicep and deltoid meet. The second electrode may then be placed anywhere on the deltoid. It is to be noted that the dashed lines between the locations indicate the operative pairings and that the solid and white dots represent the relative aspects of the ECG signal obtainable at those locations. Four possible combinations are shown which provide two aspects of the ECG signal. An inoperative pair, 1913 is illustrated to indicate that merely selecting particular muscles or muscle groups is not sufficient to obtain an appropriate signal, but that careful selection of particular locations is required.

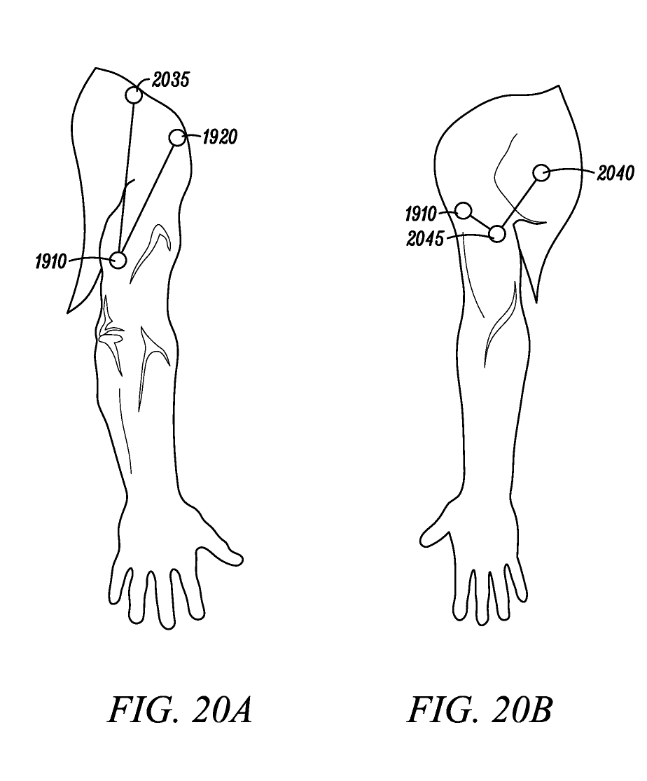

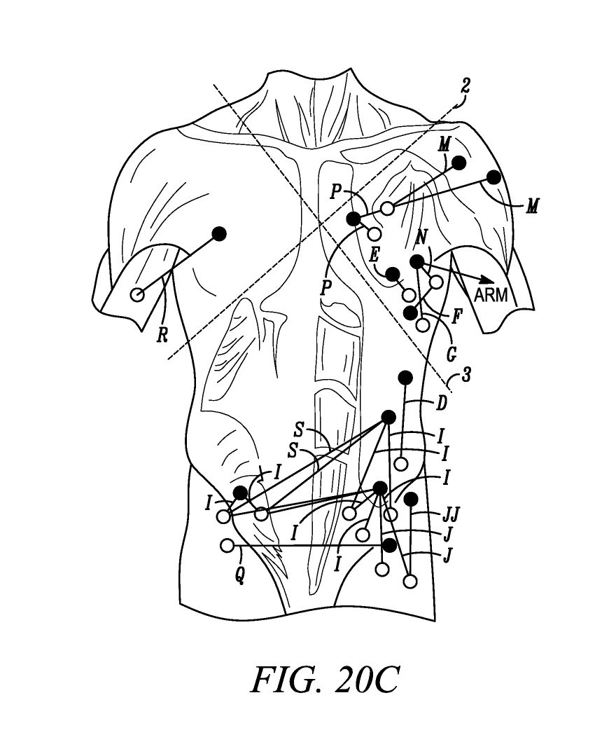

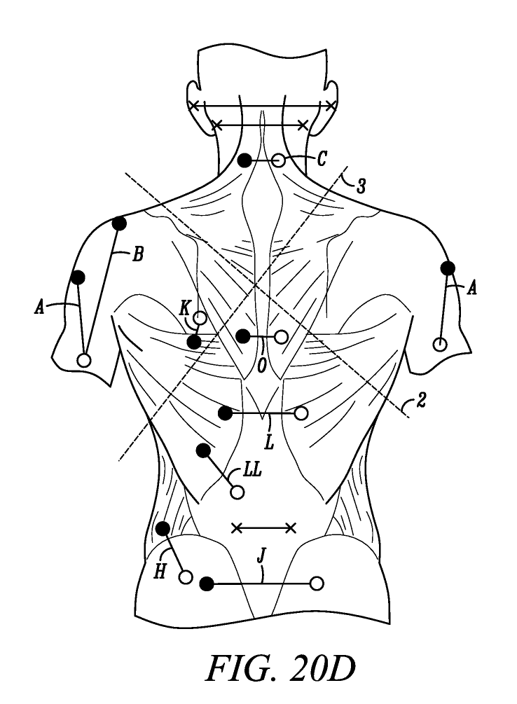

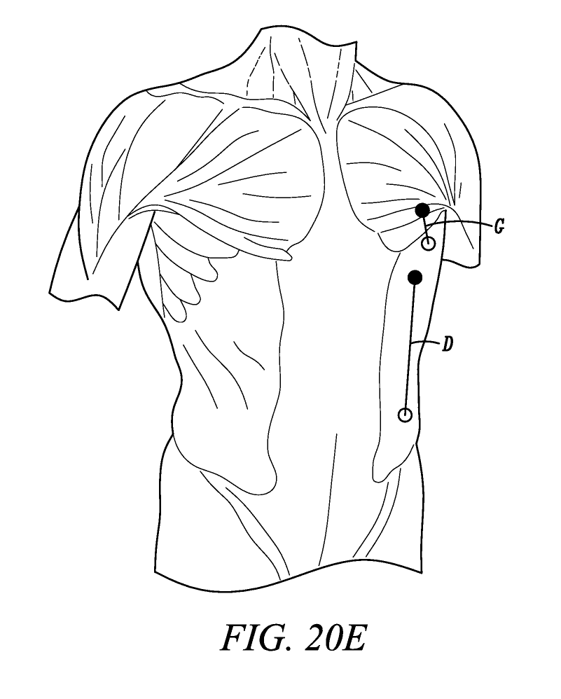

In another embodiment, pairs of locations on or near the right arm for placing electrodes to detect an ECG signal are identified. Referring to FIGS. 20A and 20B, the base of the trapezius 1935, pectoralis 2040 and deltoid 2020 are locations that are related to the electropotential of the second aspect of the ECG signal, meaning that those locations are at a potential related to the heart's conventionally defined right side action potential. Tricep 1910, especially the lateral head area thereof, and bicep 2045 are locations that are related to the electropotential of a first aspect of the ECG signal, meaning that those locations are at a potential related to the heart's conventionally defined left side action potential, even though those locations are in quadrant III. Thus, as was the case with the left arm embodiment described above, by placing one electrode on the tricep 10 and a second electrode on the deltoid 1920, it is possible to detect the action potential of the heart and thus an ECG signal. Again, while certain specific preferred locations on or near the right arm have been described herein as being related to the electropotential of the first aspect of the ECG signal, it should be appreciated that those locations are merely exemplary and that other locations on or near the right arm that are related to the electropotential of the first aspect of the ECG signal may also be identified by making potential measurements.

Referring now to FIGS. 20C, 20D and 20E, a series of electrode pair locations are illustrated. In FIGS. 30C and 20D, the conventionally defined sagittal plane 2 and transverse plane 3 are shown in chain line generally bisecting the torso. Each of the operative pairs are identified, as in FIG. 19C by solid and white dots and chain line. Inoperative pairs are illustrated by X indicators and chain line. As previously stated, inoperative pairs are illustrated to indicate that mere random selection of locations, or selection of independent muscle or muscle groups is insufficient to locate an operative pair of locations. The specific locations identified as within the known operative and preferred embodiments are identified in Table 4 as follows:

TABLE 4

| Reference Letter | First Location (White) | Second Location (Solid) |
|---|---|---|
| A | Tricep | Deltoid |
| B | Tricep | Deltoid (top) |
| C | Right Trapezius | Left Trapezius |
| D | Lower External Oblique | Upper External Oblique |
| E | Upper External Oblique | Lower Pectoralis |
| F | Latissimus Dorsi | Upper External Oblique |
| G | Upper External Oblique | Upper External Oblique |
| H | Gluteus Maximus | Lower External Oblique |
| I | Inguinal Ligament | Lower External Oblique |
| J | Lower Lateral Oblique | Rectus Femoris |
| JJ | Inguinal Ligament | Rectus Femoris |
| K | Rhomboid Major | Latissimus Dorsi |
| L | Latissimus Dorsi | Latissimus Dorsi |
| LL | Thoracumbular Fascia | Latissimus Dorsi |
| M | Left Pectoralis | Deltoid |
| N | Latissimus Dorsi | Upper External Oblique |
| O | Lower Trapezius Right | Lower Trapezius Left |
| P | Pectoralis Left | Pectoralis Left |
| Q | Right Thigh | Left Thigh |
| R | Right Bicep | Right Pectoralis |
| S | Right Inguinal Ligament | Left External Oblique |
| T | Upper External Oblique | Left Arm |
| U | Gluteus Maximus Right | Gluteus Maximus Left |

Similarly, it should be understood that the present invention is not limited to placement of pairs of electrodes on the left arm or the right arm for measurement of ECG from within quadrants I or III, as such locations are merely intended to be exemplary. Instead, it is possible to locate other locations within a single quadrant. Such locations may include, without limitation, pairs of locations on the neck, chest side and pelvic regions, as previously described, that are inequipotential with respect to one another Thus, the present invention should not be viewed as being limited to any particular location, but instead has application to any two inequipotential locations within a single quadrant.

One of the primary challenges in the detection of these signals is the relatively small amplitudes or differences between the two locations. Additionally, these low amplitude signals are more significantly masked and/or distorted by the electrical noise produced by a moving body, as well as the noise produced by the device itself. Noise, in this context, refers to both contact noise created by such movement and interaction of the body and device, as well as electronic noise detected as part of the signal reaching the sensors. An important consideration for eliminating noise is increasing the differentiation between the desired signal and the noise. One method involves increasing signal strength by extending one sensor or sensor array beyond the arm, to the chest or just past the shoulder joint. Consideration must be given with sensor placement to two competing desirable outcomes: increased signal strength/differentiation and compactness of the sensor array or footprint. The compactness is, of course, closely related to the ultimate size of the device which houses or supports the sensors. Alternative embodiments, as described more particularly herein, include arrangements of sensors which strive to maintain a compact housing for the device while increasing distance between the sensors by incorporating a fly-lead going to a sensor location point located some short distance from the device itself, such as on the left shoulder, which is still within quadrant I, or even to the other arm. The system further includes an electronic amplification circuit to address the low amplitude signal.

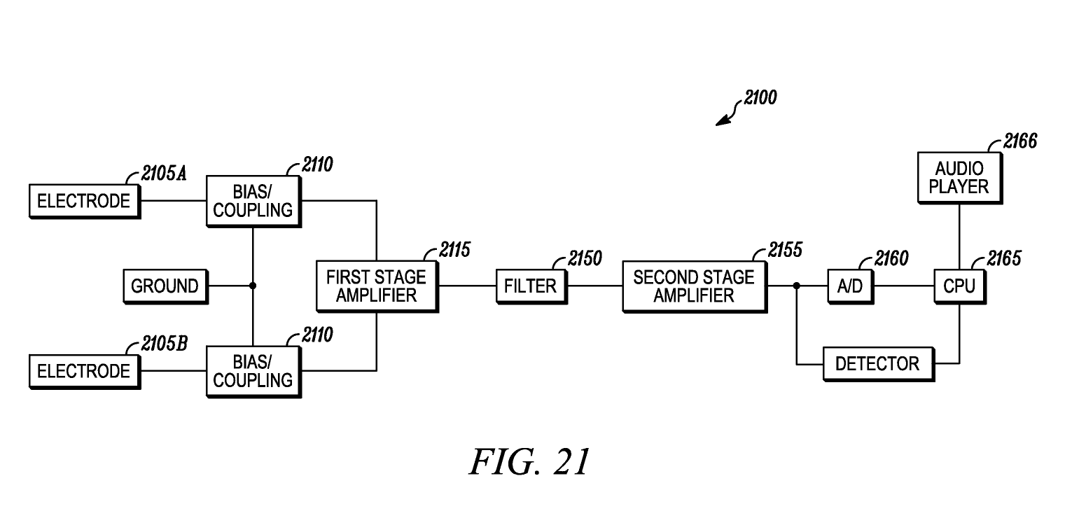

Referring to FIG. 21, a block diagram of circuit 2100 for detecting an ECG signal and for calculating other heart parameters such as heart rate therefrom is shown. Circuit 2100 may be implemented and embodied in a wearable body monitoring device such as the armband body monitoring device described in U.S. Pat. No. 6,605,038 and U.S. application Ser. No. 10/682,293, owned by the assignee of the present invention, the disclosures of which are incorporated herein by reference. Addressing FIG. 21 from left to right, circuit 2100 includes electrodes 2105A and 2105B, one of which is connected to a location as described herein that is related to the electropotential of the first aspect of the ECG signal, the other of which is connected to a location on the body that is related to the electropotential of the second aspect of the ECG signal, even if electrodes 2105A and 2105B are placed within a single quadrant. The interface between the skin and first stage amplifier 2115 is critical as this determines how well the heart rate signal is detected. Electrode contact impedance and galvanic potential are important design consideration when designing the first stage amplifier block and the associated bias/coupling networks.

Electrodes 2105A and 2105B are held against the skin to sense the relatively small voltages, in this case on the order of 20 μV, indicative of heart muscle activity. Suitable electrodes include Red Dot™ adhesive electrodes sold by 3M, which are disposable, one-time use electrodes, or known reusable electrodes made of, for example, stainless steel, conductive carbonized rubber, or some other conductive substrate, such as certain products from Advanced Bioelectric in Canada. It should be noted that unlike the Advanced Bioelectric development, most current reusable electrodes typically have higher coupling impedances that can impact the performance of circuit 2100. Thus, to counteract this problem, a gel or lotion, such as Buh-Bump, manufactured by Get Rhythm, Inc. of Jersey City, N.J., may be used in conjunction with electrodes 2105A and 2105B when placed in contact with the skin to lower the skin's contact impedance. In addition, the electrodes 105 may be provided with a plurality of microneedles for, among other things, enhancing electrical contact with the skin and providing real time access to interstitial fluid in and below the epidermis. Microneedles enhance electrical contact by penetrating the stratum corneum of the skin to reach the epidermis. It is beneficial to make the ECG signal measurements at a position located below the epidermis because, as noted above, the voltages are small, on the order of 20 μV, and the passage of the signal through the epidermis often introduces noise artifacts. Use of microneedles thus provides a better signal to noise ratio for the measured signal and minimizes skin preparation. Such microneedles are well known in the art and may be made of a metal, silicon or plastic material. Prior art microneedles are described in, for example, in U.S. Pat. No. 6,312,612 owned by the Procter and Gamble Company. Based on the particular application, the number, density, length, width at the point or base, distribution and spacing of the microneedles will vary. The microneedles could also be plated for electrical conductivity, hypoallergenic qualities, and even coated biochemically to also probe/sense other physiological electro-chemical signal or parameters while still enhancing the electrical potential for ECG measurement. The microneedles may also be adapted to simultaneously sample the interstitial fluid through channels that communicate with micro level capillary tubes for transferring fluid in the epidermis for sensing electrically, chemically, or electro chemically. Microneedles further enhance the ability of the electrodes to remain properly positioned on the skin during movement of the user. The use of microneedles, however, may limit the ability of the sensors to be mounted on a larger device or housing, as the weight of the larger device may cause the microneedles to shear during movement. In such instances, the microneedle-enhanced sensor may be separately affixed to the body as shown in several embodiments herein. Use of adhesives to supplement the use of microneedles, or alone on a basic sensor is also contemplated. As will be discussed further herein, the use of materials of different flexibilities or incorporating a elastomeric or spring-like responsiveness or memory may further improve sensor contact and locational stability.

In certain circumstances, it is important for a clinician or other observer of the user to determine whether the device has remained in place during the entire time of use, for the purposes of compliance with a protocol or other directive. The use of certain adhesives, or adhesives coupled with plastic or cloth in the nature of an adhesive bandage may be utilized to affix the device to the skin and which would be destroyed or otherwise indicate that removal of the device had occurred or been attempted.

For a wearer to accurately or most affectively place the system on their arm, it may be at least necessary to check that the device is situated in a proper orientation and location, even if the desired location of the electrodes includes an area with significant tolerance with respect to position. In one particular embodiment of the present invention, a device having an array of electrodes 105, such as armband monitoring device 300 described above, is placed in an initial position on the body of the wearer, with each of the electrodes 105 is in an initial body contact position. The device then makes a heart rate or other heart related parameter measurement as described above, and compares the measured signal to a what would be an expected signal measurement for a person having the physical characteristics of the wearer, which had been previously input into the system as more fully described herein, such as height, age, weight and sex. If the measured signal is meaningfully more degraded, as determined by signal to noise ratio or ratio of beat height to noise height, than the expected signal, which would be a preset threshold value, the device gives a signal, such as a haptic, acoustic, visual or other signal, to the wearer to try a new placement position for the device, and thus a new contact position for the electrodes 2105. A second measurement is then made at the new position, and the measured signal is compared to the expected signal. If the measured signal is meaningfully more degraded than the expected signal, the new position signal is given once again to the wearer. This process is repeated until the measured signal is determined by the device to be acceptable. When the measured signal is determined to be acceptable, the device generates a second success signal that instructs the wearer to leave the device in the current placement location. The device may initiate this operation automatically or upon manual request.

Circuit 2100 also includes bias/coupling network 110, shown as two boxes in FIG. 21 for convenience, and first stage amplifier 2115. As will be appreciated by those of skill in the art, the approximately 20 μV potential difference signal that is detected by electrodes 2105A and 2105B will, when detected, be biased too close to the limits of first stage amplifier 2115, described below. Thus, bias/coupling network 2110 is provided to increase the biasing of this signal to bring it within the allowable input range for first stage amplifier 2115.

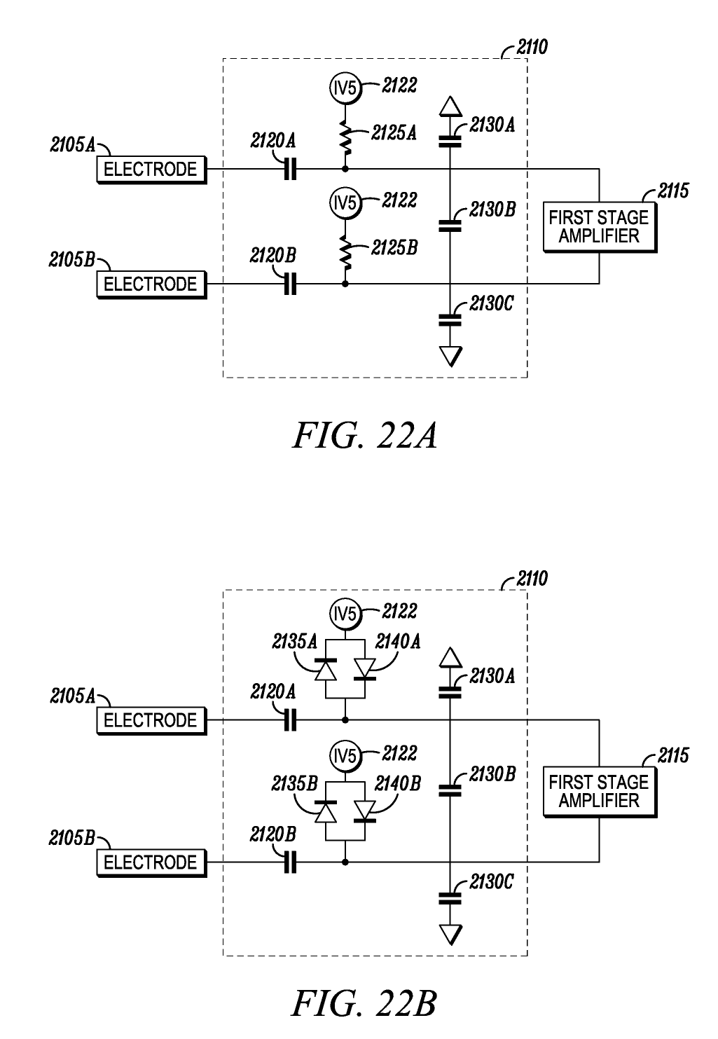

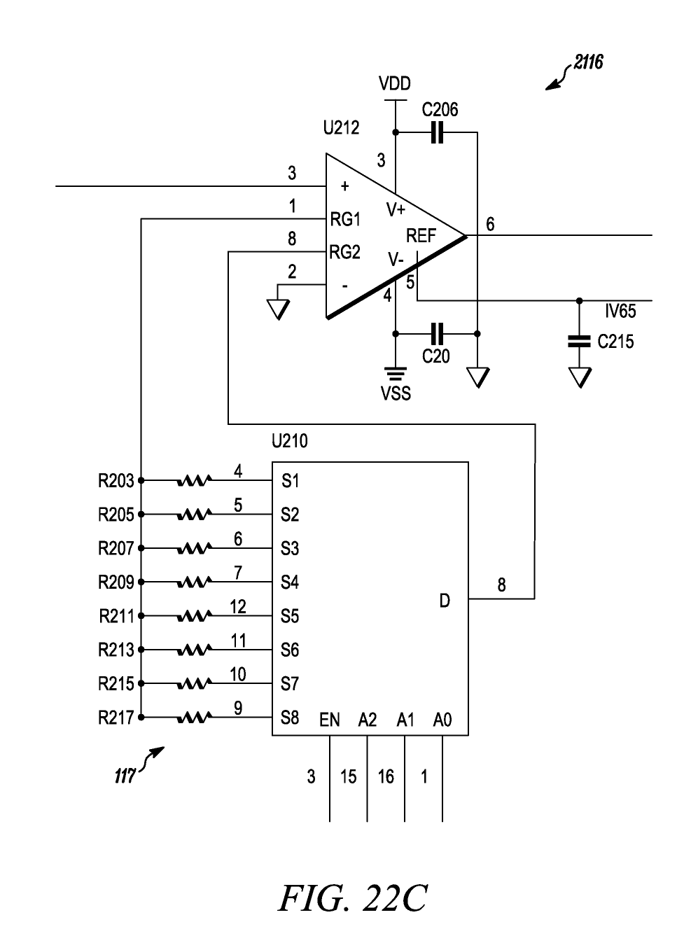

Two approaches to providing bias current for the amplifier inputs are shown in FIGS. 22A and 22B, as will be described more fully herein. Preferably, bias/coupling network 2110 will move the bias of the signal up to the middle range of first stage amplifier 2115. In the preferred embodiment, as described below, first stage amplifier 2115 is a rail to rail amplifier having rails equal to 0 V and 3 V. Thus, bias/coupling network 2110 will preferably increase the bias of the voltage potential difference signal of electrodes 2105A and 2105B to be approximately 1.5 V.

Although not specifically described, the bias/coupling network can be dynamic, in that adjustments can be made based upon the signals being produced when the device is first engaged, or under changing context conditions. This dynamic capability would also accommodate individual differences in amplitude for different placements of similar devices because of user size or other physical characteristics. Experimentation has shown some degree of variation on signal strength based upon distance. Furthermore, changes in signal are expected based on the amount of motion the device is doing relative to the arm, the flexing of the electrodes and their contact with the skin, contractions and relaxations of the muscles below or around the skin contact points and the movement of the body.

Preferably, bias/coupling network 2110 employs capacitive input coupling to remove any galvanic potential (DC voltage) across electrodes 2105A and 2105B when placed on the body that would force the output of first stage amplifier 2115 outside of its useful operating range. In addition, the non-zero input bias current of first stage amplifier 115 requires a current source/sink to prevent the inputs from floating to the power supply rails. In one embodiment, bias/coupling network 2110 may take the form shown in FIG. 22A. In the embodiment shown in FIG. 22A, bias-coupling network 2110 includes capacitors 2120A and 2120B connected to electrodes 2105A and 2105B, respectively, which are in the range of 0.1 μF to 1.0 μF and resistors 2125A and 2125B connected as shown, which have a value of between 2 MΩ to 20 MΩ. As will be appreciated, resistors 2125A and 2125B provide the bias current for first stage amplifier 2115 following Ohm's law, V=IR. In addition, bias/coupling network 2110 includes capacitors 2130A, 2130B and 2130C, the purpose of which is to filter out ambient RF that may couple to the high impedance lines prior to the amplifier in the circuit. Preferably, capacitors 2130A, 2130B and 2130C are on the order of 1000 pF. A 1.5 volt mid-supply reference voltage 2122 is further provided to keep the signals centered in the useful input range of the amplifiers.

Referring to FIG. 22B, an alternative embodiment of bias/coupling network 2110 is shown in which resistors 2125A and 2125B have each been replaced with two diodes connected back to back, shown as diodes 2135A and 2140A and 2135B and 2140B, respectively. In this configuration, with no input signal applied from electrodes 2105A and 2105B, diodes 2135A, 2135B, 2140A and 2140B provide the currents required by first stage amplifier 115 and bias each input slightly away from the 1.5 V reference 2122. When a signal is applied to electrodes 105A and 2105B, the very small change in voltage, typically 20 μV, results in very small changes in current through the diodes, thereby maintaining a high input impedance. This configuration permits exponentially higher currents to bias first stage amplifier 2115 quickly when a large adjustment is necessary, such as is the case during initial application of electrodes 2105A and 2105B to the body. An added benefit of such a configuration is the increased electro-static discharge protection path provided through the diodes to a substantial capacitor (not shown) on the 1.5 V reference voltage 2122. In practice, this capacitor has a value between 4.7 and 10 μF and is capable of absorbing significant electro-static discharges.

Referring again to FIG. 21, the purpose of first stage amplifier 2115 is to amplify the signal received from bias/coupling network 2110 before it is filtered using filter 2150. The main purpose of filter 2150 is to eliminate the ambient 50/60 Hz noise picked up by electrodes 2105A and 2105B when in contact with the body of the user. This noise is often referred to as mains hum. The filter 2150 will add some noise, typically in the range of 1 μV, to the signal that is filtered. Therefore, the purpose of first stage amplifier 2115 is to amplify the signal received from bias/coupling network 2110 before it is filtered using filter 2150 so that any noise added by the filtering process will not overwhelm the signal. As will be appreciated, since the signal output by bias/coupling network 2110 is on the order of 20 μV, filtering with filter 2150 without first amplifying the signal using first stage amplifier 2115 will result in a signal that is overwhelmed by the noise added by filter 2150. Thus, first stage amplifier 2115 is used to amplify the signal with a gain preferably between 100 and 10,000, most preferably 255.

A suitable example of first stage amplifier 2115 is shown in FIG. 22C, which includes programmable gain amplifier 2116, which is preferably model AD627 sold by Analog Devices, Inc. of Norwood, Mass. or model LT1168 sold by Linear Technology Corporation of Milpitas, Calif. The gain of these amplifiers is determined by a gain select resistor coupled to appropriate inputs of the amplifier. Thus, an input multiplexer 2117, such as the model ADG608 multiplexer sold by Analog Devices, Inc. may be used to selectively switch in and out one of a number, preferably 8, of gain select resistors for the programmable gain amplifier used for first stage amplifier 2115 during a testing period to determine an appropriate gain select resistor for the amplifier. Once a candidate gain is determined using the input multiplexer in a testing mode, a single fixed resistor for gain can be selected for use in connection with the programmable gain amplifier used as first stage amplifier 2115.

Key parameters in selecting an amplifier for first stage amplifier 2115 are input bias current, input offset current, and input offset voltage. Input bias current multiplied by the input impedance of the bias/coupling network gives the common-mode input offset voltage of the positive and negative inputs to first stage amplifier 2115. Care must be taken to keep the inputs of first stage amplifier 2115 far enough from the power supply rails to prevent clipping the desired output signal. As with the bias/coupling network, an alternative design might include a circuit which was able to dynamically limit the input voltage based upon the type of activity, such as power on, initial attachment to the arm, or certain high-motion activities so that the input voltage under normal conditions would be optimum. As one skilled in the art would appreciate, some clipping can be acceptable. Algorithms for detecting heart rate or other heart parameters can work in the presence of some amount of clipping, assuming that the signal to noise ratio remains relatively high.

The input offset current parameter multiplied by the bias impedance gives the differential input voltage that is applied to first stage amplifier 2115. This differential voltage is in addition to the input offset voltage parameter that is inherent in the amplifier, and the total input offset is simply the sum of the two. The total differential input voltage multiplied by the gain determines the output offset. Again, care must be taken to keep the output signal far enough from the power supply rails to prevent saturation of the amplifier output. As an example, a bipolar amplifier such as the model AD627 described above has an input bias current of 10 nA, an input offset current maximum of 1 nA, and an input offset voltage of 150 μV (all values are worst case maximums at 25° C.). In order to keep the common-mode input offset to less than 0.5 V, the bias impedance must be no more than 0.5 V/10 nA=50 MΩ. However, the input offset current dictates that in order to maintain a maximum 0.5 V output offset voltage, one must provide an input impedance of no more than 0.5 V/gain/1 nA. For a gain of 100, this resolves to 5 MΩ. For a gain of 500, this resolves to 1 MΩ. Another candidate amplifier for use as first stage amplifier 2115 is the Texas Instruments Model INA321 programmable gain amplifier, which has FET inputs. This amplifier has an input bias current of 10 pA and an input offset current of 10 pA (max). In order to keep the common-mode input offset to less than 0.5 V, one must provide an impedance of no more than 0.5 V/10 pA=50 GΩ. However, the input offset current dictates that in order to maintain a maximum 0.5 V output offset, one must provide an input impedance of no more than 0.5 V/gain/10 pA. For a gain of 100, this resolves to 500 MΩ. For a gain of 1,000, this resolves to 50 MΩ.

As an alternative, as will be appreciated by those of skill in the art, first stage amplifier 2115 may be implemented in a network of low cost discrete op-amps. Such an implementation will likely reduce the cost and power consumption associated with first stage amplifier 2115. As will also be appreciated by those of skill in the art, the same analysis of amplifier input bias current, output saturation, and input bias/coupling applies to such an alternative implementation.

Referring again to FIG. 21, filter 150 is a bandpass network preferably including separate low-pass and high-pass filter sections. The purpose of the low-pass filter section is to eliminate the ambient 50/60 Hz noise picked up by electrodes 2105A and 2105B when in contact with the body. Preferably, a multi-pole filter is used to achieve a high degree of attenuation. The high-pass filter section eliminates the DC wander of the signal baseline due to galvanic effects in electrodes 105A and 105B, allowing the heart beat spikes forming a part of the measured ECG signal to be more easily detected by hardware or software means.

In one embodiment, filter 2150 includes switched capacitor low-pass and high-pass filters with adjustable cutoff frequencies to allow for experimentation. Such a filter 2150 may be constructed using the model LTC1164_{—}6 low-pass filter chip sold by Linear Technology Corporation followed by a model LTC1164 high-pass filter chip also sold by Linear Technology Corporation, which chips provide an eighth order elliptical filter with very sharp cutoff characteristics. Experimentation with this implementation has shown that a low-pass cutoff frequency of 30 Hz and a high-pass cutoff frequency of between 0.1 Hz and 3 Hz worked well. Although allowing for flexibility, this implementation is relatively expensive and was found to consume a significant amount of power.

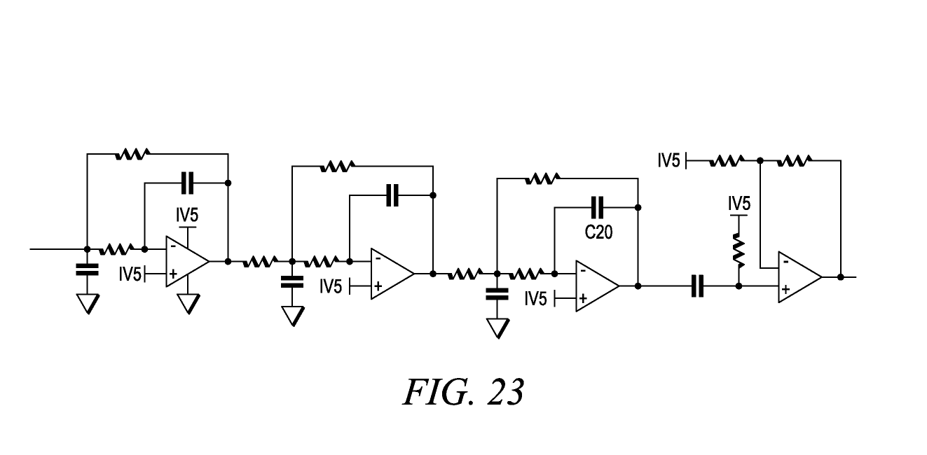

An alternative implementation for filter 2150 is shown in FIG. 23. The circuit shown in FIG. 23 implements a sixth order active filter using discrete op-amps in a multiple feedback topology. The circuit shown in FIG. 23 consumes less current and costs significantly less than the switched capacitor design described above. Values for the resistors and capacitors shown in FIG. 23 may be selected using a software tree package such as the FilterPro package provided by Texas Instruments. As will be appreciated by those of skill in the art, the different filter styles, such as Butterworth, Bessel, and Elliptic, may be implemented simply by changing component values. The FilterPro package also provides information that is useful in selecting the amplifiers shown in FIG. 23, including necessary bandwidth for each stage. Suitable amplifiers include the models TLV2764 and OPA4347 quad amplifiers sold by Texas Instruments Incorporated of Dallas, Tex. The three-stage (first three op-amps) sixth order filter forming part of the circuit shown in FIG. 23 provides adequate 60 Hz filtering, thereby allowing the fourth op-amp in the circuit to be used for second stage amplifier 155 shown in FIG. 21 and described below. In addition, the R-C Network shown in FIG. 21 that couples the third stage op-amp of the low-pass filter to the fourth op-amp (the gain stage) provides a high-pass network which eliminates DC drift as described above.

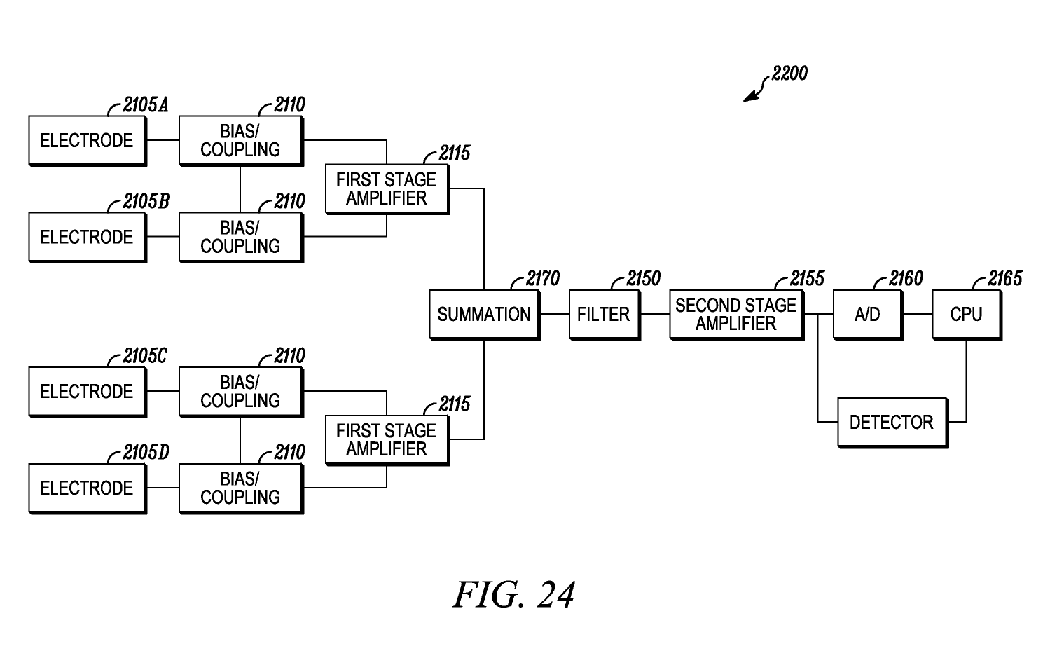

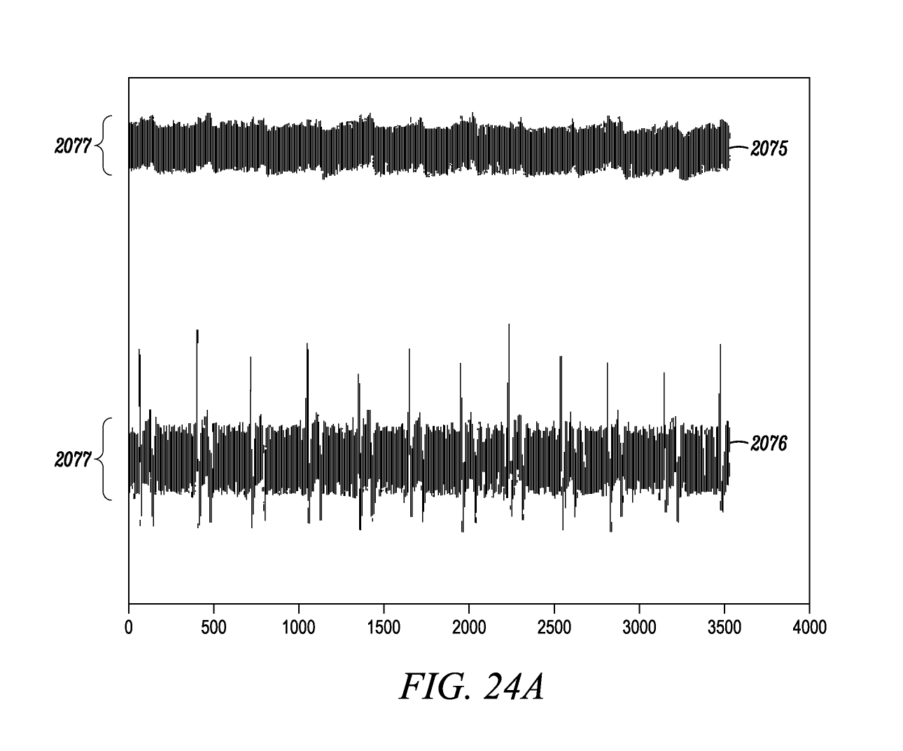

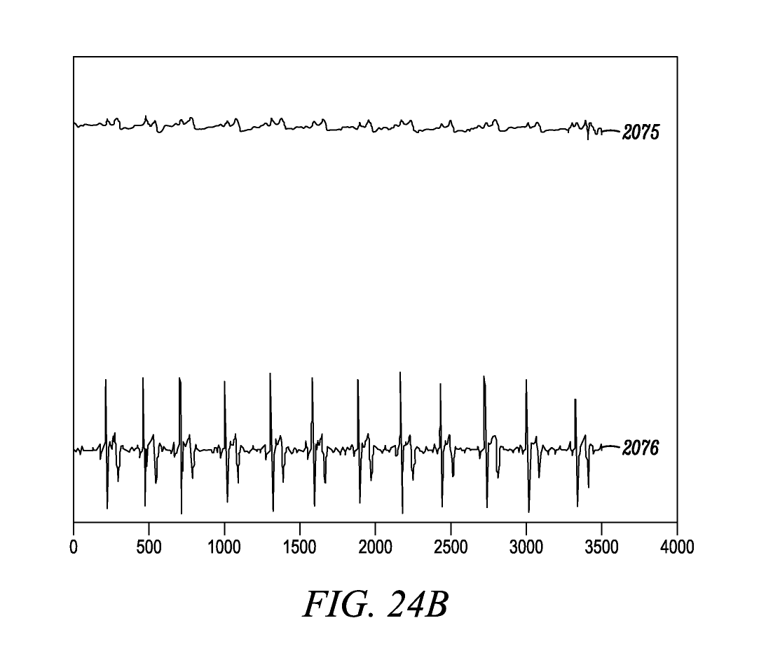

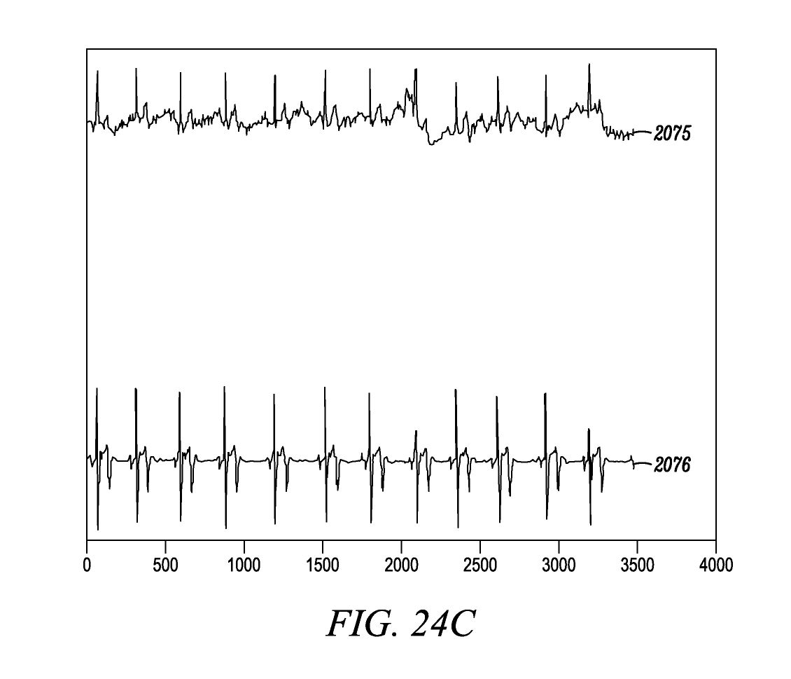

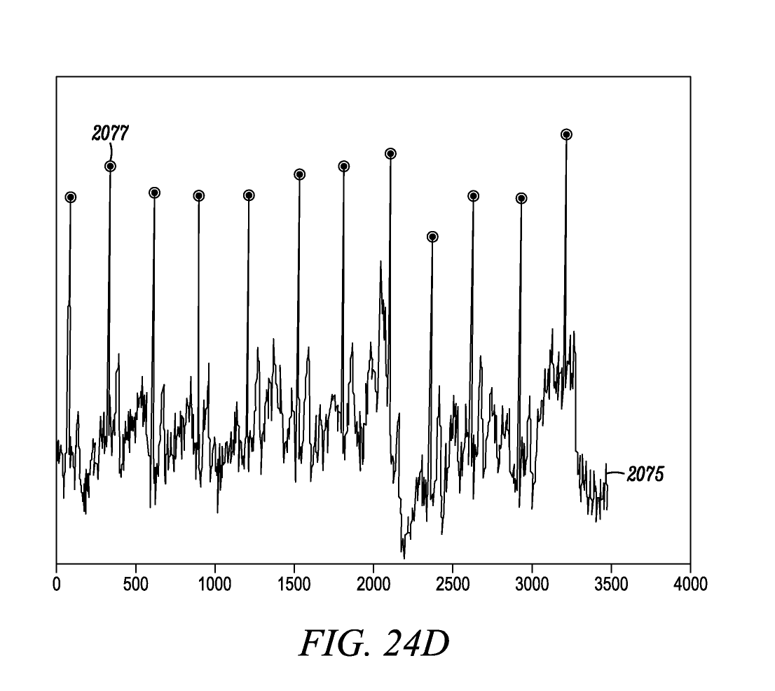

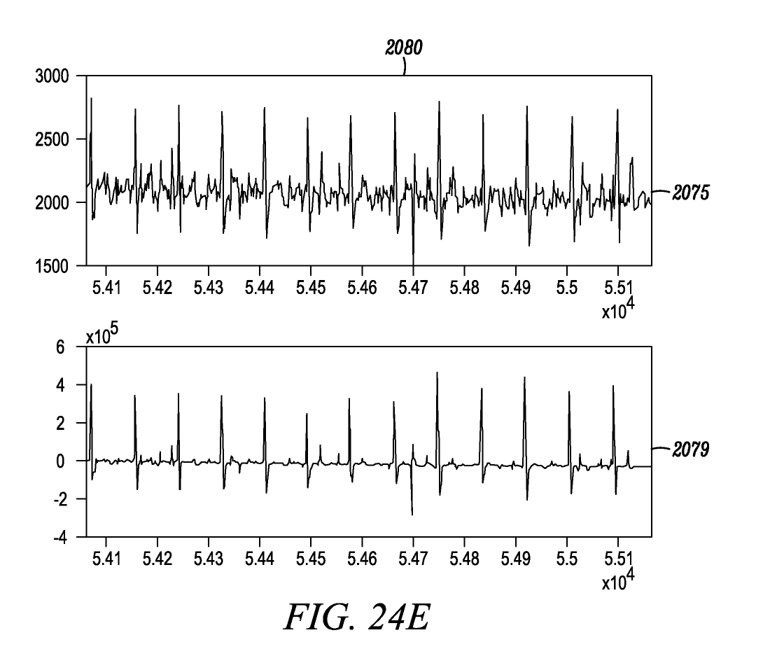

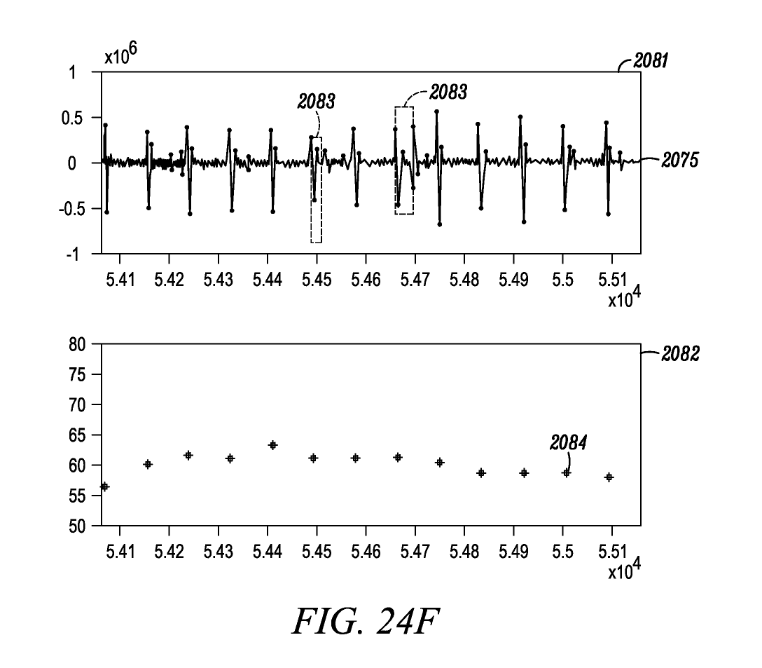

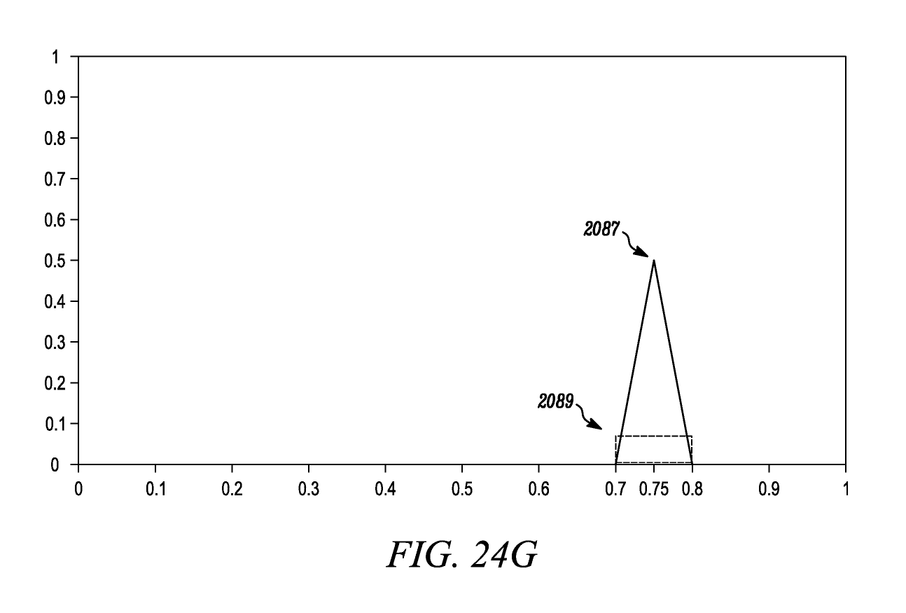

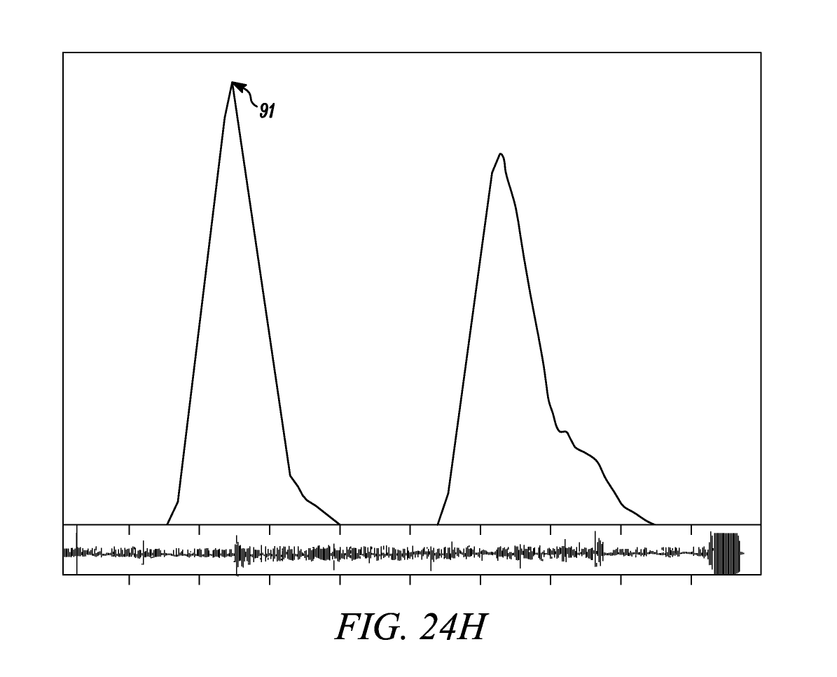

Referring again to FIG. 21, circuit 2100 includes second stage amplifier 2155 for amplifying the signal output by filter 2150 to a level that can be directly sampled by analog to digital converter 2160. Specifically, if the gain of first stage amplifier 2115 is between 100 and 10,000, the amplitude of the signal output by filter 2150 will be in the range of 2 mV to 200 mV. Preferably, the gain of first stage amplifier 2115 is 500, and therefore the amplitude of the signal output by filter 2150 will be on the order of 10 mV. In order to allow for a higher sampling resolution by analog to digital converter 2160, second stage amplifier 2155 is used to further amplify the signal. Preferably, second stage amplifier has a gain on the order of 30, and therefore would amplify the 10 mV signal in the preferred embodiment to a 300 mV signal. However, the gain of second stage amplifier 2155 may also be on the order of 10 to 100. As was the case with first stage amplifier 2115, a programmable gain amplifier may be used for second stage amplifier 2155. Alternatively, as described above, the unused (fourth) op-amp in the filter 150 implementation shown in FIG. 24 may be used for second stage amplifier 2155.

Analog to digital converter 2160 converts the analog waveform output by second stage amplifier 2155 into a digital representation that can then be processed by one or more algorithms, as described more fully herein, to determine heart related parameters, such as heart rate, therefrom. Analog to digital converter 2160 may be implemented using a 12 bit analog to digital converter with a 3 V reference at 32-256 samples per second. Such a device is integrated into the Texas Instruments MSP430F135 processor. Analog to digital converter 2160 is connected to central processing unit 2165, which reads the converted digital signal and performs one of the following functions: (i) it stores the raw digital signal to memory, such as flash or SRAM, for subsequent analysis; (ii) it stores a number of raw digital signals to memory and subsequently transmits them, wired or wirelessly, to a remote computer for analysis as described herein and/or display, such as display in real time; or (iii) it processes the raw digital signals using algorithms described herein provided on central processing unit 2165 to determine heart related parameters, such as the timing and various sizes of heart beats, heart rate, and/or beat-to-beat variability. With respect to this last function, central processing unit 2165 may, once heart beats and/or heart rate has been determined, perform a variety of tasks such as blink an LED for each beat or store heart rate information to memory. Optionally, central processing unit may provide operational control or, at a minimum, selection of an audio player device 2166. As will be apparent to those skilled in the art, audio player 166 is of the type which either stores and plays or plays separately stored audio media. The device may control the output of audio player 2166, as described in more detail below, or may merely furnish a user interface to permit control of audio player 2166 by the wearer.

These functions can also be performed independently in sequence. For example, the data can be stored in real time in a data storage medium while being simultaneously analyzed and output. Subsequent processes can allow the system to retrieve earlier stored data and attempt to retrieve different information utilizing alternative algorithmic techniques or filters. Additionally, data from different points in the filtration process, described above, can be simultaneously stored and compared or individually analyzed to detect signal information which is lost at certain points in the process.

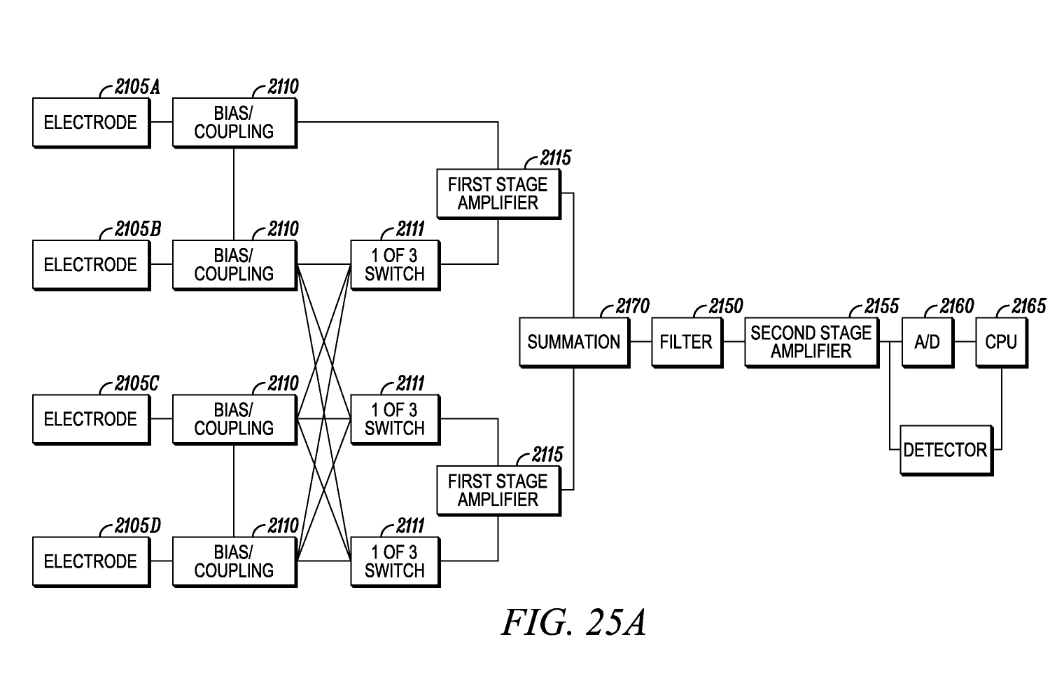

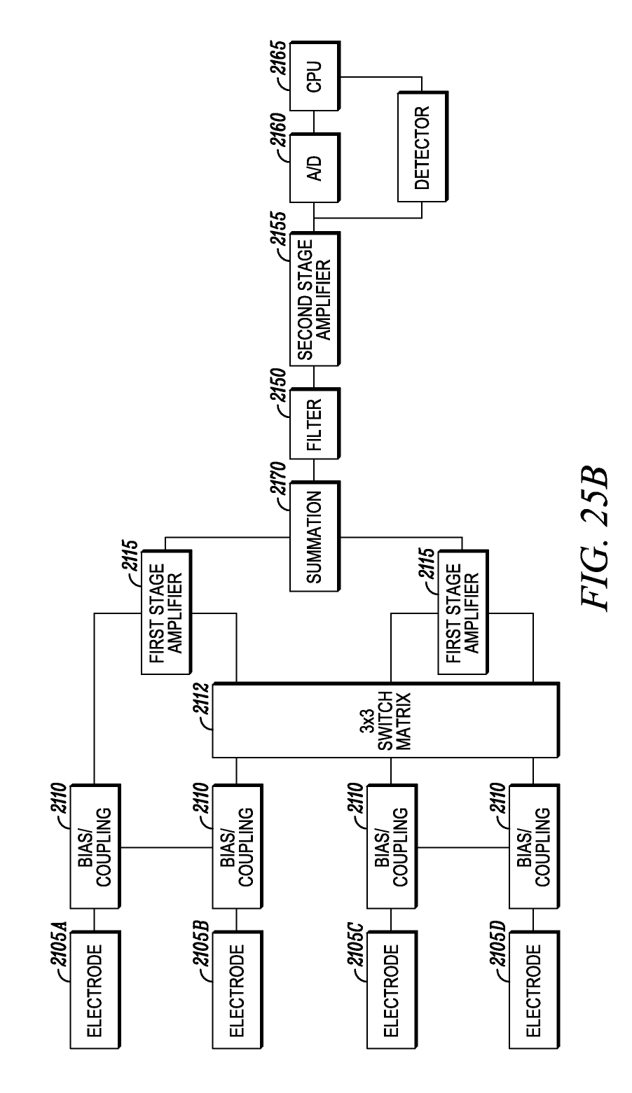

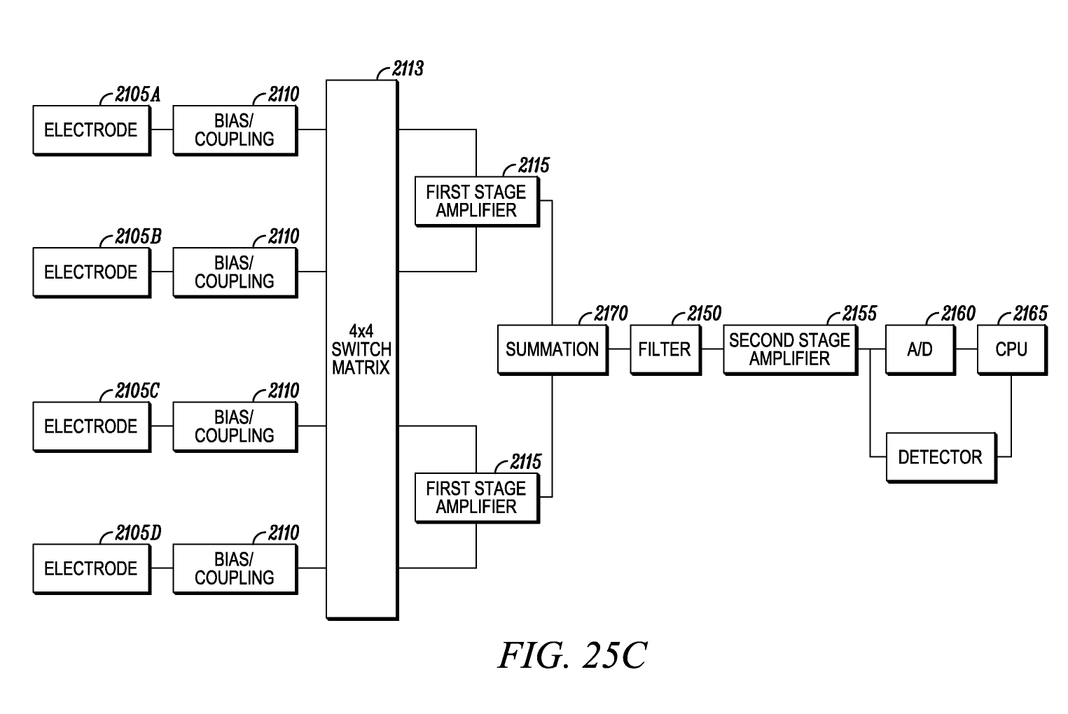

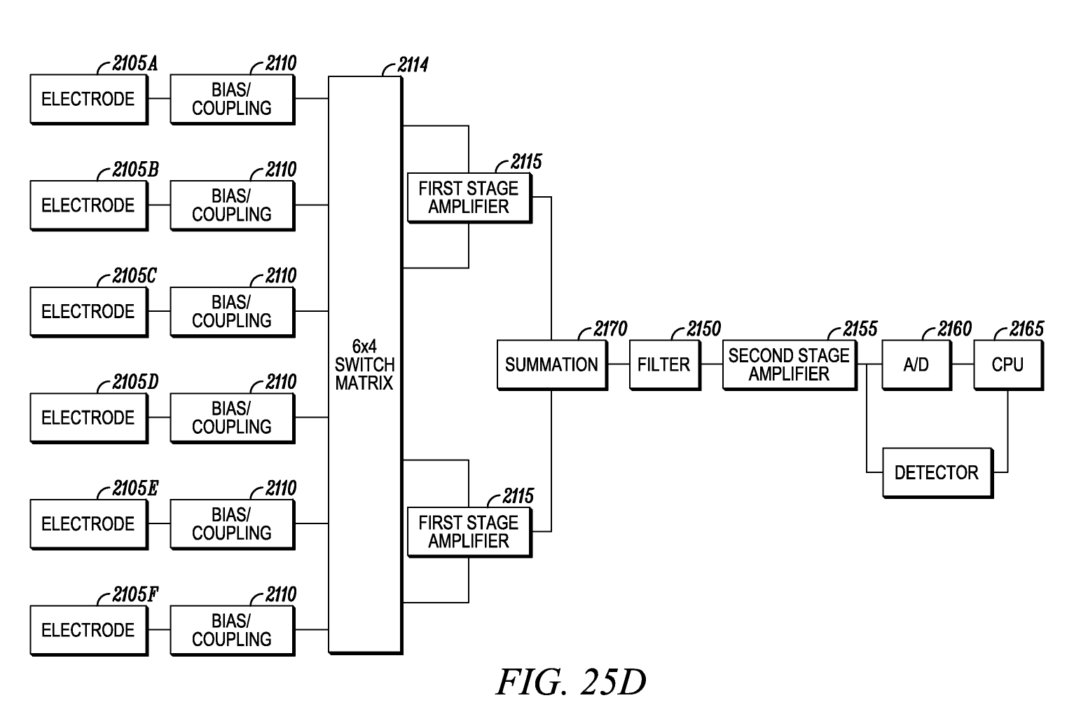

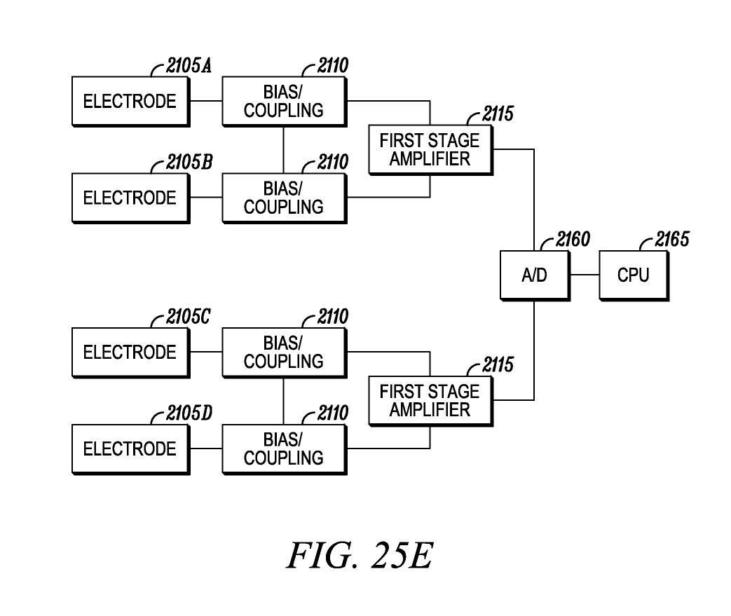

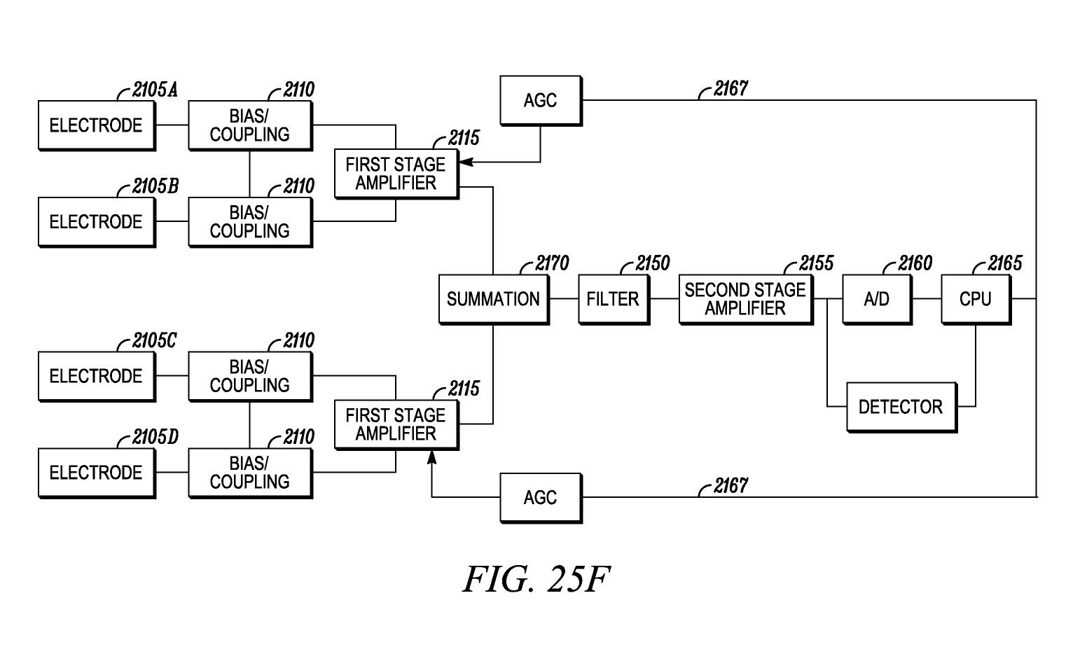

Referring to FIG. 24, alternate circuit 2200 for measuring an ECG signal is shown in which an array of multiple electrodes 2105, for example four electrodes 2105A through 2105D, are used. The electrodes 2105 in this embodiment are grouped in pairs and, as was the case with circuit 2100 shown in FIG. 24, one electrode of each pair is placed in a location that is related to the electropotential of the right side of the ECG signal and the other electrode in each pair is placed in a location that is related to the electropotential of the left side of the ECG signal. The first electrodes in each pair may be placed in locations close to one another to attempt to get a good signal form a particular general location, or may be placed in locations removed from one another, as illustrated in the particular embodiments described with more detail below, to pick up signals from different locations. The second electrodes in each pair may be similarly placed. Each pair of electrodes 2105 is connected to bias/coupling network 110 as described above, and the output is connected to a first stage amplifier 2115 as described above. In the embodiment shown in FIGS. 24, 25A-D and 25F, the output of each first stage amplifier 2115 is fed into summation circuit 2170, which for example may be a resistor network. Summation circuit 2170 adds the outputs of the first stage amplifiers 2115 together. The summed signal is then passed through filter 2150, second stage amplifier 2155, and to analog to digital converter 2160 and central processing unit 2165 as described above.

It is to be specifically noted that the circuitry may be implemented in a minimal cost and component embodiment, which may be most applicable to a disposable application of the device. In this embodiment, the apparatus is not provided with a processor, only electrically separated electrodes for picking up a voltage difference, a gating mechanism for differentially passing current associated with voltage spikes, such as QRS signals and a mechanism for displaying characteristics of the passed through current. This apparatus may be powered by motion, battery, or solar power. Another option is to power the apparatus directly from the voltage potentials being measured. The display mechanism may be chemical, LCD or other low power consumption device. The voltage spikes charge up a capacitor with a very slow trickle release; a simple LED display shows off the charge in the capacitor. In another embodiment, a simple analog display is powered by the battery. The simple apparatus utilizes digital processing but no explicit processor; instead a simple collection of gates, threshold circuitry and accumulator circuitry, as would be apparent to one skilled in the art, based upon the descriptions above, controls the necessary preprogrammed logic.

The implementation shown in FIGS. 24 and 25A-F, which utilize an array of electrodes 2105, is particularly useful and advantageous due to the fact that the signals detected by electrodes 2105 can at times be saturated by muscle activity of the body, such as muscle activity in the arm in an embodiment where electrodes 2105 are placed on locations of the arm. The heart beat related portion of the signals detected by electrodes 2105 are coherent, meaning highly correlated, while the muscle activity noise portions of the signals tend to be incoherent, meaning not correlated. Thus, because of this coherent/incoherent nature of the different portions of signals, when the signals generated by electrodes 2105 are summed, subtracted, averaged, multiplied or the like, by summation circuit 2170, the heart beat related components will add to one another thereby producing better heart beat spikes having a higher signal to noise ratio, while the muscle noise related components will tend to wash or cancel one another out because the “hills” and “valleys” in those signals tend to be off phase from one another. The result is a stronger heart beat related signal with less muscle related noise.

FIGS. 25A through 25F illustrate alternative embodiments of the system incorporating multiple electrodes shown in FIG. 24. FIG. 25A illustrates, three electrodes 2105B-F interchangeably routed by switches 2111 to any of the first stage differential amplifier 2115 inputs to allow various combinations of electrode subtractions and additions. This arrangement assumes that one electrode will always be treated in the positive sense. FIG. 25B illustrates an arrangement similar to FIG. 25A, however, a 3×3 switch matrix 2112 is utilized rather than the discrete switches shown in FIG. 25A. FIG. 25C illustrates a 4×4 switch matrix 2113, which allows full control of electrode pair addition/subtraction and is the most simple conceptually. In some embodiments, the functionality of the switch matrix 113 may be reduced to permit only certain pairings in order to obtain a cleaner signal. FIG. 25D illustrates a 6×4 switch matrix 2114, which allows full control of electrode pair addition/subtraction and permits the selection of two pairs from the full suite of electrodes. FIG. 25D includes additional electrodes 2105E-F to illustrate the selectability of three full pairs of such electrodes. As with the embodiment shown in FIG. 25C, the functionality of the switch may be reduced to permit only certain pairings. This could conceptually be expanded to as many electrodes as desired. FIG. 25E illustrates an embodiment that provides electrode shielding, and the individual pairs of electrodes can be sampled and then summed and/or subtracted during subsequent analysis, the strongest pair may simply be chosen or the average may be taken of an array of signals. This arrangement can also require 50-60 Hz filtering and higher first stage amplifier gains to keep the signal to noise ratio high. FIG. 25F illustrates an embodiment in which the CPU controls the gain of the first stage amplifier through AGC circuits 2167, enabling the system to adjust for poor electrode placement or subjects with weaker ECG signals. These embodiments permit the selection of the strongest pair or best signal from of a multiplicity of pairs of electrodes for analysis. This can be accomplished according to several methodologies in addition to mere signal strength. These include the analysis of all the pairs and combination of the signals or calculation of an average of all of the signals or the identification of the most distorted signal, considering muscle artifact noise or the like, and utilizing it as a filter signal to be subtracted from the identified best signal.

There are multiple sources of noise that can affect the amplified signal that is input into analog to digital converter 2160 shown in FIGS. 21, 24 and 25A-F. For example, as described above, mains hum and DC wander noise can effect the signal. In the embodiments shown in FIGS. 21, 24 and 25A-F, this noise is removed using filter 2150. In an alternate embodiment, rather than using a hardware solution like filter 2150 to remove the 50/60 Hz mains hum and/or DC wander noise from the voltage potential difference signal received from electrodes 2105, some or all of this noise can be filtered out of the signal, after being digitized by analog to digital converter 2160, using known software techniques implemented in software residing either on CPU 2165 forming a part of a body monitoring device or on a separate computer that receives the digitized signal. In this embodiment, filter 2150 would be eliminated and only a single amplifier having a gain on the order of 500 to 2500 such as first stage amplifier 2115 would be used in circuit 2100 or 2200. A two stage amplifier may also be utilized, having first stage gain of 50-500 and a second stage gain of 10-50. These steps (in either the hardware or software implementations), in effect remove components of the signal having frequencies that are considered to be too high or too low to constitute a heart related signal, with a typical ECG signal having a frequency in the range of 0.5-4 Hz.

The system is specifically designed to minimize the processing time delays and interruptions created by noise being processed and subtracted or filtered from the primary signal. As noise is processed and consuming processor resources, data must be stored and processed at a later time. It is important to return as quickly as possible to contemporaneous monitoring so as to avoid the build up of a backlog of data. The system utilizes a plurality of measurement techniques, such as described above to quickly identify and extract the primary signal and rapidly return to real time monitoring. Most particularly, the circuitry is designed to minimize DC wander within three beats of the heart.

In addition, another source of noise that may affect the signal input into analog to digital converter 2160 is muscle noise caused by the electrical activity of muscles. Electromyography, or EMG, is a measurement of the electrical activity within muscle fibers, which is generally measured actively, but could also be measured passively, according to the method of subtraction or filtering of the most distorted signal described above, because it is affected most by muscle artifact and/or has very little if not any signal relating to the heart related electrical activity. While a subject is in motion, electrodes 2105 for measuring ECG may also simultaneously pick up and measure EMG signals. Such contemporaneously measured EMG signals are noise to the ECG signal. Thus, according to an aspect of the present invention, ECG signal measurement can be improved by using separate electrodes to specifically measure an EMG signal, preferably from body locations that have a minimal or difficult to detect ECG signal. This separately measured EMG signal may then be used to reduce or eliminate EMG noise present in the separately and contemporaneously measured ECG signal using various signal processing techniques. In many cases, the EMG signal's amplitude may so overwhelm that ECG signal that either filtering or utilizing the above-described method may not result in a usable ECG signal. In these events, the use of a non-electrode sensor could be utilized in conjunction with electrodes in order to detect the relatively quiet ECG signal. This sensor may even replace the beat detection if it detected ECG peaks when the primary electrical signal clips, gets oversaturated or overwhelmed by the EMG signal. An example sensor is a micro-Doppler system, either as a single pick-up or an array, designed to pick up the mechanical rushing of blood or the like, past the Doppler signal, creating a pulse wave in which the peak could be recognized and timed as a beat. This embodiment could be tuned to a specific location or utilize an array of different sensors tuned to different depths in order to optimize and locate the best signal for each user. This array could also be utilized, through monitoring of different signals and signal strength, to locate the device at the best position on the arm through well known audible or visual feedback mechanisms. The device could also be tuned to certain individual characteristics detected over an introductory period of evaluation or tuned dynamically over a period of time. Under certain high noise circumstances, the mechanical signal might be substituted for the electrical ECG signal as part of the calculations. In order to make the mechanical and electrical wave align, timing and phase shift differences would have to be calculated and factored into the peak or beat recognition algorithm. This system could be also utilized for detection and measurement of pulse transit time, or PTT, of the wearer, as described more fully herein, allowing relative and/or absolute measurement of blood pressure could be derived or calculated.

Pulse transit time, or PTT, is the time that it takes a pulse pressure waveform created by a heart beat to propagate through a given length of the arterial system. The pulse pressure waveform results from the ejection of blood from the left ventricle of the heart and moves through the arterial system with a velocity that is greater than the forward movement of the blood itself, with the waveform traveling along the arteries ahead of the blood. PTT can be determined by measuring the time delay between the peak of a heart beat, detected using the R-wave of an ECG signal and the arrival of the corresponding pressure wave at a location on the body such as the finger, arm, or toe, measured by a device such as a pulse oximeter or other type of pressure detector. As blood pressure increases, more pressure is exerted by the arterial walls and the velocity of the pulse pressure waveform increases. The velocity of the pulse pressure waveform depends on the tension of the arterial walls; The more rigid or contracted the arterial wall, the faster the wave velocity. As a result, for a fixed arterial vessel distance, as PTT increases and pulse pressure waveform velocity decreases, blood pressure decreases, and as PTT decreases and pulse pressure waveform velocity increases, blood pressure increases. Thus, PTT can be measured and used to indicate sudden changes in real-time blood pressure.

In one embodiment, the same armband device includes the ability to detect the ECG signal and in conjunction with a micro Doppler array against the body, together create the PTT measurement. An aspect of the present invention relates to the measurement and monitoring of PTT. Specifically, the time of a heart beat peak can be determined using an ECG signal using electrodes 105 as described herein. The time of the arrival of the corresponding pressure wave at a given location on the body can be measured using any one of a number of pressure sensors. Such pressure sensors may include, but are not limited to, pulse oximeters, Doppler arrays, single piezoelectric sensors, acoustic piezoelectric sensors, fiber optic acoustic sensors, blood volume pressure or BVP sensors, optical plethysmographic sensors, micropower impulse radar detectors, and seismophones. According to a preferred embodiment of the present invention, PTT is measured and monitored to indicate changes in blood pressure using armband body monitoring device 300 that is provided with one or more of the pressure sensors described above. Thus, in this embodiment, PTT is measured in a single device that obtains an ECG signal from the upper arm and that measures the arrival of the pulse pressure waveform at a location on the upper arm. Alternatively, the pressure sensor may be located separately from armband body monitoring device 300 at a different location, such as the finger or wrist, with the information relating to the arrival time being transmitted to armband body monitoring device 300 for calculation. This calculation may also be made at the finger product, or other third product, or shared between any combination of the above. Communication between each device can be provided in a wired or wireless embodiment, or transmitted through the skin of the wearer, as is well known to those skilled in the art.

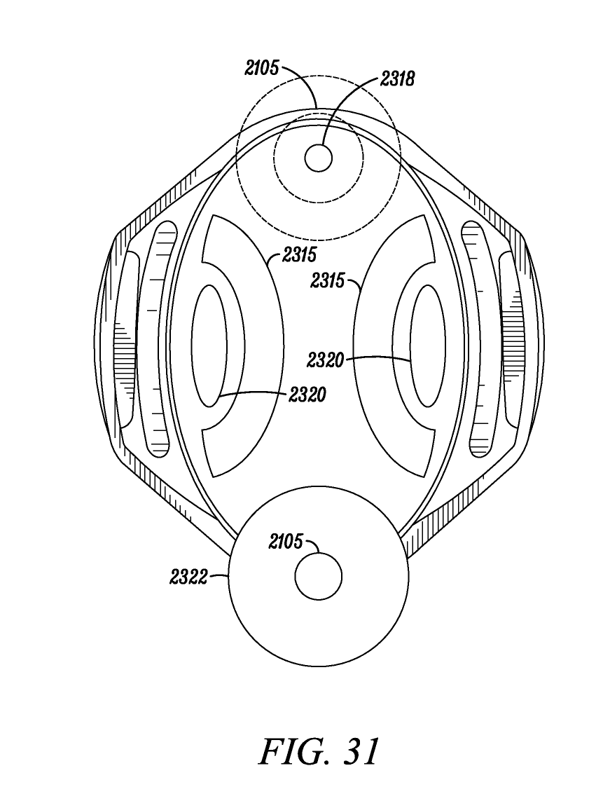

In one specific embodiment, electrodes 2105 may be placed on the deltoid muscle and the triceps muscle of the left arm in order to measure an ECG signal, which will likely contain muscle related noise, and separate electrodes 2105 may be placed one each on the triceps muscle or one on the triceps muscle and one on the brachialis muscle for collecting an EMG signal having little or no ECG component, according to at least one of the several embodiments of the device more fully described below. This EMG signal may then be used to process and refine the measured ECG signal to remove the EMG noise as described herein. An example of such a configuration is armband body monitoring device 300 described below in connection with the specific alternative embodiments of the device, and more specifically FIG. 31, in which electrodes 2105A and 2105B would measure an ECG signal likely containing muscle related noise, and electrodes 2105C and 2105D measure an EMG signal having little or no ECG component.

Although muscle noise can be reduced using separate EMG sensors as just described, it has been found that this noise, to a degree, often ends up remaining in the signal input into analog to digital converter 2160 despite efforts to eliminate or reduce such noise. The amplitude of actual heart beat spikes, which comprise the QRS wave portion of the ECG signal, in the collected signal may vary throughout the signal, and the remaining muscle noise may obscure a heart beat spike in the signal or may itself look like one or more heart beat spikes. Thus, an aspect of the present invention relates to various processes and techniques, implemented in software, for identifying and reducing noise that is present in the digital signal output by analog to digital converter 2160 and identifying heart beats and heart beat patterns from that signal. In addition, there may be portions of the signal that, despite processing efforts, contain too much noise and therefore no discernable heart related signal. A further aspect of the present invention relates to process and techniques for dealing with such portions and interpolating the data necessary to provide continuous and accurate output.

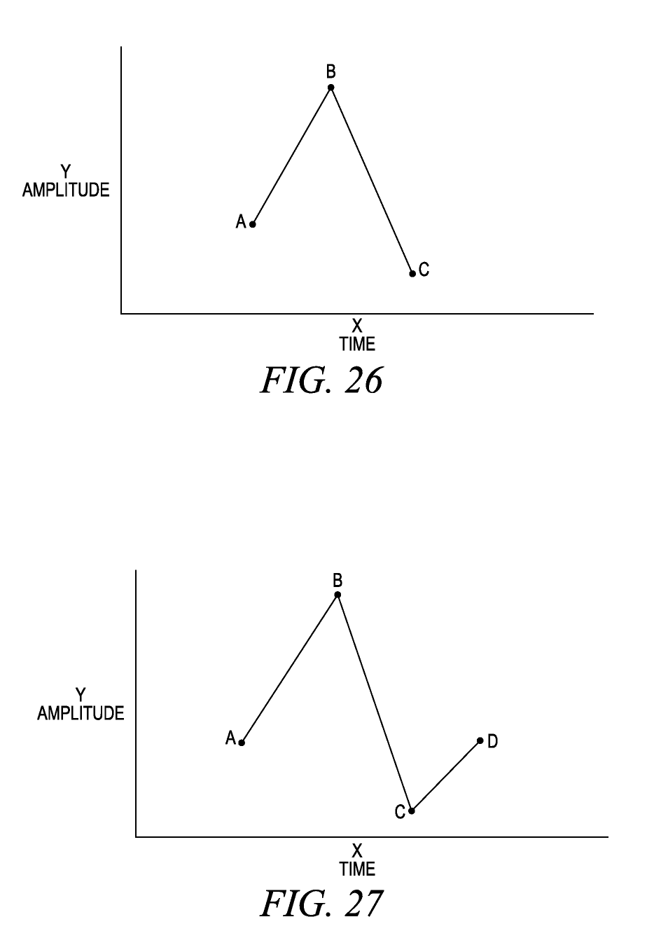

According to a one embodiment of the present invention, the signal that is output by analog to digital converter 2160 may first undergo one or more noise reduction steps using software residing on either CPU 2165 or on a separate computer to which the signal has been sent. For example, in one possible noise reduction implementation, the signal is first processed to identify each peak in the signal, meaning an increasing amplitude portion followed by a maximum amplitude portion followed by a decreasing amplitude portion. An example of such a peak is shown in FIG. 26 and includes points A, B and C wherein the X axis is time and the Y axis is signal strength or amplitude. For each identified peak, the height of the peak (in units of amplitude) and the width of the peak (in units of time) are then calculated. Preferably, the height for each peak is determined as follows: min (B_{Y}−A_{Y}, B_{Y}−C_{Y}), and the width for each peak is determined as follows: (C_{X}−A_{X}). In addition, a standard height and width profile of a heart beat spike, comprising the QRS wave, is established and stored, and identified peaks present in the signal that are outside of the stored profile are eliminated, meaning that those portions of the signal are marked to be ignored by further processing steps because they constitute noise. In a preferred embodiment, the standard height in the stored profile is approximately 400 points when a 128 Hz analog to digital sampling rate is used and a 12-bit encoding of the signal is used and the standard width in the stored profile is approximately 3 to 15 points when a 128 Hz analog to digital sampling rate is used and a 12-bit encoding of the signal is used. In one particular embodiment, the profile may constitute an adaptive height and/or width that is stored and used for identifying spikes in the signal that are to be eliminated, such as a height and/or width based on a percentage of the moving average of previous measurements. In addition, peaks in the signal that hit the maximum and minimum value rails output by analog to digital converter 160 may be eliminated as well. Peaks may also be eliminated from the signal if they would indicate an unlikely heart rate given the surrounding signal context, i.e., other peaks in close proximity that would result in a calculated heart rate that is above a likely maximum value. Finally, noise can be removed based on using additional sensors preferably provided with the body monitoring device that implements circuit 100 shown in FIG. 21 or circuit 2200 shown in FIG. 24, including, but not limited to, accelerometers or other motion detecting sensors for detecting either motion or tension, audio sensors, or using time-spectrum signature of muscle noise.

FIGS. 24A through 24D illustrate the progressive steps of obtaining and extracting the ECG data and heart beats from the detected signal. Referring now to FIG. 24A, the detected signal 2075 is illustrated in conjunction with a simultaneously recorded reference signal 2076 of the same heartbeat by a conventional ECG monitor. The detected signal 2075 is essentially without notable features and the entire heart related signal is masked by noise. Most prevalent in FIG. 24A is 60 Hz mains hum 2077, which is present in the reference signal as well. FIG. 24B illustrates the same two signals after filtering with a 30 Hz filter. The reference signal 2076 reveals an essentially intact and unobscured ECG signal. The detected signal reveals some periodic features, but with minimal amplitude or signal strength. FIG. 24C illustrates the modification of the detected signal 75 after amplification. Reference signal 2076 has not been modified. FIG. 24D illustrates only detected signal 2075 after additional signal processing and identification of the peaks 2077, as described more fully herein.

Another method for eliminating noise is that of filtering the signal in software residing either on either CPU 165 or on a separate computer to which the signal has been sent. In the preferred embodiment, this filtering consists of a non-linear filter designed to accentuate differences between noise and heartbeats. FIG. 24E shows the results of applying this filter. Detected signal 2075 is illustrated in box 2080 in an unfiltered state and in box 2079 after filtering.

While these noise reduction steps are likely to remove a significant amount of noise from the signal received from analog to digital converter 2160, it is likely that, notwithstanding this processing, there will still be noise remaining in the signal. This noise makes the task of identifying actual heart beat spikes from the signal for purposes of further processing, such as calculating a heart rate or other heart related parameters, difficult. Thus, a further aspect of the present invention relates to various processes and techniques, again implemented in software residing on either CPU 2165 or a separate computer, for identifying heart beat spikes from the signal notwithstanding any remaining noise. As will be appreciated, these processes and techniques, while preferably being performed after one or more of the noise reduction steps described above, may also be performed with any prior noise reduction steps having been performed.

As is well-known in the prior art, the Pan-Tompkins method uses a set of signal processing frequency filters to first pass only the signal that is likely to be generated by heart beats, then proceeds to differentiate, square and perform a moving window integration on the passed signal. The Pan-Tompkins method is described in Pan, J. & Tompkins, W.J., “A Real-time QRS Detection Algorithm,” IEEE Transactions on Biomedical Engineering, 32, 230-236 (1985), the disclosure of which is incorporated herein by reference.

According to this aspect of the invention, areas in the signal output by analog to digital converter 2160, with or without noise reduction as described above, having excessive noise, i.e., too much noise to practically detect acceptable heart beat spikes from the signal, are first identified and marked to be ignored in the processing. This may be done by, for example, identifying areas in the signal having more than a predetermined number of rail hits or areas in the signal within a predetermined time window, e.g., ¼ of a second, of two or more rail hits. Next, the remaining areas, i.e., those not eliminated due to too much noise being present, referred to herein as the non-noise signal, are processed to identify acceptable heart beat spikes for use in calculating various heart parameters such as heart rate.

In one embodiment of the present invention, acceptable heart beat spikes are identified in the non-noise signal by first identifying and then calculating the height and width of each peak in the non-noise signal as described above. Next, the width of each peak is compared to a predetermined acceptable range of widths, and if the width is determined to be within the acceptable range, the height of the peak is compared to an adaptive threshold height equal to 0.75 of the moving average of the height of the previous peaks. Preferably, the acceptable range of widths is 3 to 15 points when a 128 Hz analog to digital sampling rate is used, and represents a typical range of widths of a QRS portion of an ECG signal. Next, if the width of the current peak is within the acceptable range and if the height of the peak is greater than the adaptive threshold, then that peak is considered a candidate to be an acceptable peak for further processing. Peaks not meeting these requirements are ignored. Next, for candidate acceptable peaks within a predetermined timeframe of one another, preferably 3/16 of a second of one another, the heights of the peaks are compared to one another and the lower peaks in that time frame are ignored. If there is only one candidate acceptable peak within the timeframe, then that peak is considered a candidate acceptable peak. At this point, a number of candidate acceptable peaks will have been identified. Next, for each identified candidate acceptable peak, the area between that peak and the last, being that immediately previous in time, candidate acceptable peak is examined for any other signal peaks having a height that is greater than 0.75 of the height of the current candidate acceptable peak. If there are more than a predetermined number, preferably 2, such peaks identified, then the current candidate acceptable peak is invalidated and ignored for further processing. In addition, if there are any hits of the rail as described above between the last candidate acceptable peak and the current candidate acceptable peak, then the current candidate acceptable peak is invalidated and ignored for further processing. When these steps are completed, a number of acceptable peaks will have been identified in the signal, each one being deemed an acceptable heart beat spike that may be used to calculate heart related parameters therefrom, including, but not limited to, heart rate.

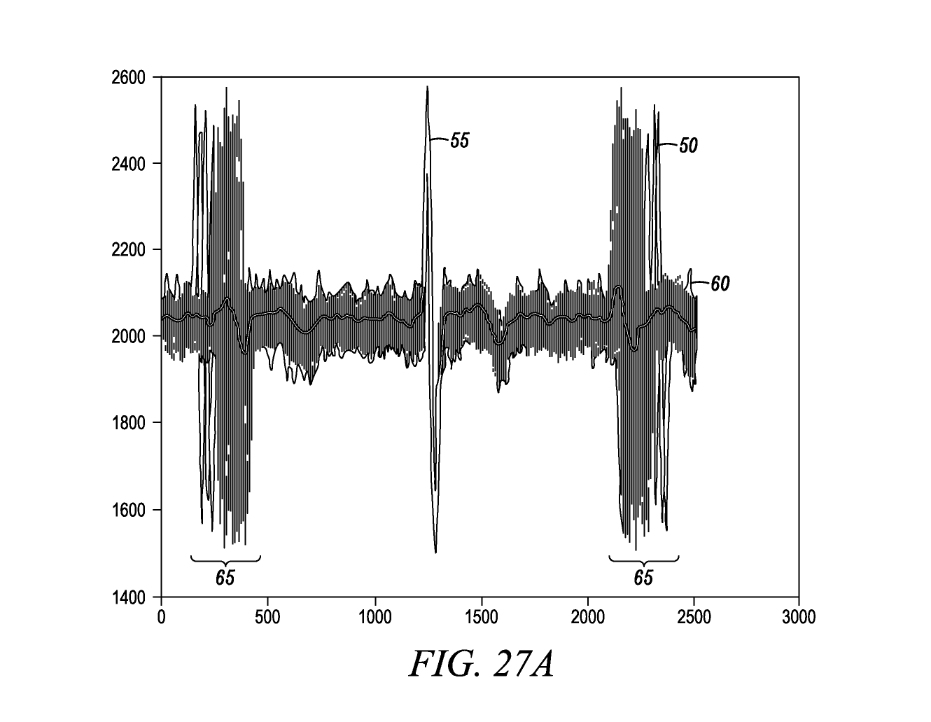

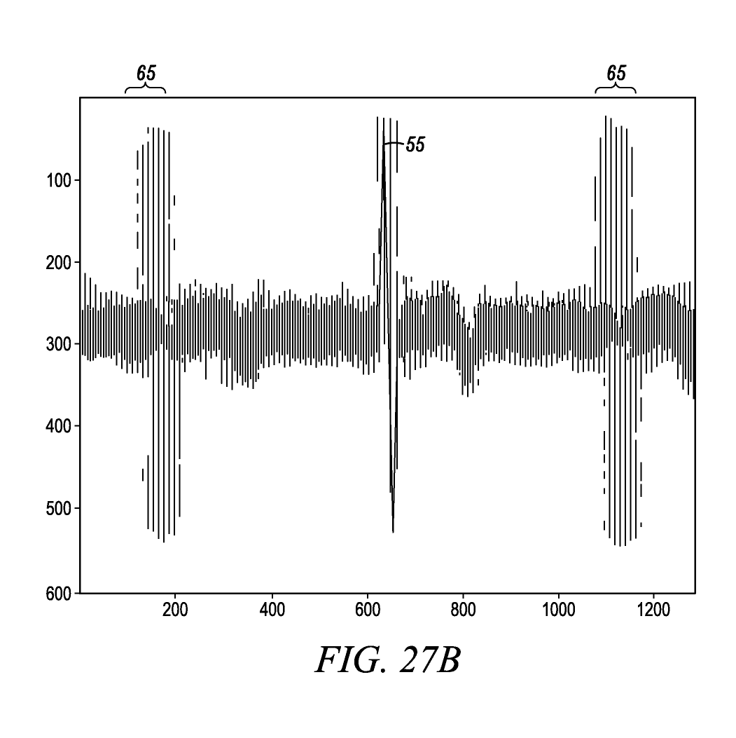

According to an alternate embodiment for identifying acceptable heart beat spikes, each up-down-up sequence, a possible QRST sequence, in the non-noise signal is first identified. As used herein, an up-down-up sequence refers to a sequence on the non-noise signal having an increasing amplitude portion followed by a maximum amplitude portion followed by a decreasing amplitude portion followed by a minimum amplitude portion followed by an increasing amplitude portion. An example of such up-down-up sequence is shown in FIG. 27 and includes points A, B, C, and D wherein the X axis is time and the Y axis is signal strength or amplitude. After each up-down-up sequence is identified, the height, in terms of amplitude, and the width, in terms of time, of each up-down-up sequence is calculated. Preferably, the height for each up-down-up sequence is determined as follows: (B_{Y}−A_{Y})+(B_{Y}−C_{Y})+(D_{Y}−C_{Y}), and the width for each peak is determined as follows: (D_{X}−A_{X}).

Next, the height of each up-down-up sequence is compared to a predetermined threshold value, preferably an adaptive threshold such as some percentage, e.g., 75%, of the moving average of previous heights, and the width of each up-down-up sequence is compared to a predetermined threshold value range, preferably equal to 4 to 20 points when a 128 Hz analog to digital sampling rate is used, which represents a typical range of widths of a QRST sequence of an ECG signal. If the height is greater than the threshold and the width is within than the predetermined threshold value range, then that up-down-up sequence is considered to be a candidate acceptable QRST sequence. Next, for each identified candidate acceptable QRST sequence in the non-noise signal, a surrounding time period window having a predetermined length, preferably 3/16 of a second, is examined and the height of the current candidate acceptable QRST sequence in the time period window is compared to all other identified candidate acceptable QRST sequences in the time period window. The candidate acceptable QRST sequence having the largest height in the time period window, which may or may not be the current candidate acceptable QRST sequence, is validated, and the other candidate acceptable QRST sequences in the time period window, which may include the current candidate acceptable QRST sequence, are invalidated and ignored for further processing. Once this step has been completed, a number of acceptable QRST sequences will have been identified in the non-noise signal. Next, for each acceptable QRST sequence that has been identified, the distance, in terms of time, to the immediately previous in time acceptable QRST sequence and the immediately next in time QRST sequence are measured. Each distance is preferably measured from the R point of one sequence to R point of the other sequence. The R point in each acceptable QRST sequence corresponds to the point B shown in FIG. 27, the highest amplitude point. In addition, two standard deviations are calculated for each acceptable QRST sequence. The first standard deviation is the standard deviation of the amplitude of all of the sampled points between the T point, which corresponds to point D shown in FIG. 27, of the current acceptable QRST sequence and the Q point, which corresponds to point A shown in FIG. 27, of the immediately next in time acceptable QRST sequence. The other standard deviation is the standard deviation of the amplitude of all of the sampled points between the Q point, which corresponds to point A shown in FIG. 27, of the current acceptable QRST sequence to the T point, which corresponds to point D shown in FIG. 27, of the immediately previous in time QRST sequence. Next, the two measured distances, the two standard deviations and the calculated height and width of each acceptable QRST sequence are input into a simple heart beat classifier, which decides whether the acceptable QRST sequence and the surrounding area is a qualifying heart beat or is too noisy. For example, the heart beat classifier may be a decision tree that has been trained using previously obtained and labeled heart beat data. Alternatively, the heart beat classifier may be any known classifier mechanism, including, but not limited to, decision trees, artificial neural networks, support vector machines, Bayesian belief networks, naïve Bayes and decision lists.

Those sequences that are determined to be too noisy are ignored. Thus, upon completion of this step, a set of acceptable QRST sequences will have been identified, the QRS, which corresponds to points A, B and C in FIG. 26, portion of each being deemed an acceptable heart beat spike that may be used to calculate various heart related parameters therefrom, including, but not limited to, heart rate.

According to an alternate embodiment for identifying acceptable heart beat spikes, each up-down-up sequence, a possible QRST sequence, in the filtered signal is first identified. The heights of the components of the sequence are then calculated. The allowed amplitude of the candidate QRST complexes are required to be at least double the estimated amplitude of signal noise. In addition, the width of the sequence must not exceed 200 milliseconds, an upper limit for believable QRST complexes. Next, if a candidate QRS complex is still viable, the plausibility of the location in time for the complex given the current heart rate estimate is checked. If the change in heart rate implied by the candidate beat is less than fifty percent then the sequence is identified to be a heart beat. FIG. 24F shows this process utilizing detected signal 2075, plotted as a series of interconnected data points forming QRST complexes in box 2081. Signal boundary boxes 2083 identify the two QRST complexes in detected signal 2075 which are eliminated because they fail the 50% test described above. Heart beat peak points 2084 are illustrated in box 2082 which represent the QRST complexes identified as beats from box 2081. Note the absence of heart beat peak points at the corresponding locations. Additionally, respiration data, including respiration rate, can be extracted from ECG waveforms. Respiration results in regular and detectable amplitude variations in the observed ECG. In terms of the equivalent dipole model of cardiac electrical activity, respiration induces an apparent modulation in the direction of the mean cardiac electrical axis.

Additional methodologies are presented for the analysis and display of the heart rate data. In each of these methods, the signal is serially segmented into a set of overlapping time slices based on identified QRST sequences. Each time slice is preferably exactly centered on the R point of a sequence and contains a fixed window of time, e.g. 1.5 seconds, on either side of the R point of that sequence. Each time slice may contain more than one QRST sequence, but will contain at least one in the center of the time slice. While the analysis is performed mathematically, a graphical description will provide the clearest understanding to those skilled in the art. Next, for a given point in time, some number of time slices before and after a given time slice are merged together or overlaid on the same graph. In one particular embodiment, 10 time slices before and after a given point are overlaid on the same graph In terms of graphic display, which is how this data may be presented to the user in the form of output, the time slice segments are overlapped, whereby some number of QRST sequences, or time slice segments, are overlaid on the same graph. Each detected primary QRST sequence and the neighboring sequences within the time slice segment, preferably 1.5 seconds, are overlaid on top of the other beats in that window. For example, in FIG. 27A, a series of signals 2050 are overlapped with each other with primary beat 2055 aligned between the overlapped signals. This is referred to as an AND-based overlapping-beat-graph. The average 2060 of all the superimposed beats is also calculated and displayed. At the center of the graph, where primary beats 2055 are aligned, the beats look very similar, and a clear signal is discernable. Also note that the neighboring beats 2065 are tightly clustered, with some deviation, which is an indicator of beat to beat variability. One skilled in the art will discern that the heart rate for this set of beats is easily extracted from such a graph by looking at the distance between the center QRS complex and the center of the neighboring complexes. When the signal is very clear, as in this example, the utility of this calculation is limited. However, when the signal is noisy and many false beats are detected, this technique can allow for finding a heart rate when the signal itself is too noisy to use simplistic or observational methods.

Another embodiment of the overlapping-beat-graph involves using a ADD-based approach to overlaps. In this version, as illustrated in FIG. 27B, when the beats and the neighboring signal overlap, the intensity of the pixel in the resulting graph is increased by the number of overlapping points. FIG. 27B illustrates an example for the ECG signal shown where the base color is black and each signal that overlaps makes the color brighter. Again, primary beat 2055 is utilized to align the time slice segments and the neighboring beats 2065 are shown as more of a cloud of points than in FIG. 27A. The width of this cloud of points is related to the beat to beat variability of the signal in question. Even though individual beats may not be reliably detected and the overlapped graph may not show a clear pattern in the lines, the average 2060, as shown in FIG. 27A may be utilized to identify clear neighboring QRS complexes. From these, a rate can be determined from the distance from the center of the time slice to the center of the cloud of points representing the neighboring QRS sequences. An ADD-graph may be utilized to identify distinct spikes for the neighboring QRS complexes in the presence of significant noise to enhance the capabilities of the system. In an alternate embodiment, the display could be biased more heavily toward those pixels with more overlapping points such that if the number of overlapping points is X at a particular pixel, its intensity could represented as X^{1.5}, thereby more selectively highlighting the most overlapped points.

A method of establishing a database or other reference for the morphologies of the user's heart beat signal would necessarily include the ability to classify heart beat patterns and to identify certain morphologies. These patterns and morphologies could then be associated with certain activities or conditions. The first step, however, is to identify the morphologies and patterns, as follows.

For example, a set of N ECG wave forms may be selected. The average distance between beats is identified and a time period ½ of the interbeat period before and ½ of the interbeat period after to truncate each waveform. It is specifically noted that other clipping distances are possible and could be variable. As with the descriptions of beat matching above, a graphic description of the process is the most illuminating. N signal wave forms are detected in the clipping mode and are modeled, as with the ADD graphs above, with the signal features being measured by the intensity or brightness. The signal is assigned an intensity or numerical value. The surrounding area has no value. The equator line of each wave form is identified, being that horizontal line such that the areas above and below this line are equal. A meridian line is identified for each wave peak as that vertical line that subdivides the QRS spike into two pieces, split at the peak value of the signal. All N images are overlapped such that all equators are coincident and all meridians are coincident. All intensity or numerical values for each point in the N signals are normalized such that all values are between two known boundary values, such as 0 and 1000. The result is a representation that captures the average heart beat morphology for that person over that period of time including, within the non-coincident areas, signal segments where the wave forms tend to be most coincident, having the highest values and the least coincident, having the lowest values. In addition, each of the N images could be scaled prior to overlap, wherein the height of the R point of each wave forms a constant. Additionally, accuracy may be increased by selecting X segments of X wave forms in row and performing the above analysis with the sequence of X wave forms instead of just with one.

As will be appreciated by those of skill in the art, it is possible that the signal output by analog to digital converter 2160 may have its polarity inverted as compared to what is expected from an ECG signal due to the placement of electrodes 2150, in which case what would otherwise be peaks in the signal will appear as valleys in the signal. In such a case, the processing described above may be successfully performed on the signal by first inverting its polarity. In one embodiment of the present invention, the signal output by analog to digital converter 2160 may be processed twice as described above, first without inverting its polarity and then again after its polarity has been inverted, with the best output being used for further processing as described herein. Additionally, the use of multiple sensors, such as an accelerometer or alternative pairs of electrodes, can be utilized to direct variable gain and dynamic signal thresholds or conditions during the signal processing in order to better adjust the types or nature of the processing to be applied. Additionally, a peak detector circuit may be employed such as that manufactured by Salutron, Fremont, Calif.

In addition, the system may detect known and recognizable contexts or signal patterns that will simply not present an acceptable signal that is discernable by the algorithms for beat and other body potential related feature detection. In these situations, the system simply recognizes this condition and records the data stream, such as when EMG or motion amplitude is at a peak level, the system detects this condition and discontinues attempting to process the signal until the next appropriate signal is received, according to certain preset or dynamically calculated conditions or thresholds. In some cases, the output of other sensors may be utilized to confirm the presence of a condition, such as excessive body motion, which would confirm that the system is operating properly, but lacking a coherent signal, as well as provide a basis for interpolation of the data from the missing segment of time. Under these conditions, a returned value from the system that no heart information could reliably collected is itself of value, relative to returning erroneous heart information.

Once acceptable heart beat spikes have been identified from the signal that is output by analog to digital converter 2160 using one of the methods described herein, the acceptable heart beat spikes may be used to calculate heart rate using any of several methods. While merely counting the number of acceptable heart beat spikes in a particular time period, such as a minute, might seem like an acceptable way to calculate heart rate, it will be appreciated that such a method will actually underestimate heart rate because of the fact that a number of beats will likely have been invalidated as noise as described above. Thus, heart rate and other heart related parameters such as beat to beat variability and respiration rate must be calculated in a manner that accounts for invalidated beats. According to one embodiment, heart rate may be calculated from identified acceptable heart beat spikes by determining the distance, in time, between each group of two successive acceptable heart beat spikes identified in the signal and dividing sixty seconds by this time to get a local heart rate for each group of two successive acceptable heart beat spikes. Then, an average, median and/or peak of all of such local heart rates may be calculated in a given time period and used as the calculated heart rate value.

In the event that a period of time is encountered where no signal is available of a minimum level of quality for beat detection, a methodology must be developed by which the events of this time period are estimated. The system provides the ability to produce accurate statements about some heart parameters, including heart rate, for this missing time period. A probability is assigned to the heart beat frequency based upon the prior data which is reliable, by taking advantage of previously learned data and probabilities about how heart rates change through time. This is not limited to the time period immediately prior to the missing time segment, although this may be the best indicator of the missing section. The comparison can also be made to prior segments of time which have been stored and or categorized, or through matching to a database of information relating to heart parameters under certain conditions. The system can also take advantage of other sensors utilized in conjunction with the device in these computations of probability. For example the probability of missing heart beats on the heart beat channels can be utilized given that the variance of the accelerometer sensor is high. This enables very accurate assessments of different rate sequences and allows the calculation of a likely heart rate. This method is most successful when some minimum number of detected beats are present.

An additional method of estimating activity during missing time periods is to first identify candidate beats using one of the methods discussed above. Any detection technique that also produces a strength value can be used. In the preferred embodiment the detector will associate a probability that the located beat is in fact a heart beat. Binary true/false detectors can be used by using as strength value 1 for truth. Next, all pairs of potential beats are combined to give a set of inter-beat gaps. Each inter-beat gap defines a weighting function whose values are based on a combination of the size of the gap, the amount of time which has passed since the gap was detected, the strength of the identification and any meta-parameters needed by the family of weighting functions. In the preferred embodiment this weighting function is the inverse notch function. The inter-beat gap, in units of seconds, determines the location of the notch's peak. The height of the notch is driven by the strength of the identification, the length of time since the gap was identified, as age, and a hyper-parameter referred to as lifetime. The width of the notch is defined by the hyper-parameter width. FIG. 24G shows this inverse notch function including notch peak 2087 and notch width 89. The function itself is mathematically expressed as:

w(X,gap,strength,age,lifetimewidth)=max(0,(1−age/lifetime)*strength*1−abs(X−gap)/width))

In the third step, the individual weighting functions are summed to obtain a total weighting function. Finally, the resulting function is programmatically analyzed to obtain an estimate of heart rate.

In the preferred embodiment, the estimate of the true inter-beat gap is taken to be the value at which the function reaches its first local maximum. FIG. 24H shows the resulting function and indicates the first local maximum 2091. Once the inter-beat gap is selected, the heart rate is determined from the formula HeartRate=60/InterbeatGap.

To minimize the processing load associated with the evaluation of the total weighting function, those individual weighting functions whose inter-beat gaps are either larger or smaller than is physiologically possible are eliminated. In addition, individual functions whose age has exceeded the value of the lifetime hyper parameter are also eliminated.

Another embodiment utilizes probabilistic filters on the allowed inter-beat gaps instead of a hard truncation as described above. These probabilistic filters take as input one or more signals in addition to the ECG signal and determine a probabilistic range for the allowable heart beat. One instantiation of this is to determine the context of the wearer from the non-ECG signals and then, for each context, to apply a particular Gaussian distribution with parameters determined by the context, the wearer's body parameters, as well as the ECG signal itself. Other probability distributions can easily be utilized as well for this biasing. This probability can then be multiplied by the probability of each inter-beat gap to produce a posterior distribution, from which the most likely heart beat can be easily determined.

Another aspect of the present invention is that during times when certain heart parameters are not computable due to noise, these parameters can also be estimated from the set of measured values nearby in time and the sequences of other measurements made on other sensors. One such embodiment of this method is a contextual predictor similar to that used for energy expenditure, but instead used to predict heart rate from accelerometer data, galvanic skin response data, skin temperature and cover temperature data, as well as steps taken and other derived physiological and contextual parameters. This method first identifies the wearer's activity, and then applies an appropriate derivation for that activity. In the preferred embodiment, all derivations for all activities are applied and combined according to the probability of that activity being performed.

An additional aspect of the invention is a method of adaptation over time for a particular user through the use of multiple noisy signals that provide feedback as to the quality of other derived signals. Another way of viewing this is as a method of calibration for a given user. First, a given derived parameter is calculated, representing some physiological state of the wearer. Second, a second derived parameter is calculated, representing the same physiological state. These two derived parameters are compared, and used to adjust one another, according to the confidences calculated for each of the derived metrics. The calculations are designed to accept a feedback signal to allow for training or tuning them. In one embodiment, this consists of merely utilizing gradient descent to tune the parameters based on the admittedly noisy feedback signal. In another embodiment, this involves updating a set of constants utilized in the computation based on a system of probabilistic inference.

According to one aspect of the present invention, an algorithm development process, as described in detail above, is used to create a wide range of algorithms for generating continuous information relating to a variety of variables from the data received from the plurality of physiological and/or contextual sensors on armband body monitoring device 300, as identified in Table I hereto, including the ECG signal generated using electrodes 2105 that is used to calculate heart rate and other heart related parameters, many of which cannot be distinguished by visual recognition from graphical data output and diagnostics alone. These include heart rate variability, heart rate deviation, average heart rate, respiration rate, atrial fibrillation, arrhythmia, inter-beat intervals, inter-beat interval variability and the like. Additionally, continuous monitoring of this type, coupled with the ability to event- or time-stamp the data in real time, provides the ability to titrate the application of drugs or other therapies and observe the immediate and long term effects thereof. Moreover, the ability is presented, through pattern recognition and analysis of the data output, to predict certain conditions, such as cardiac arrhythmias, based upon prior events. Such variables may include, without limitation, energy expenditure, including resting, active and total values; daily caloric intake; sleep states, including in bed, sleep onset, sleep interruptions, wake, and out of bed; and activity states, including exercising, sitting, traveling in a motor vehicle, and lying down. The algorithms for generating values for such variables may be based on data from, for example, an axis or both axes of a 2-axis accelerometer, a heat flux sensor, a GSR sensor, a skin temperature sensor, a near-body ambient temperature sensor, and a heart rate sensor in the embodiments described herein. Additionally, through the pattern detection and prediction capabilities described above, the system may predict the onset of certain events such as syncope, arrhythmia and certain physiological mental health states by establishing a known condition set of parameters during one such episode of such an event and detecting similar pre-event parameters. An alarm or other feedback would be presented to the user upon the reoccurrence of that particular set of parameters matching the prior event.

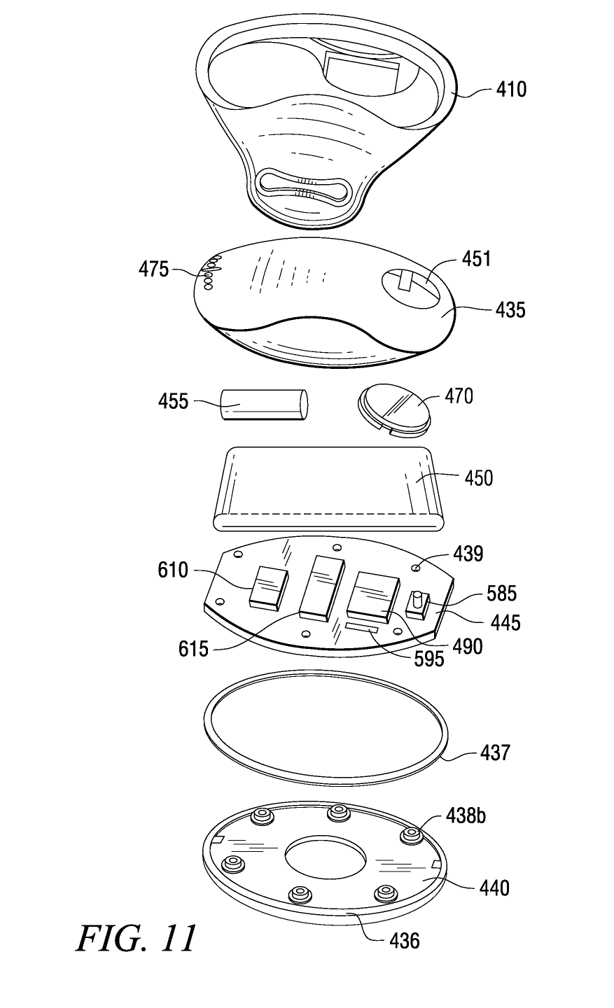

As another example, an algorithm having the format shown conceptually in FIG. 11 may be developed for measuring energy expenditure of an individual that utilizes as inputs the channels derived from the sensor data collected by armband body monitoring device 300 from the 2-axis accelerometer and the electrodes 105, from which heart rate and/or other heart related parameters are calculated. The parameters derived from these motion and heart rate sensor types are largely orthogonal and are very descriptive of a user's activities. The combination of these two sensors in an algorithm having the format shown conceptually in FIG. 14 provides the ability to easily distinguish between different activity classes that might be confusing to a single sensor, such as stressful events, some of which could be identified by high heart rate and low motion, vehicular motion events, some of which could be identified by low heart rate and high motion and exercise events, some of which could be identified by high heart rate and high motion. As shown in FIG. 11, in this embodiment, the channels derived from the sensor data from these two sensors are first used to detect the context of the user. The appropriate function or functions are then used to predict energy expenditure based on both heart rate and motion data. As a further alternative, channels derived from additional sensors forming a part of armband body monitoring device 300, such as a heat flux sensor may also be used as additional inputs into the algorithm. Using heart rate in an algorithm for predicting energy expenditure can result in a better, more accurate prediction for a number of reasons. For example, some low motion exercises such as biking or weight lifting pose issues for an energy expenditure algorithm that uses arm motion from an accelerometer as a sole input. Also, clothing may adversely affect measurements made by a heat flux sensor, which in turn may adversely effect energy expenditure predictions. Incorporating heart rate or other heart related parameters into an algorithm helps to alleviate such problems. Obviously, there is considerable utility in the mere detection, analysis and reporting of the heart rate and other heart related parameters alone, other than for use in such algorithms. Moreover, heart rate generally slows when someone falls asleep, and rises during REM periods. Thus, algorithms for predicting whether someone is sleeping and what stage of sleep they are in may be developed in accordance with the present invention that utilize as an input, along with other sensor data, data collected by armband body monitoring device 300 from the electrodes 2105 from which heart rate and/or other heart related parameters are calculated as well as the other detected data types identified herein. Such heart related data may also be used in algorithms for detecting various sleep disorders, such as sleep apnea. Similarly, when under stress, a person's heart rate often rises without an accompanying increase in motion or body heat. Day to day or time period to time period comparisons of such data for an individual will assist in identifying certain patterns or conditions which may be used for both further pattern detection or prediction. Algorithms for detecting stress may be developed in accordance with the present invention that utilize data collected from the electrodes 2105, from which heart rate and/or other heart related parameters are calculated, along with other sensor data such as data from an accelerometer. While the applicability of recognizing stress is most likely in the context of reviewing past activity and attempting to correlate the detected and derived parameters with life activities or other non-detectable events, the ability to detect stress may be effective as a contemporaneous measurement to identify a condition that may be masked from the wearer by external conditions or merely preoccupation. This is especially true in the event that the heart is undergoing stress in the absence of physical exertion or activity.

Other important feedback embodiments include the ability to detect REM sleep through the heart related parameters and to maximize the wearer's opportunity to engage in such sleep. Rather than the conventional alarm waking the user at a preappointed time, the alarm could wake the wearer after a preset amount of REM sleep, and further at an appropriate endpoint of such sleep or during or just after some particular sleep stage.

In the most preferred embodiment, armband body monitoring device 300 includes and/or is in communication with a body motion sensor such as an accelerometer adapted to generate data indicative of motion, a skin conductance sensor such as a GSR sensor adapted to generate data indicative of the resistance of the individual's skin to electrical current, a heat flux sensor adapted to generate data indicative of heat flow off the body, a electrodes for generating an ECG signal from which data indicative of the rate or other characteristics of the heart beats of the individual may be generated, and a temperature sensor adapted to generate data indicative of a temperature of the individual's skin. In this preferred embodiment, these signals, in addition the demographic information about the wearer, make up the vector of signals from which the raw and derived channels X are derived. Most preferably, this vector of signals includes data indicative of motion, resistance of the individual's skin to electrical current, heat flow off the body, and heart rate.

Another specific instantiation where the present invention can be utilized relates to detecting when a person is fatigued. Such detection can either be performed in at least two ways. A first way involves accurately measuring parameters such as their caloric intake, hydration levels, sleep, stress, and energy expenditure levels using a sensor device and using the two function (f₁ and f₂) approach to provide an estimate of fatigue. A second way involves directly attempting to model fatigue using the direct derivational approach described in connection with FIGS. 14 and 15. The first way illustrates that complex algorithms that predict the wearer's physiologic state can themselves be used as inputs to other more complex algorithms. One potential application for such an embodiment of the present invention would be for first-responders, e.g. firefighters, police, soldiers, where the wearer is subject to extreme conditions and performance matters significantly. For example, if heat flux is too low for too long a period of time but skin temperature continues to rise, the wearer is likely to experience severe heat distress. Additionally, the ability to detect the wearer's hydration level and the impact of the deterioration of that level is quite useful, and may be derived utilizing the multiple sensors and parameters detected by the system. When a person becomes dehydrated, they typically experience an initially high level of perspiration, which then drops off. The body loses its ability to cool, and heat flux changes are detected. Additionally, the body temperature rises. At this point the cardiovascular system becomes less efficient at transporting oxygen and heart rate increases to compensate, possibly as much as 10-20%, necessitating an increase in respiration. At later stages, the user experiences peripheral vascular shutdown which reduces blood pressure and results in degradation in activity, awareness and performance. The monitoring system, which would be capable of measuring and tracking the hydration level, works in conjunction with the ECG detection, which, by measuring the relative changes in amplitude over time, in conjunction with expended energy, will recognize and confirm that amplitude changes are unexpected, or expected because of the events to current time.

It will be appreciated that algorithms can use both calibrated sensor values and complex derived algorithms. This is effective in predicting endpoints to or thresholds of certain physiological conditions and informing the wearer or other observer of an approximate measure of time or other activity until the endpoint is likely to be reached.

Another application of the current invention is as a component in an apparatus for doing wearer fingerprinting and authentication. A 128-Hz heart-rate signal is a rich signal, and personal characteristics such as resting heart rate, beat to beat variability, response to stimuli, and fitness will show up in the signal. These identifying personal characteristics can be used to verify that the wearer is indeed the approved wearer for the device or to identify which of a range of possible approved wearers is currently wearing the device. In one embodiment of this aspect of the invention, only the 128-hz signal and derived parameters from that signal are utilized for identification. In another, all of the sensors in the monitor are used together as inputs for the identification algorithm.

In another application of this aspect of the invention, an authentication armband can be utilized in a military or first responder system as a component in a friend or foe recognition system.

Interaction with other devices is also contemplated. The system can augment the senses and also the intelligence of other products and computer systems. This allows the associated devices to collectively know more about their user and be able to react appropriately, such as automatically turning the thermostat in the house up or down when asleep or turning the lights on when awakened. In the entertainment context, the detection of certain stress and heart related parameters may be utilized to affect sound, light and other effects in a game, movie or other type of interactive entertainment. Additionally, the user's condition may be utilized to alter musical programming, such as to increase the tempo of the music coincident with the changing heart rate of the user during exercise or meditation. Further examples include turning the car radio down when the person gets stressed while they drive because they're looking for an address; causing an appliance to prepare a caffeinated drink when the person is tired; matching up people in a social environment in the same mood or with the same tastes; utilizing alertness and stress indicators to tune teaching systems such as intelligent tutors or flight simulators, to maximize the student's progress; removing a person's privileges or giving a person privileges based on their body state, for example not letting a trucker start up his truck again until he has had 8 hours of sleep; providing automatic login to systems such as the wearer's personal computer based on biometric fingerprinting; and creating new user interfaces guided in part or in whole by gross body states for impaired individuals such as autistic children.

Moreover, new human-computer interactions can be envisioned that use bio-states to adjust how the computer reacts to the person. For example, a person is tele-operating a robotic arm. The system can see he is tired and so smoothes out some of his motion to adjust for some expected jerkiness due to his fatigue.

Individuals with suspected heart rhythm irregularities will often undergo some type of home or ambulatory ECG monitoring. Quite often, the individual's symptoms appear infrequently and irregularly, such as one a day, once a week, once a month, or even less often. In such cases, it is unlikely that the symptoms will be detected during a visit to the doctor in which classic ECG measurements are taken. Thus the need for home or ambulatory ECG monitoring to attempt to capture such infrequent episodes. The most common home or ambulatory ECG monitoring methods are Holter monitoring, event recording, and continuous loop recording, as described above.

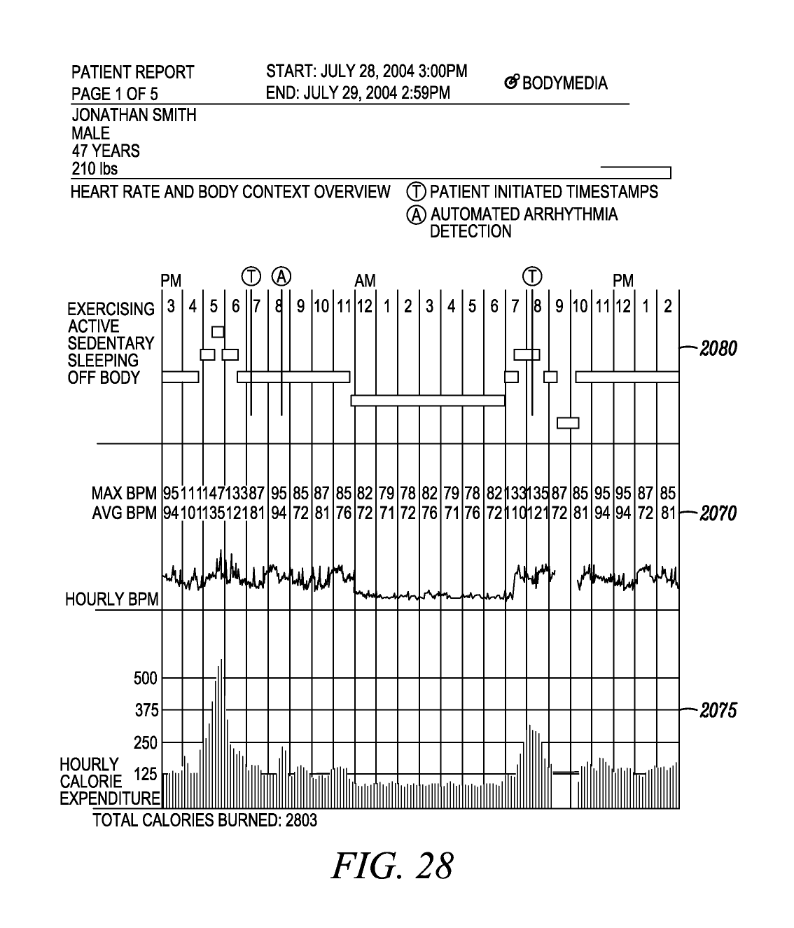

According to another aspect of the present invention, a device as described herein that measures an ECG signal may be adapted and configured to perform the functionality of a Holter monitor, an event recorder, or a continuous loop recorder. Preferably, such a device may be armband body monitoring device 300 as illustrated and described herein. Such a device may be comfortably worn for extended periods of time, unlike a Holter monitor or an event recorder on a convenient location on a limb, such as the upper arm in the case of armband body monitoring device 300. In addition, the recorded ECG signals may be combined with other data that is contemporaneously measured by such a device in accordance with other aspects of the present invention described herein, including the various physiological parameters and/or contexts that may be predicted and measured using the algorithms described herein, to provide automatically context and/or parameter annotated heart related information. For example, as shown in FIG. 28A, a measured ECG signal 2070 for a period of time may be mapped or presented along with measured parameters such as energy expenditure 2075 or even raw sensor values and detected contexts 2080 such as walking, driving and resting for the same period of time. This annotated view of the ECG signal would be useful to a health care provider because it will identify what the individual was doing while certain heart symptoms were occurring and will provide certain other physiological parameters that may assist with diagnosis and treatment. This may be accomplished, for example, by downloading the measured ECG signal, the measured parameter or parameters and the detected contexts to a computing device such as a PC which in turn creates an appropriate display.

It is also well known that there is a circadian pattern to certain arrhythmias or conditions which lead to heart related stress. Sudden cardiac arrest, for example, has a high incidence in early morning. It is therefore anticipated that the detection might be enhanced during certain time periods, or that other devices could be cued by the monitoring system to avoid certain coincident or inappropriate activities or interactions. A pacemaker, for example could raise pace according to a preset protocol as the wearer comes out of sleep or waking the user calmly at the end of a REM stage of sleep.

The system is further applicable in diagnostic settings, such as the calibration of drug therapies, post-surgical or rehabilitative environments or drug delivery monitoring, with immediate and real time effects of these medical applications and procedures being monitored continuously and non-invasively.

This type of application may also be utilized in a mass emergency or other crisis situation, with victims being collected in one location (for example a gymnasium) and are being seen by nurses, EMTs, physicians, volunteers—where this staff is basically short staffed for this type of situation and diagnosing or keeping watchful monitoring over all the victims now patients (some quite injured and others under observation in case the injury or shock are delayed in terms of physical/tactile/visual symptoms). A system having diagnostic heart related capabilities and, optionally, hydration, hypothermia, stress or shock could be distributed upon each victim's entrance for monitoring. The design of the system, which alleviates the need to remove most clothing for monitoring, would both speed and ease the ability of the caregivers to apply the devices. This system could send the alerts to a central system in the facility where the serial number is highlighted, and the attendant is alerted that a condition has been triggered, the nature of the condition as well as the priority. Within this collaborative armband scenario, all the armbands around the condition sensing/triggering armband could also beep or signal differently to focus the attention of an attendant to that direction more easily. Additionally, certain techniques, as described below, would allow all of the armbands to interactively coordinate and validate their relative location continuously with the surrounding armbands, allowing the central monitoring station to locate where in the facility the location of any particular armband is located and where specifically are the individuals who need the most immediate attention.

More specifically, the device could be designed to be part of a network of devices solving as a network of devices the exact or relative locations of each device in the network. In this embodiment each device would have one or more mechanisms for determining the relative position of itself to another device in the network. Examples of how this could be done include the sending of RF, IR, or acoustic signals between the devices and using some technique such as time of flight and/or phase shifts to determine the distance between the devices. It is a known problem that methods such as these are prone to errors under real world circumstances and in some cases, such as the phase shift method, give the receiving device an infinite number of periodic solutions to the relative distance question. It is also typical that such devices, because of power limitations, occasional interference from the environment and the like, would lose and then later regain contact with other devices in the networks so that at any one time each device might only have communication with a subset of the other devices in the network.

Given this ability to establish at each moment in time a relative distance between each pair of devices, and the ability of the devices to share what they know with all other devices in the network, for a network for N devices, there are a total of (N*(N−1))/2 distances to be measured and it is practical that every device could, by passing on all they know to all the devices they can communicate with at that moment in time, arrive at a state where all devices in the network have all available relative distances that could be measured, which would be some subset of the (N*(N−1))/2 possible distances to be measured, and could have updates to this list of numbers quite often, e.g. several times per minute, relative to the speed at which the wearers are changing relative to each other.

Once each device has a list of these distances, each device effectively has a system of equations and unknowns. For example: A is approximately X meters from B, B is approximately Y meters from C, C is approximately Z meters from A, A is U meters from D, B is T meters from D, C is V meters from D. Alternatively, under the phase shift only model, these equations could be as follows: A is some integer multiple of six inches from B, B is some integer multiple of eight inches from C, C is some integer multiple of one foot from D, and D is some integer multiple of seven inches from A. To the extent there is redundant information in the network, as in the examples just given, and with the possible additional assumptions about the topology on which the wearers are situated, such as a flat area, a hill that rises/falls no faster than a grade of 6% or the like, each device can solve this system of equations and unknowns or equations and inaccurate values to significantly refine the estimates of the distance between each pair of devices. These results can be then shared between devices so that all devices have the most accurate, up-to-date information and all agree, at each moment in time, what their relative positions are. This solving of equations can be done through a process such as dynamic programming or a matrix solution form such as singular value decomposition. The previous values each wearer's device has for its distance to all the other devices can be included in these calculations as follows to take advantage for things such as if A was ten feet from B five seconds ago, it is highly unlikely that A is now two hundred feet from B even if that is one of the possible solutions to the system of equations and unknowns.

An alternative embodiment involves utilizing probabilistic reasoning to keep track of a probabilistic estimate of the relative location of each wearer and for taking into account possible sensor noise and expected motion. Kalman filters are an example of this sort of reasoning often applied in tracking a single moving entity; extensions to multiple interacting entities are available.

If these devices are also equipped with ability to know or be told, from time to time, their actual or approximate global location, such as through an embedded GPS chip, then this information could also be shared with all the other devices in the network so that, adjusting for their relative distances, each device will then know its global location.

To aid in this process, it is preferred that there be provided at least one interval where the relative positions are known for the entire network. This, along with frequent updates, relative to the rate they move relative to each other, to the relative distances of the devices, reduces the possibly solutions for these systems of equations and thereby improves the accuracy of the process. This synchronization of the devices could be accomplished for example, for having them together in the identical location for a moment before each devices sets out on its own for a time.

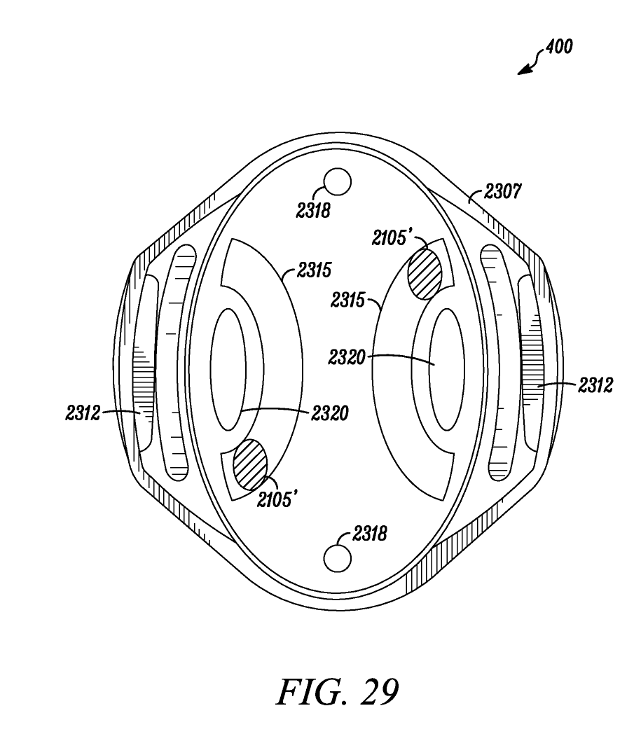

Referring now to FIGS. 29 and 30, armband body monitoring device 300 is provided with additional physiological and/or contextual sensors for sensing various physiological and/or contextual parameters of the wearer, including, but not limited to, GSR sensors 2315 for measuring the resistance of the skin to electrical current, a heat flux sensor in thermal communication with heat flux skin interface component 320 for measuring heat flow off of the body, a skin temperature sensor in thermal communication with skin temperature skin interface component 325 for measuring skin temperature, a body motion sensor such as an accelerometer (not shown) for measuring data relating to body movement, and an ambient temperature sensor (not shown) for measuring the near-body temperature of the wearer. Referring to FIG. 29, at least one, and preferably two electrode support connectors 318 are provided for the temporary and removable attachment of any one of a series of electrode support modules. Referring to FIG. 30, circuit 2200 including electrodes 2105A through 105D may be provided as part of an armband body monitoring device 2300 such as are described in the aforementioned U.S. Pat. No. 6,605,038 and U.S. application Ser. No. 10/682,293, owned by the assignee of the present invention (see, e.g., sensor devices 400, 800 and 1201 described in the '038 patent and/or the '293 application), connected to housing 2305 and circuit 2200 through insulated wires 2310. Electrodes 2105′ are illustrated in FIGS. 29, 30 and 33 at alternative locations at various locations on the housing or support members. It is to be specifically noted that electrodes may be placed at any appropriate location on or associated with the housing for the purpose of engaging the corresponding appropriate locations on the body for detecting a signal of appropriate strength and aspect. With respect to FIG. 29, the alternative electrodes 2105′ are located within GSR sensors 2315. With respect to FIG. 30, alternative electrodes 2105′ are mounted directly within housing 2305.

Armband body monitoring device 2300 is designed to be worn on the back of the upper arm, in particular on the triceps muscle of the upper arm, most preferably the left arm. Referring to the specific embodiment shown in FIG. 30, when worn on the upper left arm, electrode 2105A is in contact with the deltoid muscle, electrode 2105B is in contact with the triceps muscle, electrode 2105C and electrode 2105D are in contact with an area of the muscle which may not produce a detectable heart related signal but permits the detection of baseline EMG noise. Preferably, first and second imaginary diagonal lines connect electrode 2105A to electrode 2105 B and electrode 2105C to electrode 2105D, respectively, at angles of approximately 31 degrees from vertical. In this embodiment, electrodes 2105A and 2105B may be paired with one another to detect a first signal and electrodes 2105C and 2105D may be paired with one another to detect a second signal as described above, which signals are summed together by summation circuit 2170 of circuit 2200.

Referring now to FIG. 31, an alternative embodiment of the device illustrated in FIG. 30 is shown. Electrode support connector 2318 is provided for the purpose of physically supporting a sensor or sensor support housing as well as establishing electrical communication therewith. Electrode support connector 2318 may be a plug-in or snap-in connector of the pin type which will provide good physical support while allowing some degree of movement or rotation of the sensor or sensor housing while mounted on the body. Preferably, the device and sensor or sensor support, as appropriate, are integrated for best physical and electrical connection. A multichannel electrical connection is also provided according to conventional means, typically utilizing multiple independently insulated segments of the supporting connector. A sensor support housing 2322 may be provided for the support and positioning of electrode 2105, as shown in FIG. 31, or the electrode 2105 or other sensor may be directly and independently mounted to electrode support connector 2318. In this embodiment, the support housing 2322, is entirely substituted by the electrode 2105 itself in an identical physical arrangement. The electrode 2105 may be positioned at any point on the surface of support housing 2322, and need not be located at the center, as shown in FIG. 31. Additionally, sensors need not be a point source of information, as they are conventionally applied and utilized. The sensor may further be comprised of a broad segment of sensitive material which covers a substantial portion of the housing surface in order to maximize the location of the appropriate point for signal detection within the surface area of the sensor. In the event that a support housing 322 is utilized, a flexible material is utilized to permit the housing to conform to the surface of the arm upon which it is mounted to ensure good contact with the skin and underlying tissue. This is equally applicable to the embodiment shown in FIG. 30. It is also to be specifically noted that each of the sensor, electrode and support housing embodiments described and illustrated herein are interchangeable, with certain shapes or other physical parameters being selected for particular applications. Additionally, it is to be understood that the number and arrangement of the sensors, electrodes and support housings are not limited by the embodiments shown in the Figures, but may be interchanged as well. Lastly, in order to establish a particular geometry of sensors, electrodes or an array of the same, the housing 305 of the device may be modified to be elongated or diminished in any particular dimension for the purpose of improving the signal, as described above.

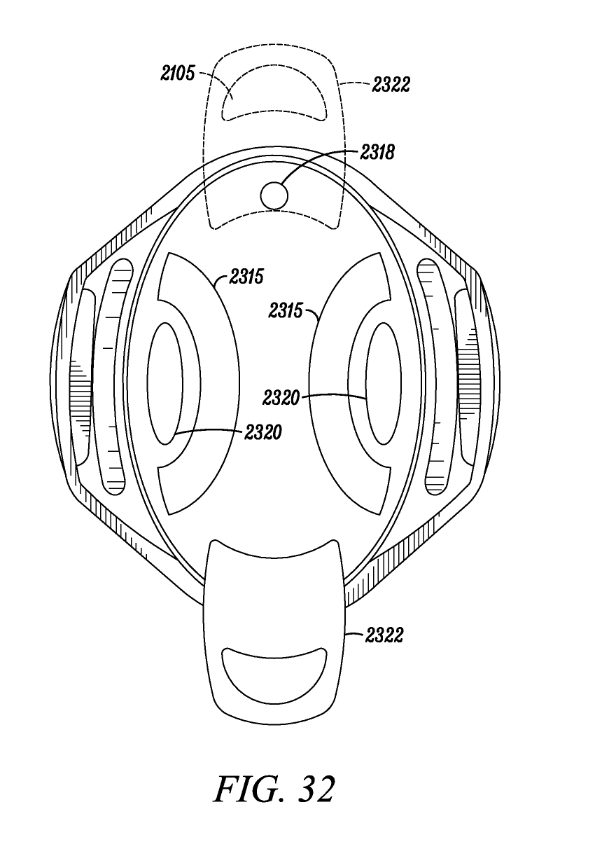

With reference to FIG. 32, an additional alternative embodiment is illustrated which provides a similar orientation of electrodes as that illustrated in FIG. 31, with the support housing 2322 having a more elongated geometry. Typically, more elongated or outboard electrode placements will necessitate the use of more firm materials for the support housing 2322, in order to maintain good skin contact. It is to be specifically noted that any of the housing embodiments shown and illustrated may further comprise a flexible or partly flexible housing section which is pre-molded in a curved embodiment in order to exert pressure against the skin.

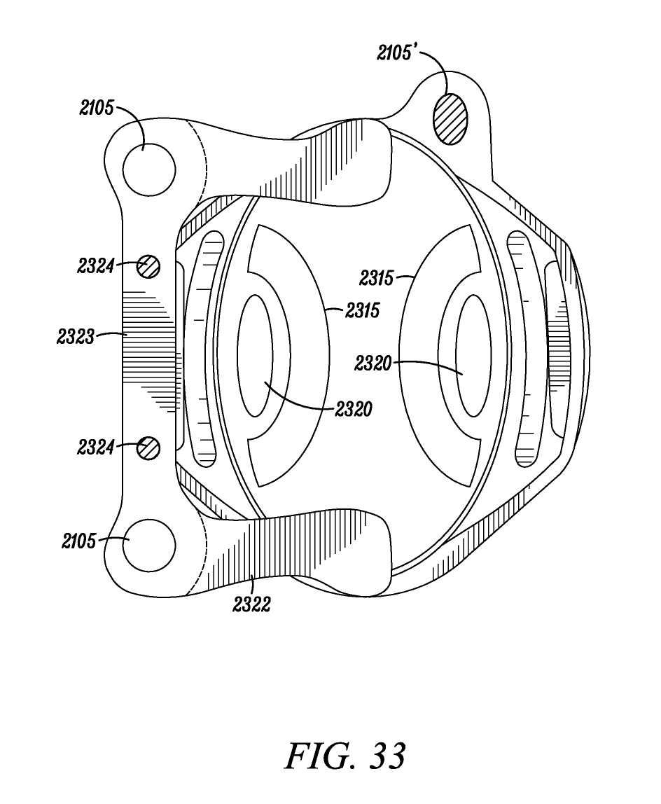

FIG. 33 illustrates an asymmetrical arrangement of the support housing 2322 having a lateral support arm 2323 which is intended to specifically place the upper and lower electrodes 2105 adjacent to the deltoid and brachialis sections of the tricep muscle, respectively, of the human upper arm. Lateral support arm 3223 may also be separated from support housing 2322 along the chain line sections indicated in the figure and affixed to wings 2311 by restraints 2324. Housing 2305 or wings 2311 may further be extended beyond the generally ovoid shape illustrated in the figures hereto into any particular shape necessary to engage the appropriate locations on the body. More particularly, irregular extensions of housing 2305 or wings 2311 are contemplated to mount alternative electrodes 2105′.

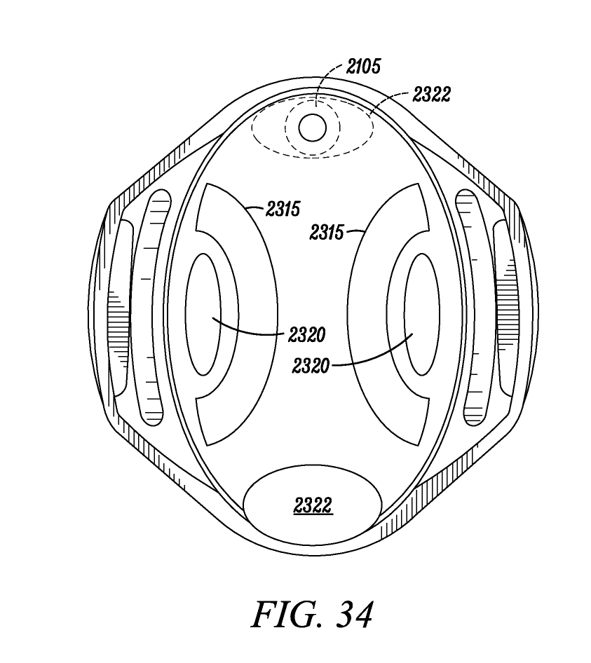

FIG. 34 illustrates support housing 2322 having a particular ovoid shape.

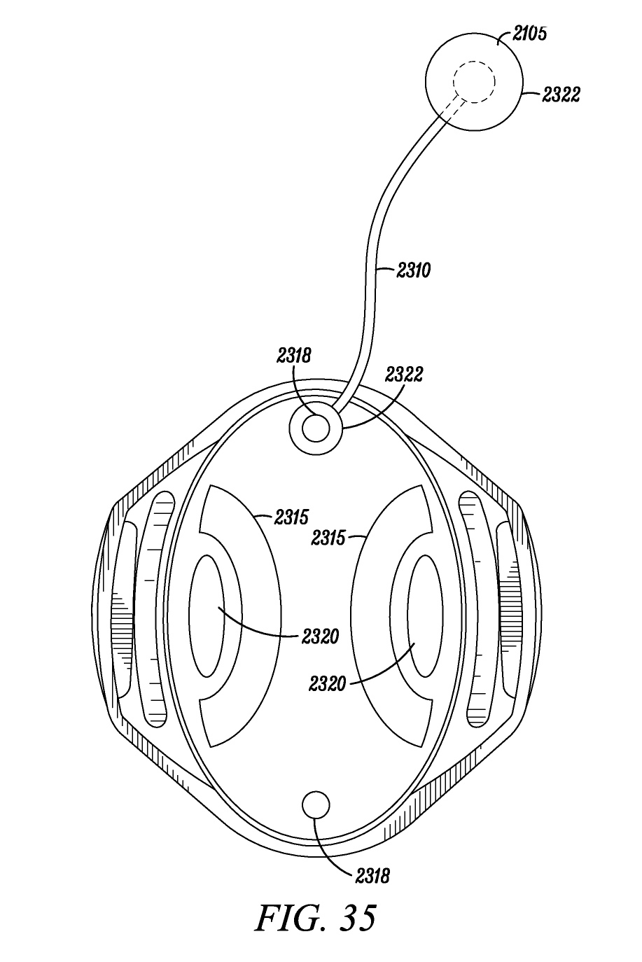

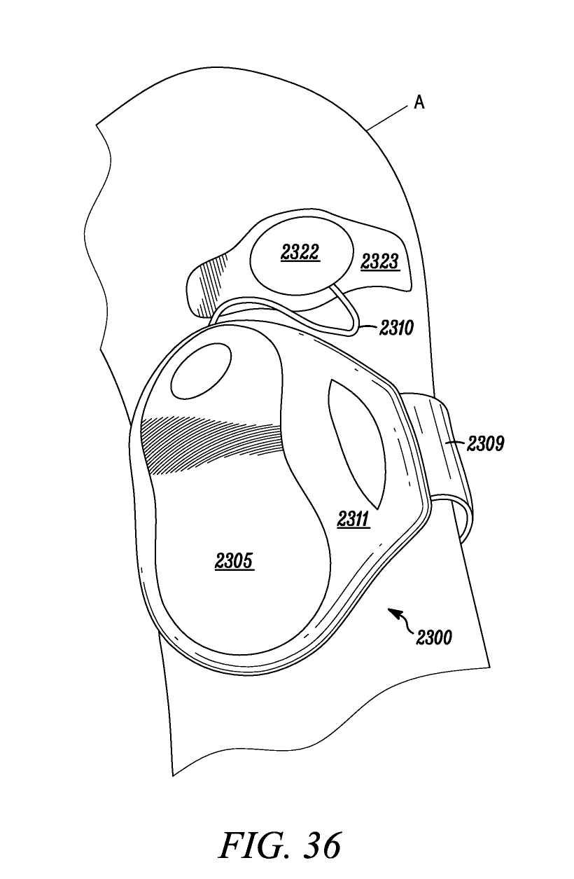

FIG. 35 illustrates an alternative embodiment similar to that illustrated in FIG. 30, however only one outboard or external electrode 2105 is utilized, which is provided with electrical communication through insulated wire 2310. Any of the previously identified electrode geometries may be utilized for affixation to the second electrode support connector 2318. The use of the outboard electrode 2105 connected to insulated wire 2310, sometimes identified as a fly lead, is adapted for particular location on a remote section of the body which renders the creation of an integrated housing 2305 of armband body monitoring device 2300 impractical. FIG. 36 illustrates the embodiment of FIG. 30 mounted upon a human upper arm A. Armband body monitoring device 2300 is placed adjacent the skin at an appropriate position and the elastic strap 2309 encircles the arm and is pulled tight enough to firmly secure the housing without reducing blood flow. Sensor support housing 2322 supports electrode 2105 (not shown) and is held in place by adhesive support 2323 which mounts support housing 2322 to the skin. It is to be specifically noted that the location of the support housing is not limited to the location illustrated in FIG. 36, but may extend to any part of the body, including the other arm of the wearer. The most preferred embodiment seeks to minimize the use and length of insulated wires 2310.

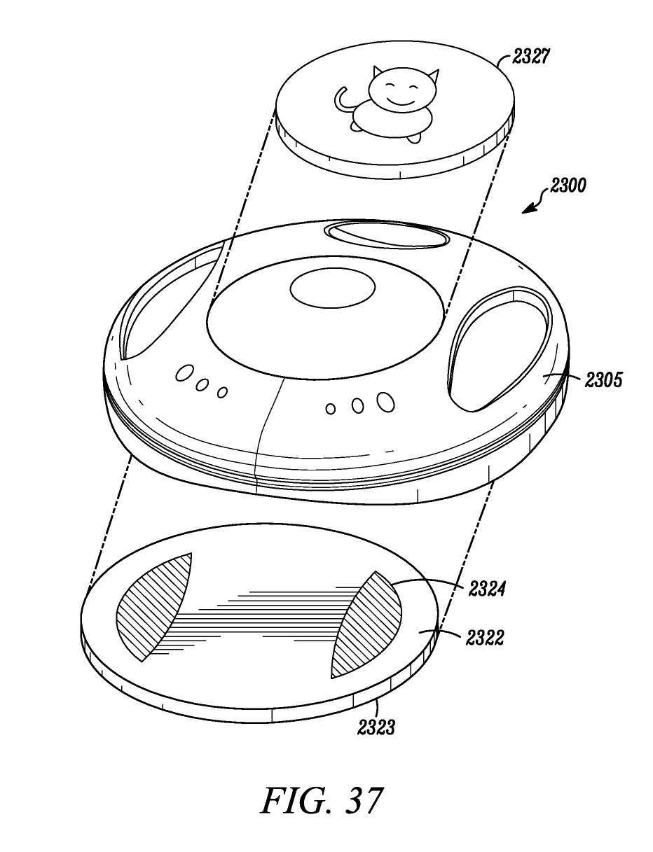

FIG. 37 illustrates an alternative embodiment which presents a more modular approach to the interface between the electrodes 2105, support housing 2322 and housing 2305. Housing 2305 is provided with a similar skin engagement face (not shown) as illustrated in FIG. 29. An integrated removable support housing 2322, which may be disposable, comprises both the support material for exerting the appropriate force upon the electrodes (not shown) on the underside of the support housing 2322 against the skin, the electrodes themselves, as well as the electronic connections between the electrodes and the housing 2305. Support housing is provided with at least one electrode contact 2324 for electronic engagement with the housing, and may be suited for engagement with either electrode support connectors 2318 or GSR sensors 2315 which have been specifically adapted to communicate with electrodes 2105 in conjunction with support housing 2324. An optional adhesive support 2323 may also be provided on the underside of support housing 2322. In an alternative embodiment, adhesive support 2323 may provide the sole means for retention of housing 2305 on the user's arm. Support housing 2322 may also be supported on the skin solely by the force of the housing 305 as restrained on the arm by elastic strap 2309, or in conjunction with other housing or garment support devices as described in U.S. patent application Ser. No. 10/227,575, the specification of which is incorporated by reference herein. An output screen 2327 is illustrated herein on the upper surface of housing 305 for displaying certain performance or other status information to the user. It is to be specifically noted that the output screen may be of any type, including but not limited to an electrochemical or LCD screen, may be disposable, and may further be provided on any of the embodiments illustrated herein.

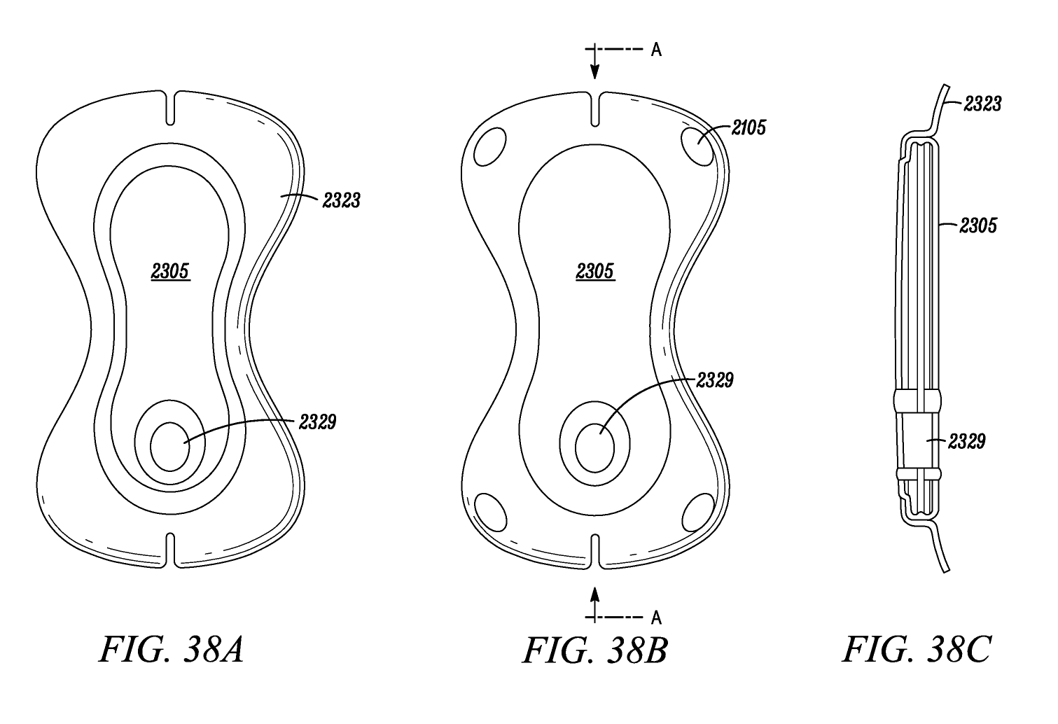

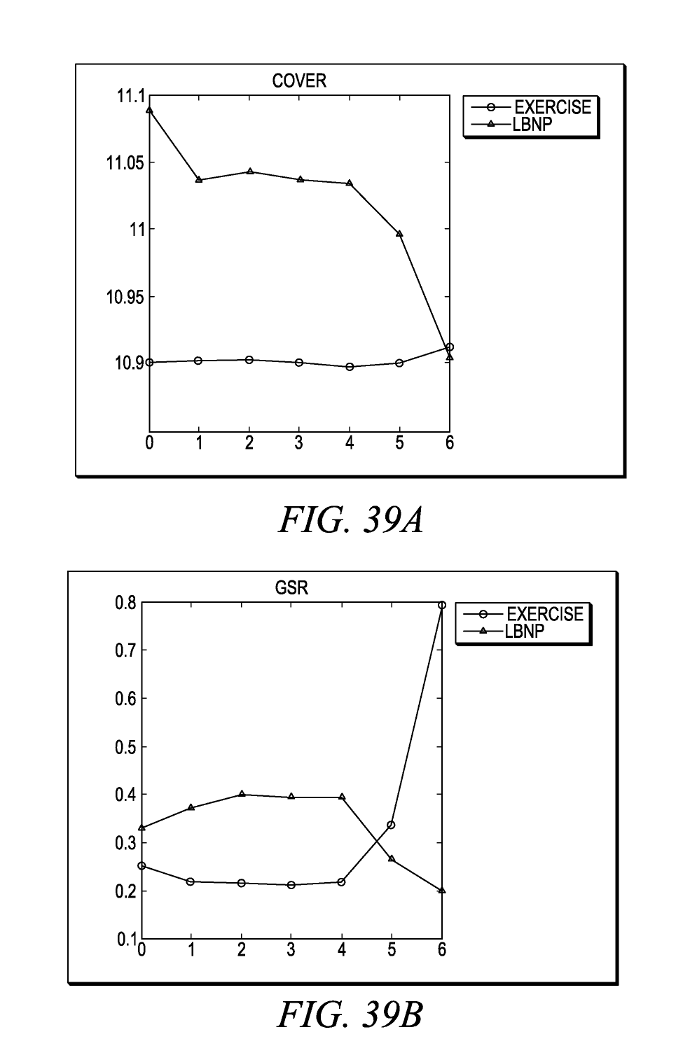

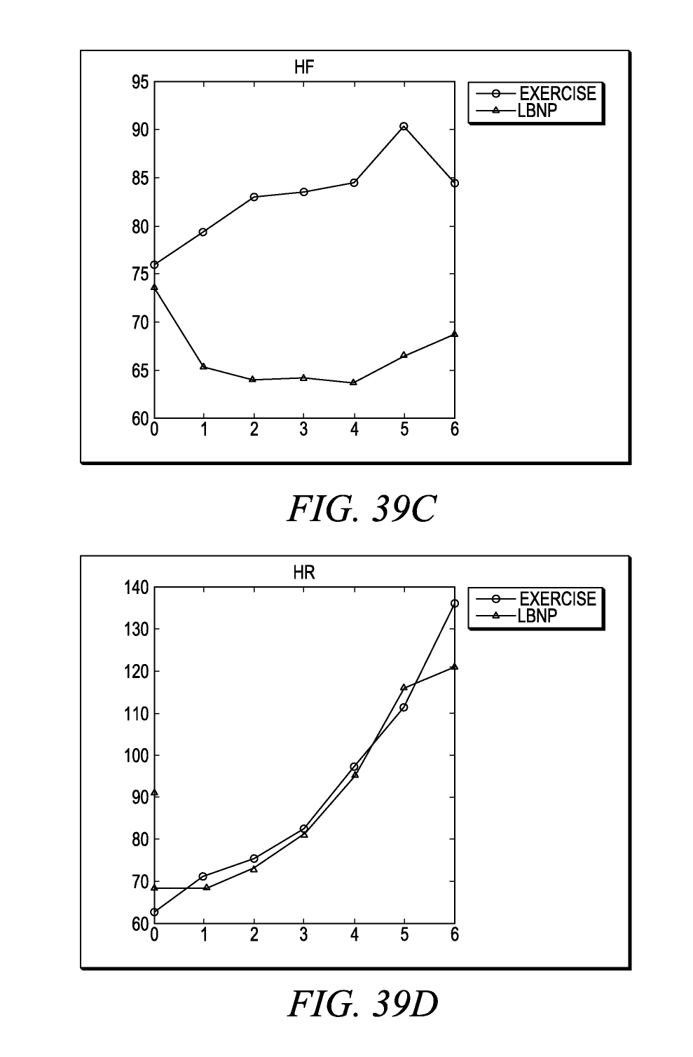

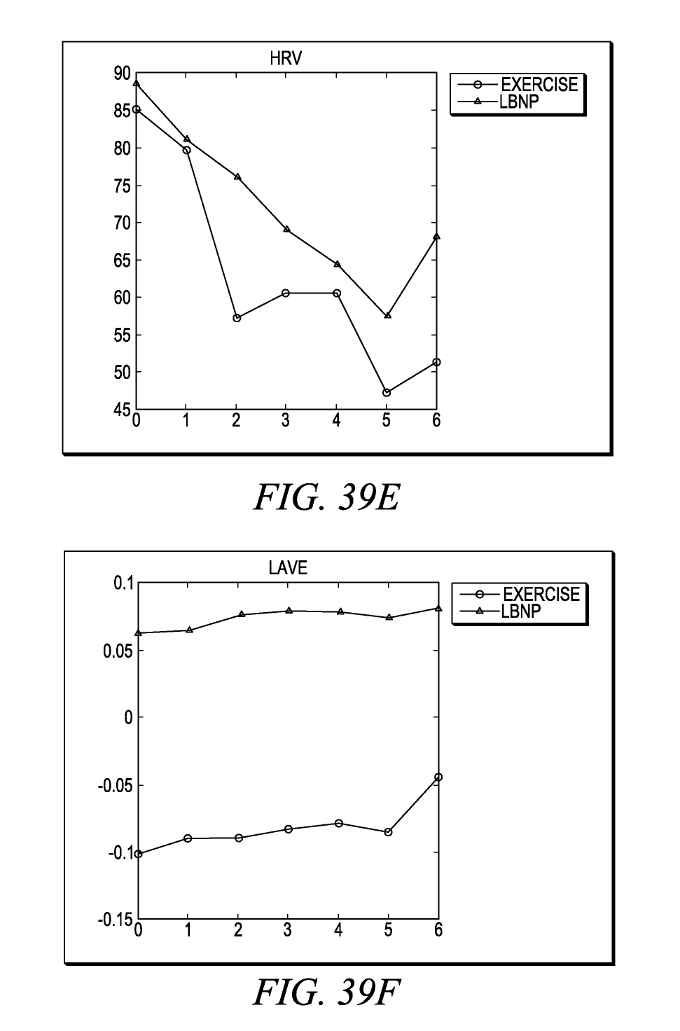

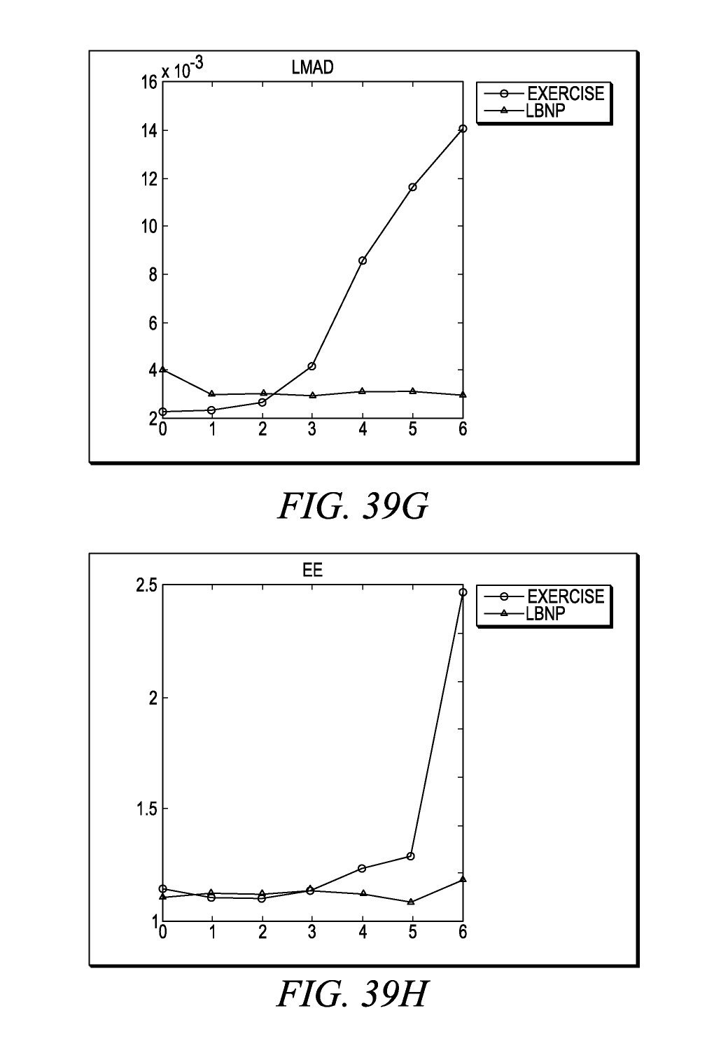

FIGS. 38A-C illustrate yet another embodiment of the device which incorporates a slimmer housing 2305, which is provided with aperture 2329 for functionality which is not relevant hereto. An adhesive support 2323 is mounted semi-equatorially and may contain electrodes 2105, which may also be mounted on the underside of housing 2305. In operation, the housing is affixed to the body through the use of the adhesive provided on adhesive support 2323, which maintains a consistent contact between housing 2305 and/or electrodes 2105 and/or any other relevant sensors contained within housing 2305 and the body. It is to be specifically noted that this adhesive embodiment may be mounted at any point on the human body and is not limited to any particular appendage or location.

An additional aspect of the embodiments illustrated herein is the opportunity to select certain aspects of each device and place the same in disposable segments of the device, as illustrated with particularity in FIG. 37. This may be utilized in conjunction with a permanent, or durable housing 2305 which contains the remaining aspects of the device's functionality. Additionally, the entire device could be rendered in a disposable format, which anticipates a limited continuous wearing time for each system. In this embodiment, as mentioned previously, the entire device might be rendered in a patch-like flexible housing, polymer, film, textile or other support envelope, all of which could be spring-like and which may be mounted anywhere on the body. This includes a textile material which has the electrodes and other electronics interwoven within the material itself, and which exerts sufficient force against the body to maintain appropriate contact for the reception of the signals. Fabrics such as Aracon, a metal clad textile with the strength characteristics of Kevlar, both manufactured by DuPont, are capable of carrying an electrical current or signal therethrough. ElekTex from Eleksen Ltd is a soft textile appropriate for use in clothing or bedding which contains electrodes and/or sensors which can detect movement or pressure. These fabrics could be utilized in combination with the device components in a wearable shirt or other garment which could both sense the appropriate signals as well as provide a network for the interconnection of the various electrical components which could be located at various convenient places within the garment.

The ECG wave form collected from inside any of the equivalence class regions will not necessarily have the shape of a standard ECG wave form. When this is the case, a mapping can be created between a ECG wave form taken within a single equivalence class region and ECG wave forms taken between equivalence class regions. This can be done using the algorithm development process described above, creating a function that warps the within equivalence class region to be clearer when displayed as a standard ECG wave form.

In an additional aspect, the device and method of the present invention utilizes development of mathematic formulas and/or algorithms which correlate the measurement of physiological parameters with oxygen expenditure and oxygen debt. In one embodiment, computational manipulation of these variables equates to a level of OD. This analysis may include a determination of the area under the curve oxygen consumption levels from baseline. The higher the sampling frequency of these parameters, the greater the correlation of the derived measures of OD to traditional measures of OD. The levels of accuracy and precision should enable the measure of OD as determined by this formula to replace traditional measures as determined by such methods of the Bland-Altman analysis.

The technologies allowing for the measurement of certain physiological parameters related to energy expenditure are known in the art. In one embodiment, the measures of traditional oxygen debt correlates can be made amperometrically in a biocompatible matrix in which lactate reacts with specific embedded chemical constituents. This reaction produces an amperometirc response proportional to their concentrations. The biocompatibility of the reaction platform allows for its implant in a variety of biologic environments while maintaining its function. In one aspect, the invention comprises placement of the device directly into tissue and the vasculature. When implanted, the device is inductively powered as described above and data is logged and reported to a remote location. The device may be implanted in tissues for interstitial monitoring, placed within the vasculature (including bone marrow cavity) for real-time systemic blood monitoring and even potentially worn for sampling of subcutaneous fluids. Preexisting algorithms are used to effect diagnosis and subsequent treatments. In one embodiment, the inventive device and method will derive the context of an individual, as explained in detail below. For example, the device will determine that the individual is at rest. A determination that an individual's oxygen consumption or energy expenditure is increasing while the person is inactive or lying down is indicative of the individual entering a critical state.

In an alternative embodiment, the device consists of a wearable device which uses data fusion of various variables as described above in Table 1, including, GSR, heat flux, accelerometer/actigraphy measures, heart rate, skin temperature, skin temperature to ambient temperature differentials, and other measures. Other indicators such as tissue CO₂ levels, tissue hemoglobin oxygen saturation levels, and tissue NADH levels as determined by various methodologies such as optical spectroscopy, and fluorescence, may also be used to determine energy expenditure and then derive oxygen debt, especially when data fusion and computational methods are applied to the data. Some of these technologies could be implantable or wearable in the future.

Since the principles of OD which apply to the body as a whole will also apply to individual organs, it is likely that these methods could be used to predict the outcome of individual organ injury in terms of lifespan or function if oxygen consumption as a function of time could be measured within an organ of interest. This would be especially valuable if the differences between VO₂ of the organ and the systemic circulation could be compared.

These devices such a those made by BodyMedia of Pittsburgh, Pa. use these variables along with techniques of data fusion and algorithms to measure oxygen consumption. However, they have been marketed for physical fitness use and not as a measure of critical body function such as oxygen debt. The novel use of these devices with a new algorithms produce a method to measure oxygen debt in real time by subtracting current oxygen consumption from basal levels and cumulating these results.

The present invention allows for the measurement of the described physiological factors in real time and transmitted to the user or to remote sites for monitoring and decision making, as described previously. The foregoing device and method would be particularly used in mass casualty situations in both the civilian and combat settings. When coupled with other indicators such as heart rate variability, blood pressure, respiratory rate and other noninvasive measures, a powerful predictive indicator of outcome and a guide to treatment can be envisioned.

Both animal and clinical data support the findings that first, late outcome is strongly related to both the severity and duration of shock, and second, oxygen debt and its metabolic surrogates are the best predictors of outcome. To understand the concept of oxygen debt, it is useful to describe the relationship between oxygen delivery and oxygen consumption during normal perfusion and in shock. In the normal healthy subject, whole body oxygen consumption is independent of cardiac output, and hence DO₂, because of the ability of the tissues to modulate oxygen extraction from the blood at the level of the microcirculation. However, if DO₂ is decreased below a certain threshold, critical oxygen delivery DO_{2crit}, extraction is no longer adequate and VO₂ declines in proportion to the reduction in DO₂; ischemic metabolic insufficiency then follows. A marker of this insufficiency is the increase in the concentration of metabolites, such as lactate, in the peripheral blood.

When DO₂ is reduced below DO_{2crit}, an oxygen deficit is incurred because the amount of oxygen demanded by the tissues is inadequately matched by supply; this is the standard definition of shock. Therefore oxygen deficit can be calculated as the difference between baseline “normal” oxygen consumption VO₂, and the VO₂ measured at a given time during the shock period. However, because there is a significant associated time dimension, shock cannot be evaluated merely by the oxygen deficit “snapshot” of perfusion status at any one time; the shock state must account for the amount of deficit accumulated over time from the point of injury. Deficit accumulated over time is debt. In other words, oxygen debt is the accumulation of multiple oxygen deficits over time and thus represents the sum of all deficits incurred. As an example, suppose that baseline VO₂, an estimate of tissue oxygen demand, is 200 mL/min, and is followed by a reduction in VO₂ by slightly more than one-third to 134 mL/min. Because oxygen deficit is the change in VO₂ from baseline, oxygen deficit is therefore equal to the difference between baseline VO₂ (VO_{2,0}) and the VO₂ at this new time point t, or

Oxygen deficit=VO_{2,0}−VO_{2,t}

In this example, the reduction in VO₂ results in an oxygen deficit of (200−134)=66 mL/min. If this deficit is sustained for a period of one hour, the resulting oxygen debt will be equal to the product of the oxygen deficit integrated over time (66 mL/min×60 min), or 3.96 L.

When data is retrieved from the apparatus, the system may provide a semi-automated interface. The system is provided with the capability to communicate with the apparatus both wirelessly and with a wired USB connection. The system prompts the user to select the mode of communication before the retrieval of data. It is contemplated that the most common usage model may be wireless retrieval. If wireless retrieval is used, a wired connection could be used primarily for field upgrades of the firmware in the device. Each apparatus is associated with a particular user and the apparatus is personalized so that it cannot be interchanged between different users.

The system will use the data collected by the device for calculating the total OD. This value is calculated using an algorithm contained within the software. The database stores the minute-by-minute estimates of OD values, the number of steps, the amount of time the device was functioning, oxygen consumption and blood glucose and/or lactate levels values.

The feedback provided by the device allowing for the continuous measurement of certain physiological parameter levels is helpful in diagnosis and guiding treatment to prolong survival. For example, tight regulation of systemic glucose levels have been demonstrated to be a factor in improving outcomes from a variety of critical illness an injuries. The ability to monitor these levels allows for continuous adjustment of caloric intake and insulin or other hormone administration to prevent wide swings in systemic glucose levels. These values in turn provide the health care provider with information that can be continuously used to evaluate the severity of injury or illness, the effects of treatment, and finally to predict outcome.

It will be clear to one skilled in the art that the description that the above-described method and device, while described for the specific determination of oxygen debt as a result of shock, need not be limited to that particular event. The process could also be adapted and applied without limitation to other disease states including but not limited to:

### 1) Trauma

### 2) Congestive Heart Failure

### 3) Sepsis

4) Organ transplant
5) Cardiopulmonary bypass surgery

### 6) Diabetes

7) Individuals at risk for critical illness and injury
8) Combat setting
9) Mass casualties

### 10) Nursing Home Patients

The system will use the data collected by the armband for estimating the total energy expenditure. This value is calculated using an algorithm contained within the software. There are several calculations that may be used to convert oxygen consumption to energy expenditure or calories burned. The most widely used methods is based on the “Lusk equation”. This equation used VO₂ and VCO₂, expended carbon dioxide. First a term called RQ or Respiratory Quotient, also sometime called RER, Respiratory Exchange Ratio, is calculated using the following equation:

RQ=VCO₂/VO₂

If RQ is less than 0.707, RQ is set to 0.707, and if RQ is greater than 1, RQ is set to 1. Thus, the RQ may be in the range between 0.707 and 1. A table called the “Lusk Table” is used then to convert RQ values to Kcal values. Below is one illustration of the Lusk Table:

TABLE 5

| RQ | Kcal |
|---|---|
| 0.707 | 4.6862 |
| 0.75 | 4.7387 |
| 0.8 | 4.8008 |
| 0.85 | 4.8605 |
| 0.9 | 4.9226 |
| 0.95 | 4.9847 |
| 1 | 5.0468 |

A linear interpolation is used to estimate a corresponding Kcal value for an interim value of RQ.

It is not possible to calculate the RQ term if the Value of VCO₂ is not available. In this case, the following equation is used to estimate the KCals using the VO2 measurement (ACSM 6^{th} edition p 300).

VO₂(in L/min)*5=Kcal/min

The database stores the minute-by-minute estimates of the energy expenditure values, the number of steps, the amount of time the apparatus was worn, the active energy expenditure values, the user's habits, which, in the preferred embodiment are stored as typical hourly non-physically active energy expenditure, their reported exercise while not wearing the apparatus, and the time spent actively.

In addition to monitoring of physiological and contextual parameters, environmental parameters may also be monitored to determine the effect on the user. These parameters may include ozone, pollen count, and humidity.

The system may also include a reporting feature to provide a summary of the VO₂ and OD levels or oxygen debt for a period of time. The user may be provided with an interface to visualize graphically and analyze these numbers. The input values for the oxygen debt calculation are the lactate levels based on the data collected by the device. The user may be provided with this information both in an equation form and visually. Shortcuts are provided for commonly used summary time periods, such as daily, yesterday, last 7 days, last 30 days and since beginning, etc. The information my be provided to the user in a continuous or intermittent form.

The report can also be customized in various ways including what the user has asked to see in the past or what the user actually has done. The reports may be customized by third party specifications or by user selection. The user may ask to see a diary of past feedback to see the type of feedback previously received. One skilled in the art will recognize that the reports can be enhanced in all the ways that the feedback engine can be enhanced and can be viewed as an extension of the feedback engine.

With respect to the calculation of OD, the armband sensor device continuously measures a person's energy expenditure. During the day the human body is continuously burning calories. The minimal rate that a human body expends energy is called resting metabolic rate, or RMR. For an average person, the daily RMR is about 1500 calories. It is more for larger people.

Energy expenditure is different than RMR because a person knows throughout the day how many calories have been burned so far, both at rest and when active. At the time when the user views energy expenditure information, two things are known. First, the caloric burn of that individual from midnight until that time of day, as recorded by armband sensor device. Second, that user's RMR from the current time until the end of the day. The sum of these numbers is a prediction of the minimum amount of calories that the user expends during the day.

This estimate may be improved by applying a multiplicative factor to RMR. A person's lifestyle contributes greatly to the amount of energy they expend. A sedentary person who does not exercise burns calories only slightly more than those consumed by their RMR. An athlete who is constantly active burns significantly more calories than RMR. These lifestyle effects on RMR may be estimated as multiplicative factors to RMR ranging from 1.1 for a sedentary person to 1.7 for an athlete. This multiplicative factor may also calculated from an average measurement of the person's wear time based on the time of day or the time of year, or it may be determined from information a user has entered in date or time management program, as described above. Using such a factor greatly improves the predictive nature of the estimated daily expenditure for an individual.

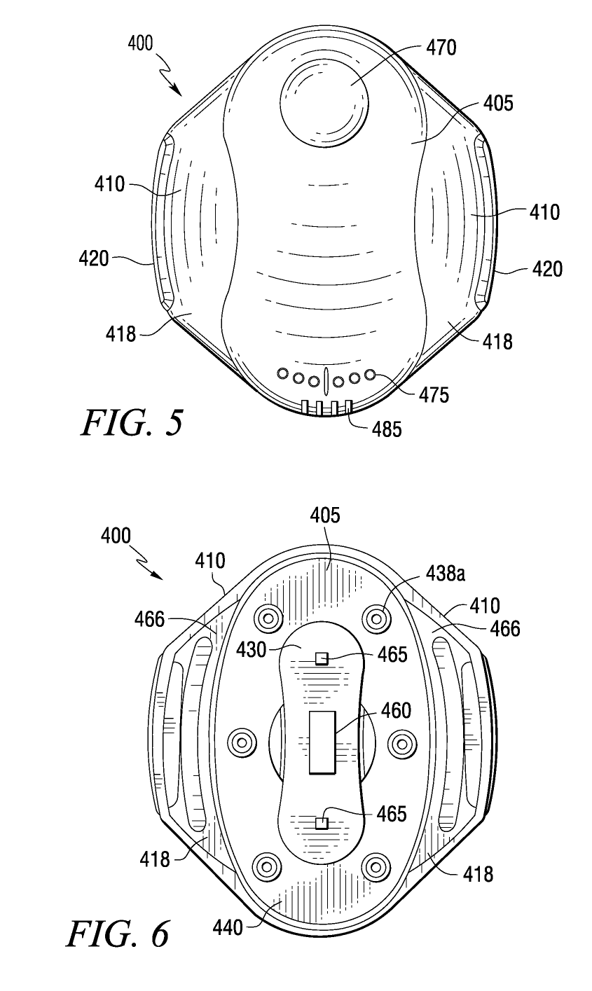

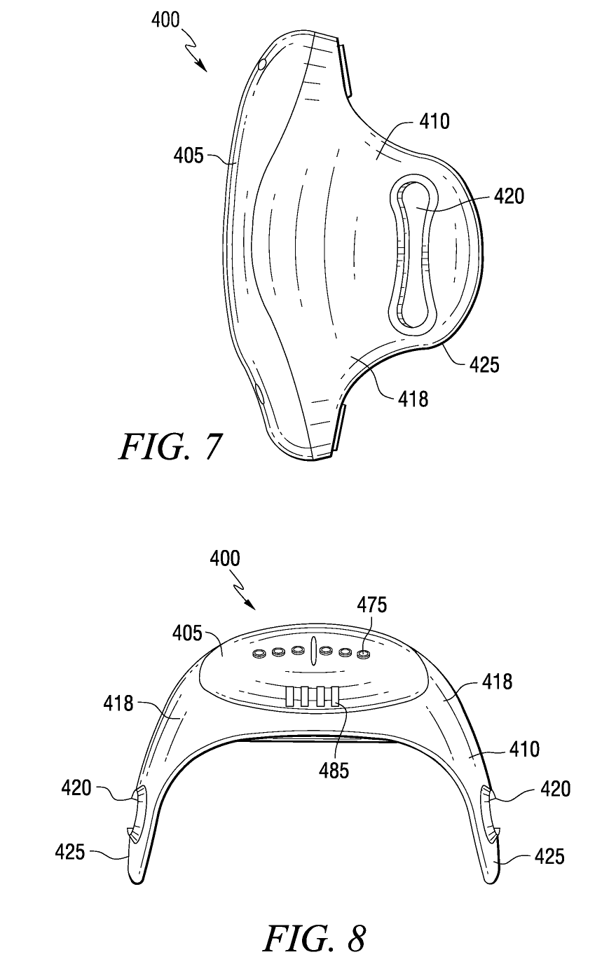

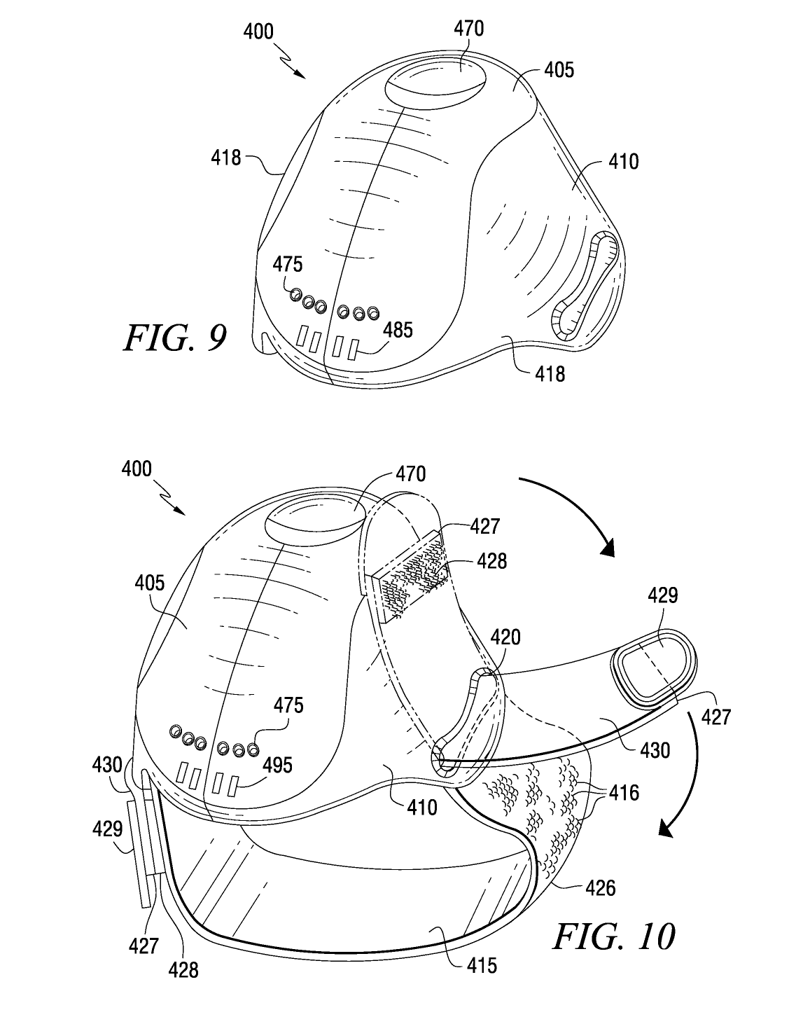

A specific embodiment of sensor device 10 is shown which is in the form of an armband adapted to be worn by an individual on his or her upper arm, between the shoulder and the elbow, as illustrated in FIGS. 5-11. Although a similar sensor device may be worn on other parts of the individual's body, these locations have the same function for single or multi-sensor measurements and for the automatic detection and/or identification of the user's activities or state. For the purpose of this disclosure, the specific embodiment of sensor device 10 shown in FIGS. 5-10 will, for convenience, be referred to as armband sensor device 400. Armband sensor device 400 includes computer housing 405, flexible wing body 410, and, as shown in FIG. 10, elastic strap 415. Computer housing 405 and flexible wing body 410 are preferably made of a flexible urethane material or an elastomeric material such as rubber or a rubber-silicone blend by a molding process. Flexible wing body 410 includes first and second wings 418 each having a thru-hole 420 located near the ends 425 thereof. First and second wings 418 are adapted to wrap around a portion of the wearer's upper arm.

Elastic strap 415 is used to removably affix armband sensor device 400 to the individual's upper arm. As seen in FIG. 10, bottom surface 426 of elastic strap 415 is provided with velcro loops 416 along a portion thereof. Each end 427 of elastic strap 415 is provided with velcro hook patch 428 on bottom surface 426 and pull tab 429 on top surface 430. A portion of each pull tab 429 extends beyond the edge of each end 427.

In order to wear armband sensor device 400, a user inserts each end 427 of elastic strap 415 into a respective thru-hole 420 of flexible wing body 410. The user then places his arm through the loop created by elastic strap 415, flexible wing body 410 and computer housing 405. By pulling each pull tab 429 and engaging velcro hook patches 428 with velcro loops 416 at a desired position along bottom surface 426 of elastic strap 415, the user can adjust elastic strap 415 to fit comfortably. Since velcro hook patches 428 can be engaged with velcro loops 416 at almost any position along bottom surface 426, armband sensor device 400 can be adjusted to fit arms of various sizes. Also, elastic strap 415 may be provided in various lengths to accommodate a wider range of arm sizes. As will be apparent to one of skill in the art, other means of fastening and adjusting the size of elastic strap may be used, including, but not limited to, snaps, buttons, or buckles. It is also possible to use two elastic straps that fasten by one of several conventional means including velcro, snaps, buttons, buckles or the like, or merely a single elastic strap affixed to wings 418.

Alternatively, instead of providing thru-holes 420 in wings 418, loops having the shape of the letter D, not shown, may be attached to ends 425 of wings 418 by one of several conventional means. For example, a pin, not shown, may be inserted through ends 425, wherein the pin engages each end of each loop. In this configuration, the D-shaped loops would serve as connecting points for elastic strap 415, effectively creating a thru-hole between each end 425 of each wing 418 and each loop.

As shown in FIG. 11, which is an exploded view of armband sensor device 400, computer housing 405 includes a top portion 435 and a bottom portion 440. Contained within computer housing 405 are printed circuit board or PCB 445, rechargeable battery 450, preferably a lithium ion battery, and vibrating motor 455 for providing tactile feedback to the wearer, such as those used in pagers, suitable examples of which are the Model 12342 and 12343 motors sold by MG Motors Ltd. of the United Kingdom.

Top portion 435 and bottom portion 440 of computer housing 405 sealingly mate along groove 436 into which O-ring 437 is fit, and may be affixed to one another by screws, not shown, which pass through screw holes 438a and stiffeners 438b of bottom portion 440 and apertures 439 in PCB 445 and into threaded receiving stiffeners 451 of top portion 435. Alternately, top portion 435 and bottom portion 440 may be snap fit together or affixed to one another with an adhesive. Preferably, the assembled computer housing 405 is sufficiently water resistant to permit armband sensor device 400 to be worn while swimming without adversely affecting the performance thereof.

As can be seen in FIG. 6, bottom portion 440 includes, on a bottom side thereof, a raised platform 430. Affixed to raised platform 430 is heat flow or flux sensor 460, a suitable example of which is the micro-foil heat flux sensor sold by RdF Corporation of Hudson, N.H. Heat flux sensor 460 functions as a self-generating thermopile transducer, and preferably includes a carrier made of a polyamide film. Bottom portion 440 may include on a top side thereof, that is on a side opposite the side to which heat flux sensor 460 is affixed, a heat sink, not shown, made of a suitable metallic material such as aluminum. Also affixed to raised platform 430 are GSR sensors 465, preferably comprising electrodes formed of a material such as conductive carbonized rubber, gold or stainless steel. Although two GSR sensors 465 are shown in FIG. 6, it will be appreciated by one of skill in the art that the number of GSR sensors 465 and the placement thereof on raised platform 430 can vary as long as the individual GSR sensors 465, i.e., the electrodes, are electrically isolated from one another. By being affixed to raised platform 430, heat flux sensor 460 and GSR sensors 465 are adapted to be in contact with the wearer's skin when armband sensor device 400 is worn. Bottom portion 440 of computer housing 405 may also be provided with a removable and replaceable soft foam fabric pad, not shown, on a portion of the surface thereof that does not include raised platform 430 and screw holes 438a. The soft foam fabric is intended to contact the wearer's skin and make armband sensor device 400 more comfortable to wear.

Electrical coupling between heat flux sensor 460, GSR sensors 465, and PCB 445 may be accomplished in one of various known methods. For example, suitable wiring, not shown, may be molded into bottom portion 440 of computer housing 405 and then electrically connected, such as by soldering, to appropriate input locations on PCB 445 and to heat flux sensor 460 and GSR sensors 465. Alternatively, rather than molding wiring into bottom portion 440, thru-holes may be provided in bottom portion 440 through which appropriate wiring may pass. The thru-holes would preferably be provided with a water tight seal to maintain the integrity of computer housing 405.

Rather than being affixed to raised platform 430 as shown in FIG. 6, one or both of heat flux sensor 460 and GSR sensors 465 may be affixed to the inner portion 466 of flexible wing body 410 on either or both of wings 418 so as to be in contact with the wearer's skin when armband sensor device 400 is worn. In such a configuration, electrical coupling between heat flux sensor 460 and GSR sensors 465, whichever the case may be, and the PCB 445 may be accomplished through suitable wiring, not shown, molded into flexible wing body 410 that passes through one or more thru-holes in computer housing 405 and that is electrically connected, such as by soldering, to appropriate input locations on PCB 445. Again, the thru-holes would preferably be provided with a water tight seal to maintain the integrity of computer housing 405. Alternatively, rather than providing thru-holes in computer housing 405 through which the wiring passes, the wiring may be captured in computer housing 405 during an overmolding process, described below, and ultimately soldered to appropriate input locations on PCB 445.

As shown in FIGS. 5, 9, 10 and 11, computer housing 405 includes a button 470 that is coupled to and adapted to activate a momentary switch 585 on PCB 445. Button 470 may be used to activate armband sensor device 400 for use, to mark the time an event occurred or to request system status information such as battery level and memory capacity. When button 470 is depressed, momentary switch 585 closes a circuit and a signal is sent to processing unit 490 on PCB 445. Depending on the time interval for which button 470 is depressed, the generated signal triggers one of the events just described. Computer housing 405 also includes LEDs 475, which may be used to indicate battery level or memory capacity or to provide visual feedback to the wearer. Rather than LEDs 475, computer housing 405 may also include a liquid crystal display or LCD to provide battery level, memory capacity or visual feedback information to the wearer. Battery level, memory capacity or feedback information may also be given to the user tactily or audibly. The circuit is place inside housing 405 of armband body monitoring device 400, and the various electrodes and sensors identified herein are electrically connected thereto, as will be apparent to one skilled in the art. CPU 165 of the circuit would, in this embodiment, preferably be the processing unit forming part of the armband body monitoring device circuitry described in U.S. Pat. No. 6,605,038 and U.S. application Ser. No. 10/682,293, the specifications of both which are hereby incorporated by reference.

Armband sensor device 400 may be adapted to be activated for use, that is collecting data, when either of GSR sensors 465 or heat flux sensor 460 senses a particular condition that indicates that armband sensor device 400 has been placed in contact with the user's skin. Also, armband sensor device 400 may be adapted to be activated for use when one or more of heat flux sensor 460, GSR sensors 465, accelerometer 495 or 550, or any other device in communication with armband sensor device 400, alone or in combination, sense a particular condition or conditions that indicate that the armband sensor device 400 has been placed in contact with the user's skin for use. At other times, armband sensor device 400 would be deactivated, thus preserving battery power.

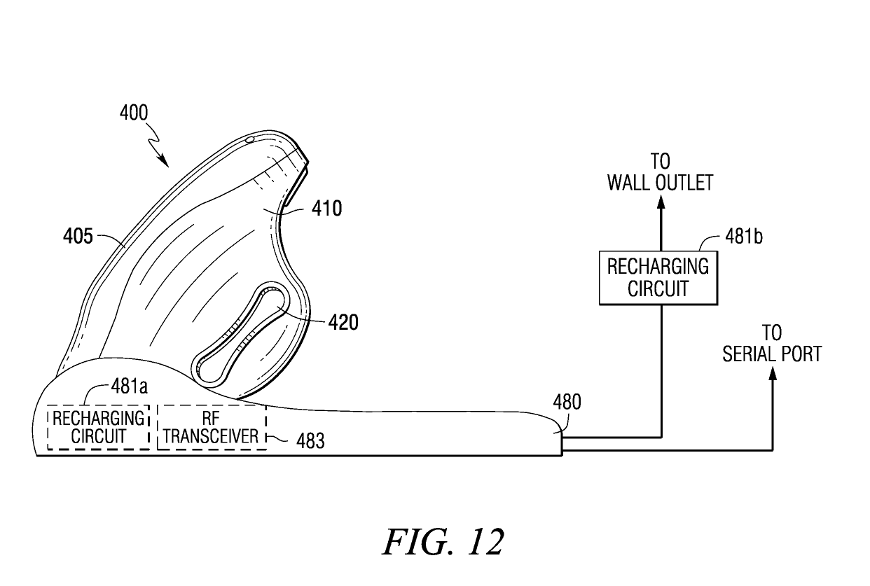

Computer housing 405 is adapted to be coupled to a battery recharger unit 480 shown in FIG. 12 for the purpose of recharging rechargeable battery 450. Computer housing 405 includes recharger contacts 485, shown in FIGS. 5, 9, 10 and 11, that are coupled to rechargeable battery 450. Recharger contracts 485 may be made of a material such as brass, gold or stainless steel, and are adapted to mate with and be electrically coupled to electrical contacts, not shown, provided in battery recharger unit 480 when armband sensor device 400 is placed therein. The electrical contacts provided in battery recharger unit 480 may be coupled to recharging circuit 481a provided inside battery recharger unit 480. In this configuration, recharging circuit 481 would be coupled to a wall outlet, such as by way of wiring including a suitable plug that is attached or is attachable to battery recharger unit 480. Alternatively, electrical contacts 480 may be coupled to wiring that is attached to or is attachable to battery recharger unit 480 that in turn is coupled to recharging circuit 481b external to battery recharger unit 480. The wiring in this configuration would also include a plug, not shown, adapted to be plugged into a conventional wall outlet.

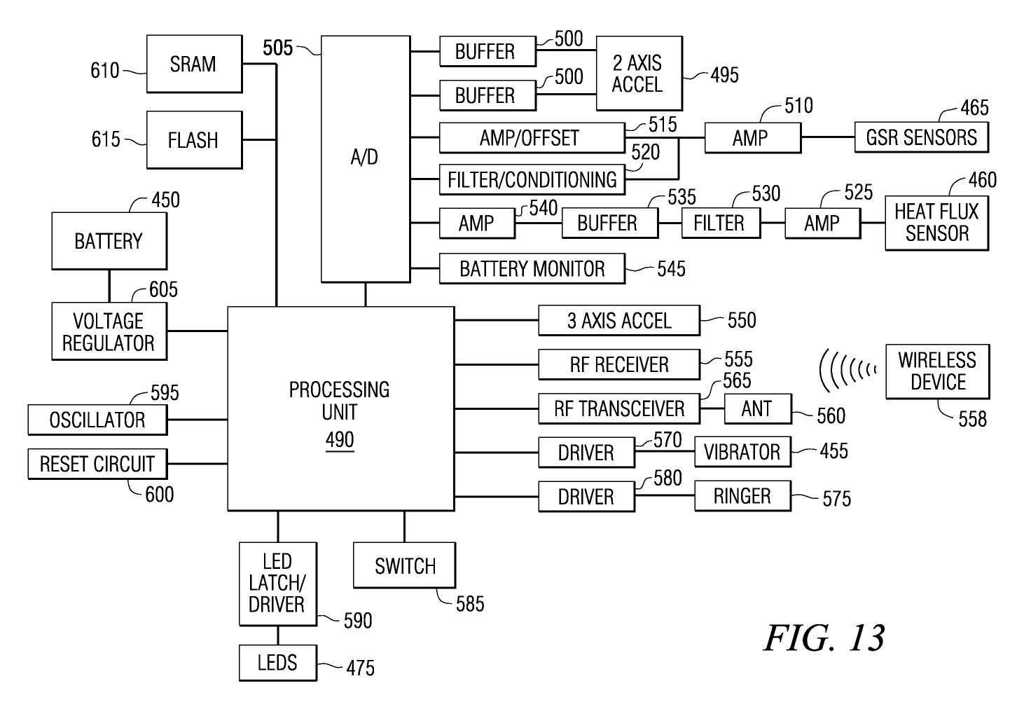

Also provided inside battery recharger unit 480 is RF transceiver 483 adapted to receive signals from and transmit signals to RF transceiver 565 provided in computer housing 405 and shown in FIG. 12. RF transceiver 483 is adapted to be coupled, for example by a suitable cable, to a serial port, such as an RS 232 port or a USB port, of a device such as personal computer 35 shown in FIG. 1. Thus, data may be uploaded from and downloaded to armband sensor device 400 using RF transceiver 483 and RF transceiver 565. It will be appreciated that although RF transceivers 483 and 565 are shown in FIGS. 12 and 13, other forms of wireless transceivers may be used, such as infrared transceivers. Alternatively, computer housing 405 may be provided with additional electrical contacts, not shown, that would be adapted to mate with and be electrically coupled to additional electrical contacts, not shown, provided in battery recharger unit 480 when armband sensor device 400 is placed therein. The additional electrical contacts in the computer housing 405 would be coupled to the processing unit 490 and the additional electrical contacts provided in battery recharger unit 480 would be coupled to a suitable cable that in turn would be coupled to a serial port, such as an RS R32 port or a USB port, of a device such as personal computer 35. This configuration thus provides an alternate method for uploading of data from and downloading of data to armband sensor device 400 using a physical connection. In one non-limiting example, the connection may be through a USB connector, the GSR or ECG electrodes, wireless data or wireless power.

FIG. 13 is a schematic diagram that shows the system architecture of armband sensor device 400, and in particular each of the components that is either on or coupled to PCB 445.

As shown in FIGS. 10, 11 and 13, PCB 445 includes processing unit 490, which may be a microprocessor, a microcontroller, or any other processing device that can be adapted to perform the functionality described herein. Processing unit 490 is adapted to provide all of the functionality described in connection with microprocessor 20 shown in FIG. 2. PCB 445 also has thereon a two-axis accelerometer 495, a suitable example of which is the Model ADXL210 accelerometer sold by Analog Devices, Inc. of Norwood, Mass. Two-axis accelerometer 495 is preferably mounted on PCB 445 at an angle such that its sensing axes are offset at an angle substantially equal to 45 degrees from the longitudinal axis of PCB 445 and thus the longitudinal axis of the wearer's arm when armband sensor device 400 is worn. The longitudinal axis of the wearer's arm refers to the axis defined by a straight line drawn from the wearer's shoulder to the wearer's elbow. The output signals of two-axis accelerometer 495 are passed through buffers 500 and input into analog to digital converter 505 that in turn is coupled to processing unit 490. GSR sensors 465 are coupled to amplifier 510 on PCB 445. Amplifier 510 provides amplification and low pass filtering functionality, a suitable example of which is the Model AD8544 amplifier sold by Analog Devices, Inc. of Norwood, Mass. The amplified and filtered signal output by amplifier 510 is input into amp/offset 515 to provide further gain and to remove any bias voltage and into filter/conditioning circuit 520, which in turn are each coupled to analog to digital converter 505. Heat flux sensor 460 is coupled to differential input amplifier 525, such as the Model INA amplifier sold by Burr-Brown Corporation of Tucson, Ariz., and the resulting amplified signal is passed through filter circuit 530, buffer 535 and amplifier 540 before being input to analog to digital converter 505. Amplifier 540 is configured to provide further gain and low pass filtering, a suitable example of which is the Model AD8544 amplifier sold by Analog Devices, Inc. of Norwood, Mass. PCB 445 also includes thereon a battery monitor 545 that monitors the remaining power level of rechargeable battery 450. Battery monitor 545 preferably comprises a voltage divider with a low pass filter to provide average battery voltage. When a user depresses button 470 in the manner adapted for requesting battery level, processing unit 490 checks the output of battery monitor 545 and provides an indication thereof to the user, preferably through LEDs 475, but also possibly through vibrating motor 455 or ringer 575. An LCD may also be used.

PCB 445 may include three-axis accelerometer 550 instead of or in addition to two-axis accelerometer 495. The three-axis accelerometer outputs a signal to processing unit 490. A suitable example of three-axis accelerometer is the μPAM product sold by I.M. Systems, Inc. of Scottsdale, Ariz. Three-axis accelerometer 550 is preferably tilted in the manner described with respect to two-axis accelerometer 495.

PCB 445 also includes RF receiver 555 that is coupled to processing unit 490. RF receiver 555 may be used to receive signals that are output by another device capable of wireless transmission, shown in FIG. 13 as wireless device 558, worn by or located near the individual wearing armband sensor device 400. Located near as used herein means within the transmission range of wireless device 558. For example, wireless device 558 may be a chest mounted heart rate monitor such as the Tempo product sold by Polar Electro of Oulu, Finland. Using such a heart rate monitor, data indicative of the wearer's heart rate can be collected by armband sensor device 400. Antenna 560 and RF transceiver 565 are coupled to processing unit 490 and are provided for purposes of uploading data to central monitoring unit 30 and receiving data downloaded from central monitoring unit 30. RF transceiver 565 and RF receiver 555 may, for example, employ Bluetooth technology as the wireless transmission protocol. Also, other forms of wireless transmission may be used, such as infrared transmission.

Vibrating motor 455 is coupled to processing unit 490 through vibrator driver 570 and provides tactile feedback to the wearer. Similarly, ringer 575, a suitable example of which is the Model SMT916A ringer sold by Projects Unlimited, Inc. of Dayton, Ohio, is coupled to processing unit 490 through ringer driver 580, a suitable example of which is the Model MMBTA14 CTI darlington transistor driver sold by Motorola, Inc. of Schaumburg, Ill., and provides audible feedback to the wearer. Feedback may include, for example, celebratory, cautionary and other threshold or event driven messages, such as when a wearer reaches a level of calories burned during a workout.

Also provided on PCB 445 and coupled to processing unit 490 is momentary switch 585. Momentary switch 585 is also coupled to button 470 for activating momentary switch 585. LEDs 475, used to provide various types of feedback information to the wearer, are coupled to processing unit 490 through LED latch/driver 590.

Oscillator 595 is provided on PCB 445 and supplies the system clock to processing unit 490. Reset circuit 600, accessible and triggerable through a pin-hole in the side of computer housing 405, is coupled to processing unit 490 and enables processing unit 490 to be reset to a standard initial setting.

Rechargeable battery 450, which is the main power source for the armband sensor device 400, is coupled to processing unit 490 through voltage regulator 605. Finally, memory functionality is provided for armband sensor device 400 by SRAM 610, which stores data relating to the wearer of armband sensor device 400, and flash memory 615, which stores program and configuration data, provided on PCB 445. SRAM 610 and flash memory 615 are coupled to processing unit 490 and each preferably have at least 512K of memory.

In manufacturing and assembling armband sensor device 400, top portion 435 of computer housing 405 is preferably formed first, such as by a conventional molding process, and flexible wing body 410 is then overmolded on top of top portion 435. That is, top portion 435 is placed into an appropriately shaped mold, i.e., one that, when top portion 435 is placed therein, has a remaining cavity shaped according to the desired shape of flexible wing body 410, and flexible wing body 410 is molded on top of top portion 435. As a result, flexible wing body 410 and top portion 435 will merge or bond together, forming a single unit. Alternatively, top portion 435 of computer housing 405 and flexible wing body 410 may be formed together, such as by molding in a single mold, to form a single unit. The single unit however formed may then be turned over such that the underside of top portion 435 is facing upwards, and the contents of computer housing 405 can be placed into top portion 435, and top portion 435 and bottom portion 440 can be affixed to one another. As still another alternative, flexible wing body 410 may be separately formed, such as by a conventional molding process, and computer housing 405, and in particular top portion 435 of computer housing 405, may be affixed to flexible wing body 410 by one of several known methods, such as by an adhesive, by snap-fitting, or by screwing the two pieces together. Then, the remainder of computer housing 405 would be assembled as described above. It will be appreciated that rather than assembling the remainder of computer housing 405 after top portion 435 has been affixed to flexible wing body 410, the computer housing 405 could be assembled first and then affixed to flexible wing body 410.

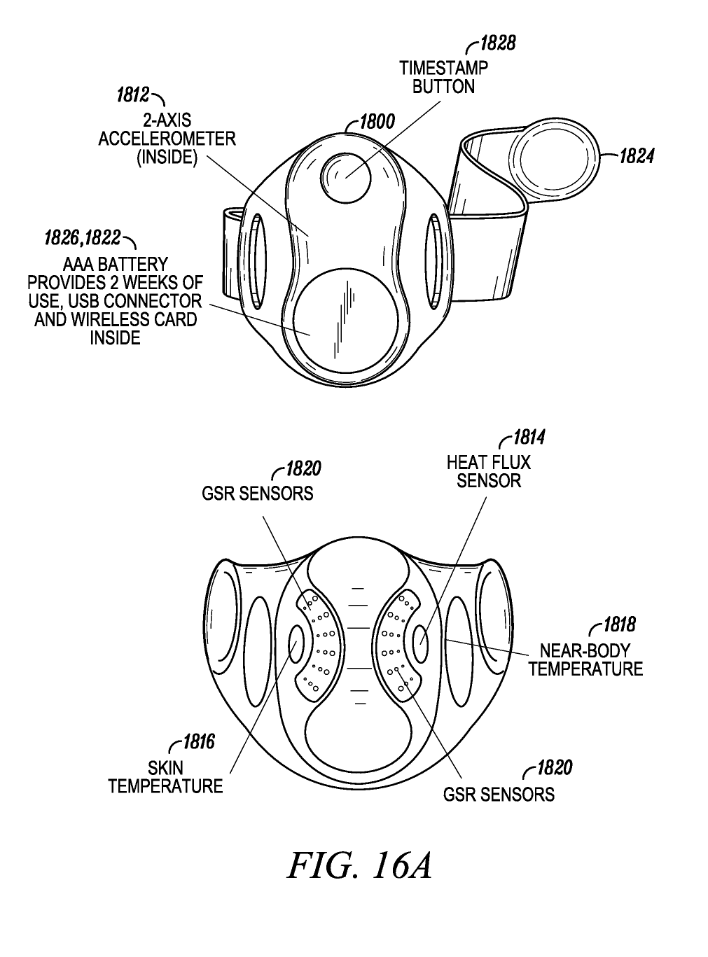

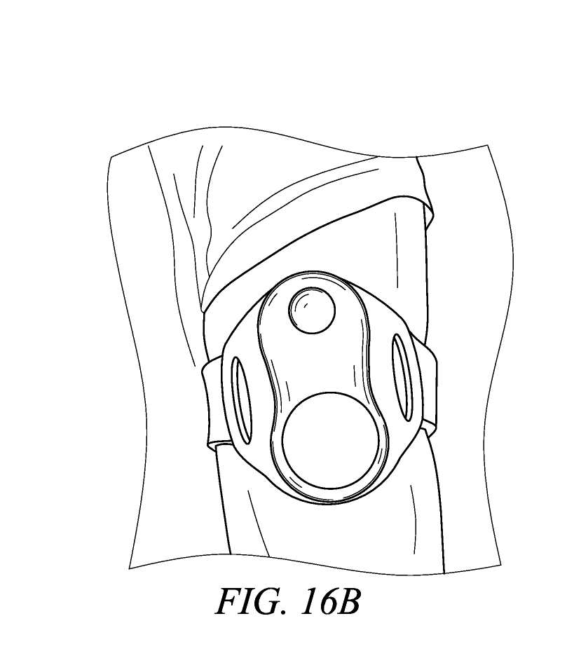

An alternative embodiment of the device of the invention will now be described. Discussed below is the BodyMedia SenseWear®PRO3 Armband. The device, shown in FIGS. 16A and 16B, is worn on the upper arm. The band uses five sensors: a two-axis accelerometer tracks the movement of the upper arm and body and provides information about body position. A heat-flux sensor 1814 measures the amount of heat being dissipated by the body by measuring the heat loss along a thermally conductive path between the skin and a vent on the side of the armband Skin temperature 1816 and near-armband temperature 1818 are also measured by sensitive thermistors.

Armband 1824 also measures galvanic skin response or GSR 1820 which varies due to sweating and emotional stimuli. Armband 1824 also contains a transceiver radio or a type commonly known to those skilled in the art and USB port 1822, allowing wireless transmission and communication as well as wired downloading of data. The armband contains a button 1829 to be used to time stamp events, as described previously. Each sensor is sampled 32 times per second, and data is tracked over a period of time (typically a minute but this can be adjusted through software). Currently, 41 different features of this multi-dimensional raw data stream are gathered as separate channels. For example, the variance of the heat flux is a channel, as is the average of the heat flux values. Some channels are fairly standard features, e.g. standard deviation, and others are complex proprietary algorithms. Then typically, these summary features for each epoch are stored and the raw data discarded to save memory.

The system collects physiological data on a continuous basis from the person wearing the sensor system. Data obtained is conditioned, analyzed, and stored within the device and can later be transferred electronically by direct or wireless connection to a computer, where it is analyzed and interpreted by a comprehensive suite of algorithms to reveal key physiological measures of interest such as energy expenditure or oxygen consumption, sleep, stress, or physical activity. FIG. 16B illustrates the armband as worn on the arm of a subject.

The sensor device 400 includes a 2.4 GHz wireless technology that allows the armband to communicate securely and wirelessly with other devices including computing devices display devices such as watches and kiosks, and other medical devices such as blood glucose meters, weight scales, blood pressure cuffs, and pulse oximetry meters. These devices are enabled with a transceiver, allowing them to communicate with the armband and the measurements are stored in the armband along with the data it records itself. All of the recorded data can then be transmitted to a PC via a wireless communicator that connects to USB port on the PC. Alternatively, the data can be uploaded to a web-server via a wireless gateway which contains either a standard or cellular modem, depending on the application.

This same algorithm development process as described above was used to develop the algorithms disclosed above for detecting heart beats, for determining heart rate, and for estimating heart rate in the presence of noise, described previously. It will be clear to one skilled in the art that this same process could be utilized to both incorporate other sensors to improve the measurement of heart related parameters or to incorporate heart related parameters into the measurement of other physiological parameters such as energy expenditure.

### EXAMPLES

### Example 1

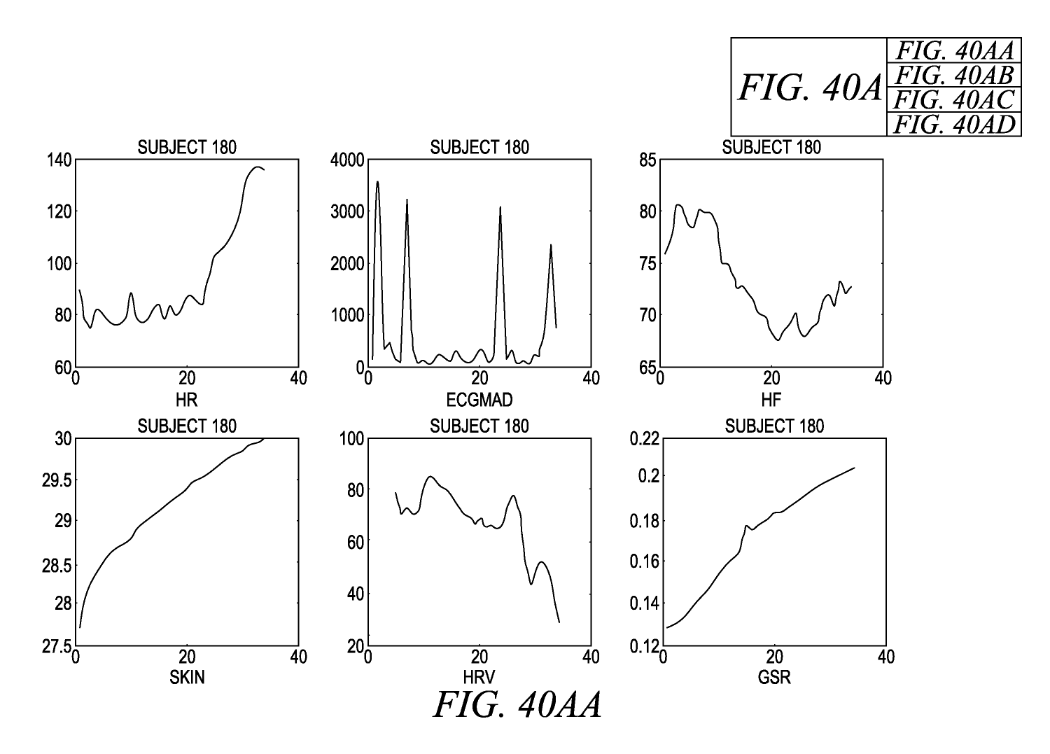

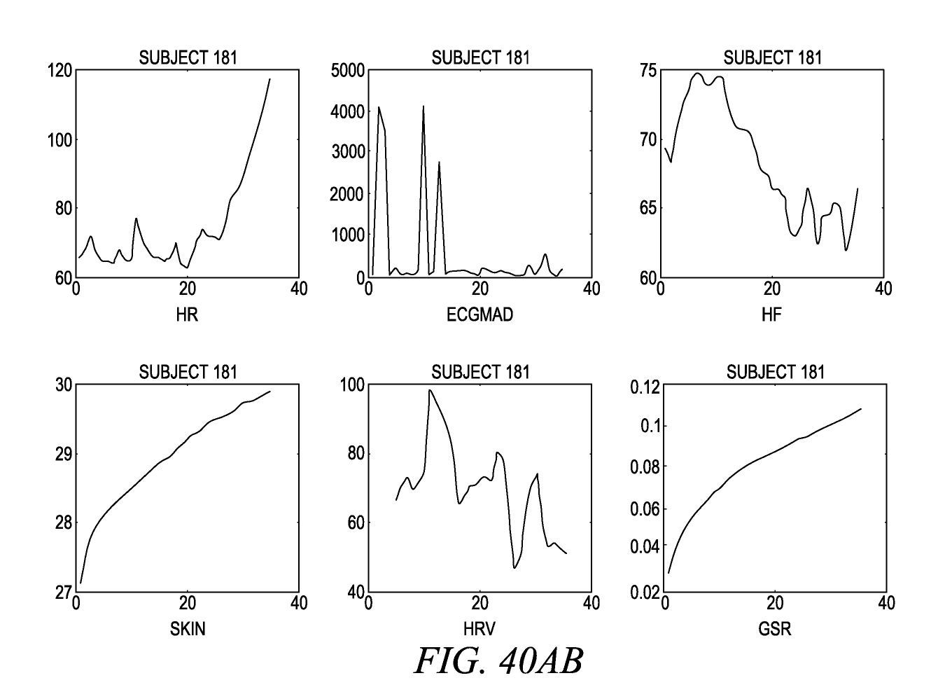

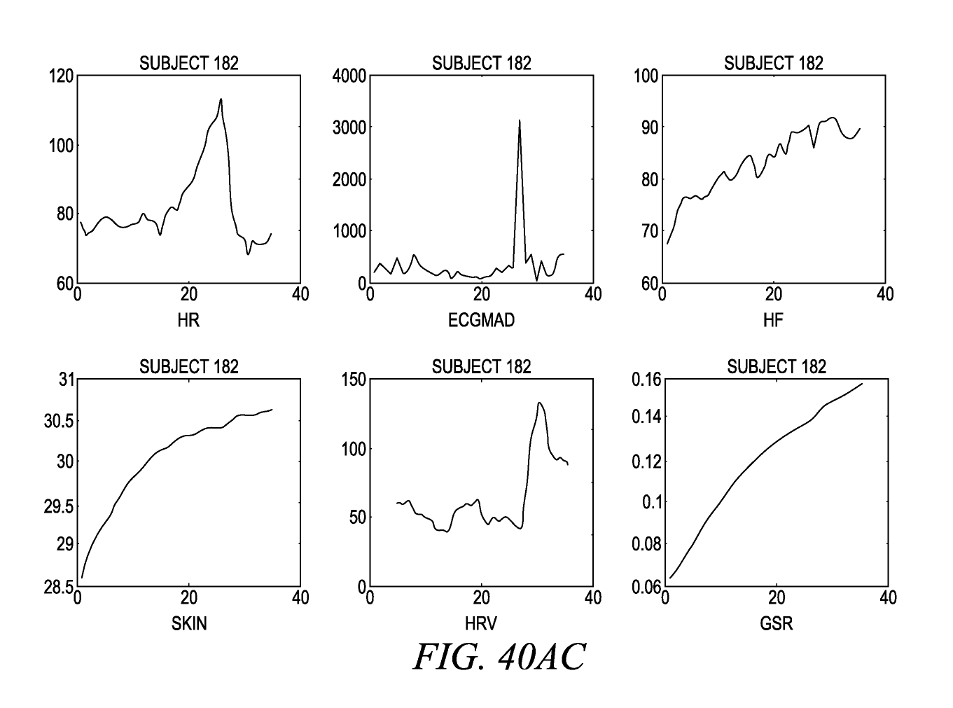

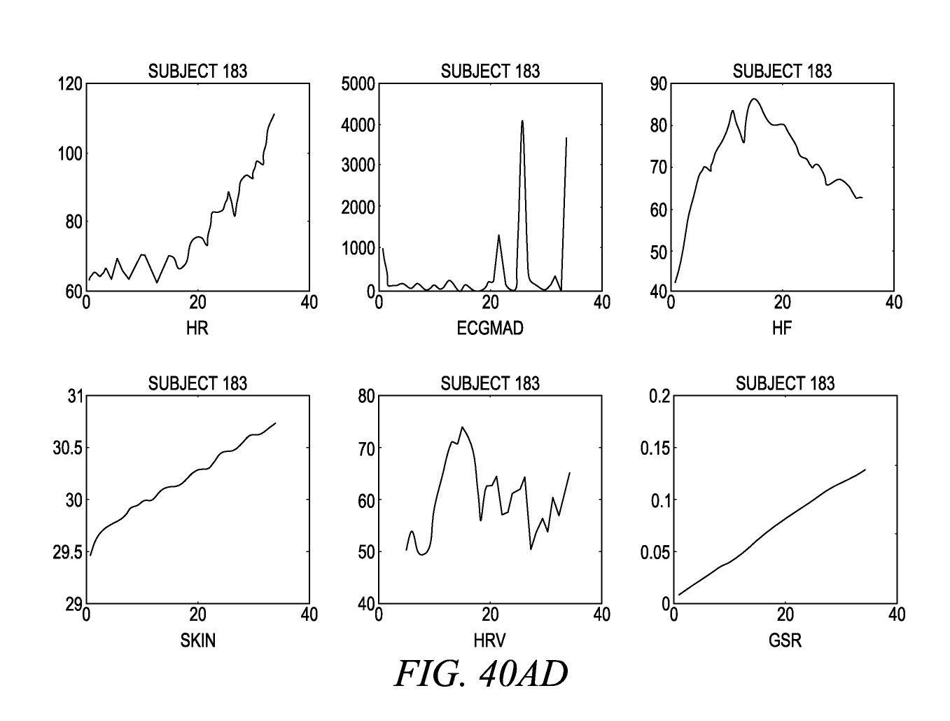

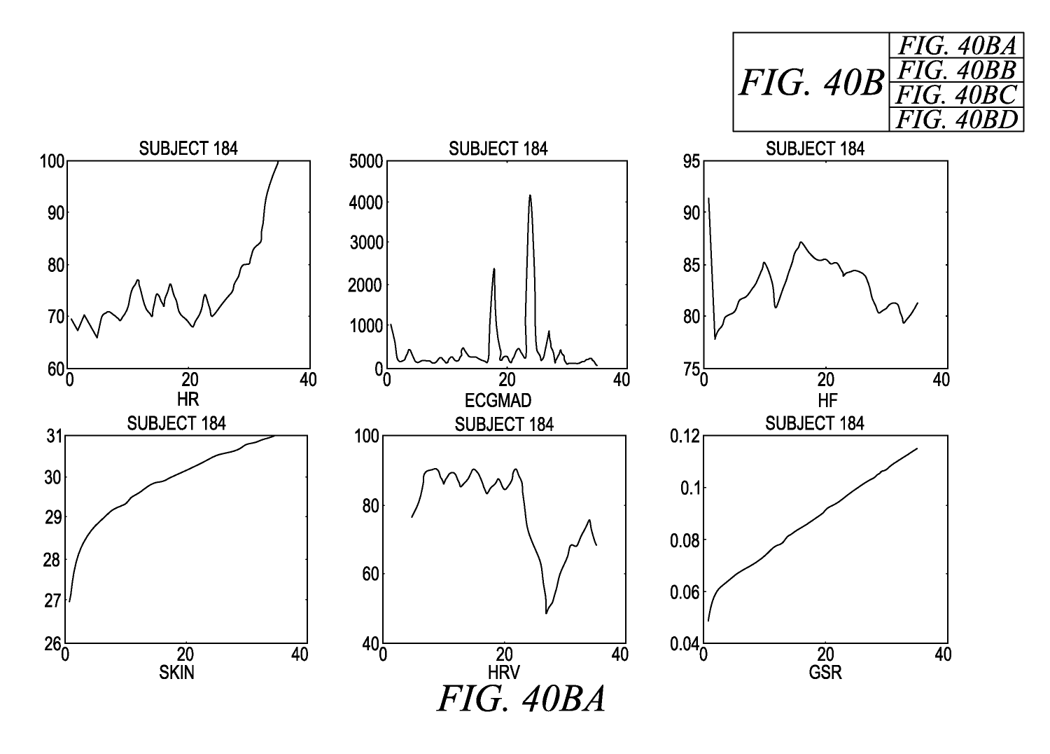

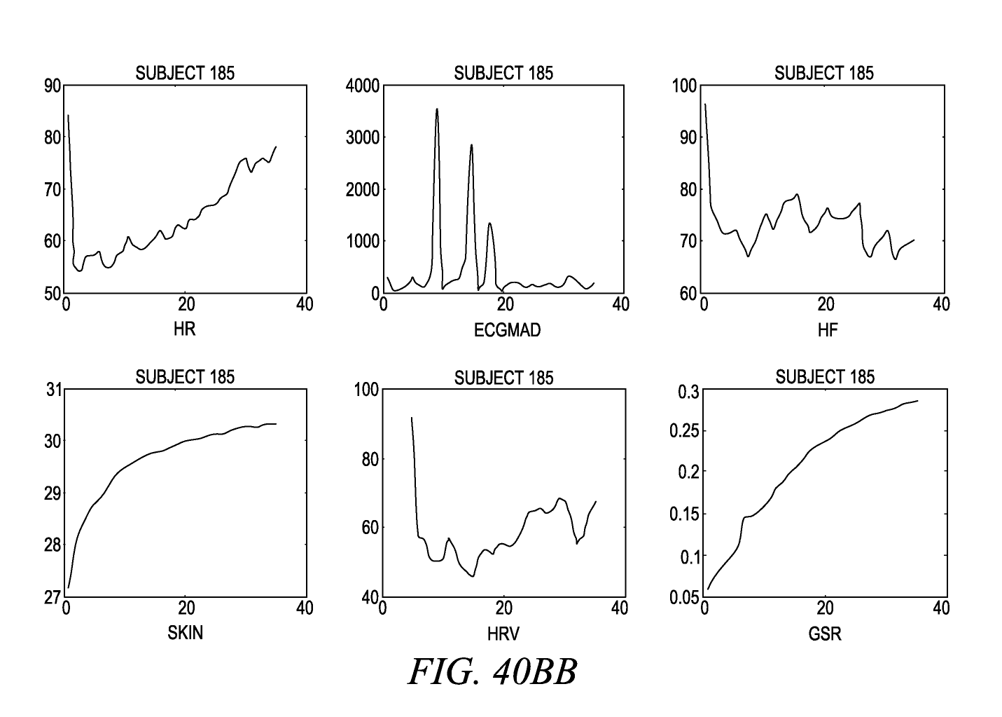

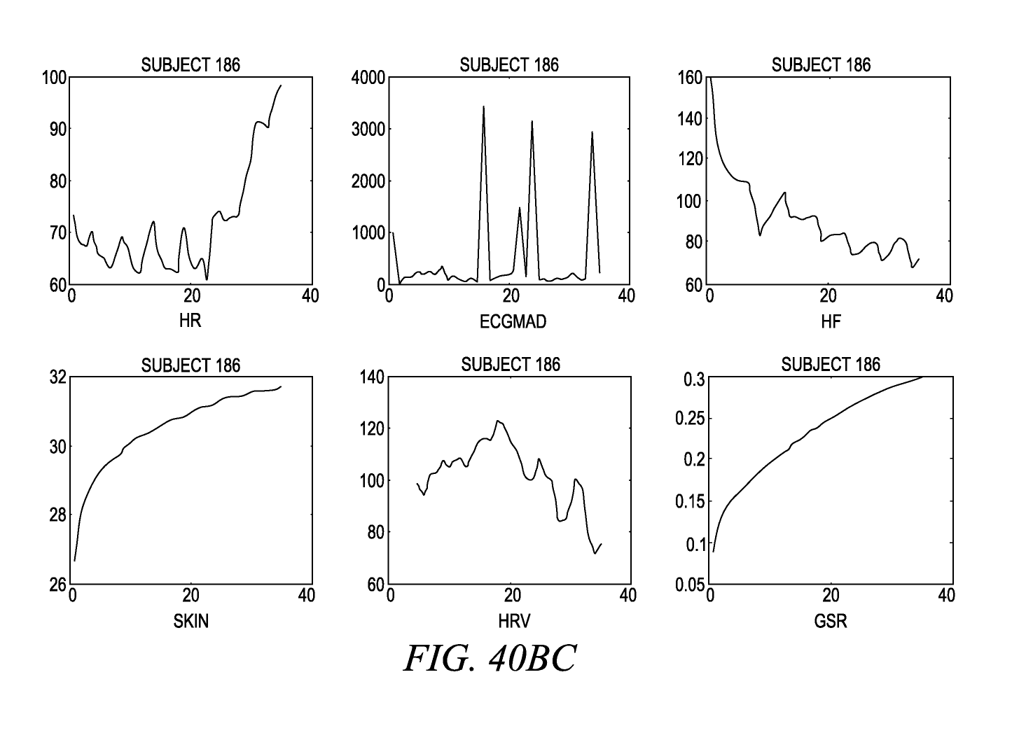

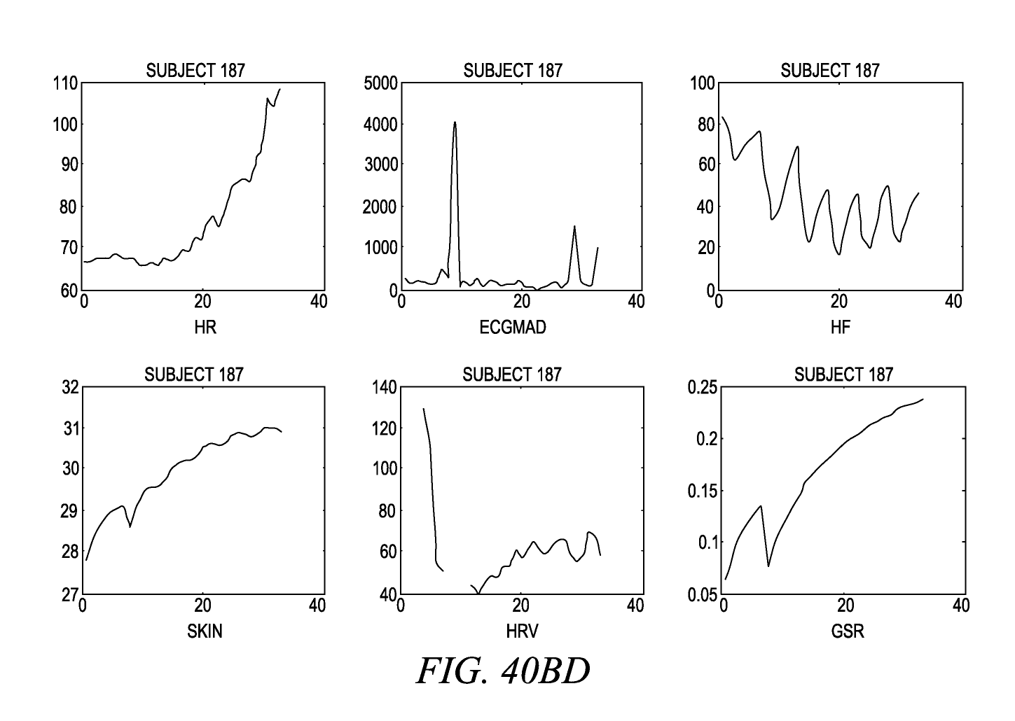

The following data as shown in FIGS. 40A-40H illustrates how the severity of LBNP (Lower Body Negative Pressure, described above) protocol (or exercise protocol) affect armband sensor values. For each plot, the X-axis represents severity stage: Stage 0 is a baseline stage, and the rest of the stages increase gradually in severity. The Y-axis in these graphs represents the units of the particular sensor mentioned in the graph. (For example, in the first graph of COVER (ambient temperature), the unit is in Celsius).

Each point in the graph is an average of all minutes under that particular stage averaged across all subjects (There are total 28 subjects who underwent the LBNP protocol, and there are total 14 subjects who participated in the exercise protocol). FIG. 40A is a measurement of ambiant temperature (COVER); FIG. 40B is a measure of galvanic skin response (GSR); FIG. 40C is a measure of heat flux (HF); FIG. 40D is a measure of heart rate (HR); FIG. 40E is a measure of heart rate variability; FIG. 40F is a measure of longitudinal accelerometer values averaged over each minute; FIG. 40G is a measure of longitudinal mean absolute difference values (as described in United States Patent Application 2007/0100666, the contents of which are herein incorporated by reference in their entirety); and FIG. 40H is a measure of energy expenditure (EE). The lines indicated by (-∘-) signify the average values of armband sensors for the exercise protocol with the lines indicated by (-▴-) signify the average values of armband sensors for the LBNP protocol. The final LBNP stage (stage 6) in the graphs closely mimics the effects of hemmorhagic shock.

### Example 2

The following data as illustrated in FIGS. 41A and 41B represents typical characteristics of the armband signals for the LBNP protocol. Each grid consists of 6 columns; each column representing an armband signal (From left to right—HR (Heart Rate), ECGMAD (Mean Absolute Difference of Raw ECG signal collected by the armband), HF (Heat Flux), SKIN Temperature; HR (Heart Rate Variability); and GSR (Galvanic Skin Response). Each row of the grid represents a particular subject. The first row has all the graphs for subject 180, the second row has all graphs for subject 181 and so on. The X-axis in each graph represents duration of the protocol which is roughly 40 minutes (each stage is roughly 5 minutes long, and the subject on average proceeds to stage 6—resulting in 30 min. on X axis+5 min. of baseline level+5 min. of recovery). The Y-axis is represents values of a corresponding unit of the armband variable in question (for example for SKIN—Y axis represents Celsius).

### Example 3

The classifier that detects hemorrhagic shock is designed in two levels. The first level distinguishes between LBNP and exercise. Once this distinction is made, the second level of classifier decides the severity of LBNP. Detecting a severe LBNP level is analogous to detecting a hemorrhagic shock.

For the first level of classifier: Energy expenditure, heart rate and GSR go up gradually in both the LBNP and exercise protocol as there is an increase in severity. However, accelerometer values behave differently for both the protocols. Even for supine and other low movement related exercises such as supine biking, on increased amount of motion is observed in the accelerometer variables, whereas during LBNP, the accelerometer variables remain static throughout the entire duration. This indicates a clear indication that EE, GSR, etc. are increasing despite a lack of motion.

Tables 6 and 7 illustrate the results of the classifier. These tables represent confusion matrices and accuracy statistics of the classifier models. Table 6 describes the results when the same set (of 14 users) is used for building the classifiers and then for testing. Table 7 consists of the results of leave-one out cross-validation. In this scheme: One user is kept out and the classifier model is built on the remaining users. Testing was performed on the user that was kept out. This procedure is repeated for all the users. This technique is more appropriate to measure the model's ability to generalize on unseen data.

TABLE 6
Train-test on the same dataset N = 14

| | Actual | |
|---|---|---|
| Predicted | Exercise | LBNP |
| Exercise | 420 | 20 |
| LBNP | 30 | 504 |
| Accuracy | 0.948665 | |
| Sensitivity/Recall/TP | 0.933333 | |
| Rate | | |
| Specificity/TN Rate | 0.961832 | |
| Precision | 0.954545 | 0.94382 |

TABLE 7
By Subject Cross-validation N = 14

| | Actual | |
|---|---|---|
| Predicted | Exercise | LBNP |
| Exercise | 405 | 35 |
| LBNP | 51 | 483 |
| Accuracy | 0.911704 | |
| Sensitivity/Recall/TP | 0.888158 | |
| Rate | | |
| Specificity/TN Rate | 0.932432 | |
| Precision | 0.920455 | 0.904494 |

The second level of classifier detects the severity of a LBNP level (given that that the event has been detected as an LBNP event using the first level of the classifier, that it is known beforehand that the protocol is an LBNP protocol). For this classifier, variables derived from heart rate, skin temperature, GSR and heat flux are useful. Tables 8 and 9 represent the confusion matrices and the accuracy statistics for the severity detection classifiers.

TABLE 8
Train-test on the same dataset N = 26

| | Actual | | |
|---|---|---|---|
| | Mild- | | |
| Predicted | Moderate | Severe | Average |
| Mild-Moderate | 492 | 51 | |
| Severe | 41 | 160 | |
| Accuracy | 0.876344086 | | |
| Recall | 0.923076923 | 0.758293839 | 0.840685 |
| Precision | 0.906077348 | 0.7960199 | 0.851049 |

TABLE 9
By Subject Cross-validation N = 26

| | Actual | | |
|---|---|---|---|
| | Mild- | | |
| Predicted | Moderate | Severe | Average |
| Mild-Moderate | 491 | 52 | |
| Severe | 44 | 157 | |
| Accuracy | 0.870967742 | | |
| Recall | 0.917757009 | 0.751196172 | 0.834477 |
| Precision | 0.904235727 | 0.781094527 | 0.842665 |

### Example 4

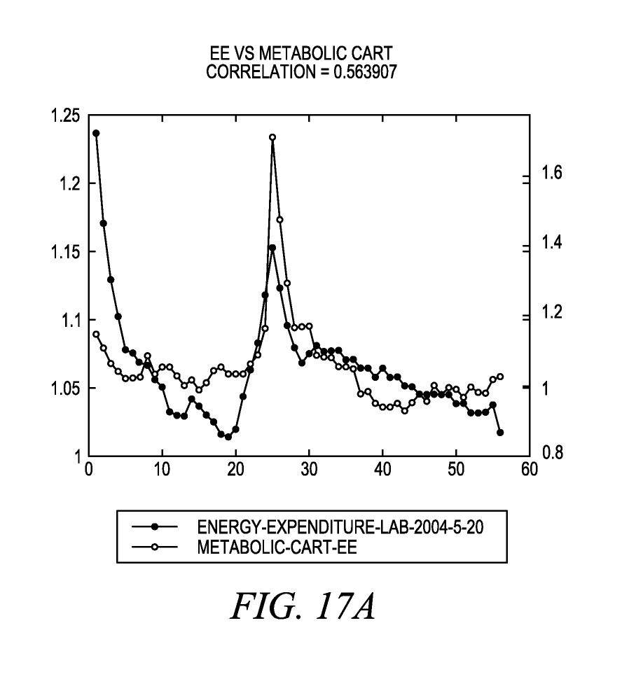

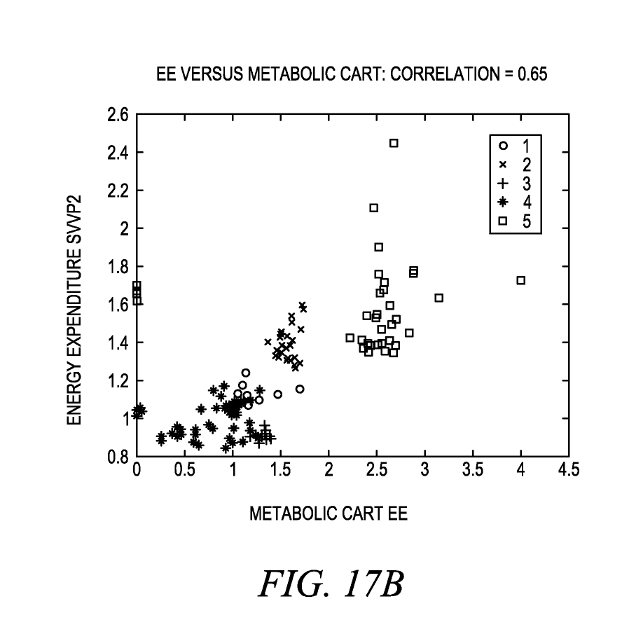

Preliminary data in 6 patients wearing the SenseWear Pro2 with the current sensors that did NOT include ECG and HeartBeat recognition demonstrate energy expenditure (EE) or oxygen consumption as measured by the armband correlated well with EE measured with a metabolic cart, as shown in FIGS. 17A and 17B. These results were obtained utilizing algorithms that were statistically developed for general, free-living, daily lifestyle application sets. FIG. 17A illustrates how effectively the calculation of estimated energy expenditure correlates with the true energy expenditure calculated by metabolic cart for one of the lab sessions while the subject is at rest. FIG. 17B depicts a scatter plot of measured energy expenditure versus estimated energy expenditure. The different scatter plot labels denote different subjects. As it can be seen, the algorithm is able to track energy expenditure impressively for all 5 subjects shown. This data has allowed the development of refined algorithms to address the under-estimation of the armband for this condition. Understanding the physiological condition of an injured subject prior to injury may have profound affects on data interpretation and clinical implications for treatment. For example, it is possible using the measure of oxygen consumption to determine oxygen debt which may have significant ability to predict outcome and thus provides a powerful means of triage; since oxygen debt has been one of the most indicative physiological variable to predict survival, survival with organ failure and death.

### Example 5

The SenseWear armband was used on subjects undergoing lower body negative pressure (LBNP). LBNP is used as a surrogate model of hemorrhage in order to examine the human physiological response to central volume loss as well as to develop new means of monitoring for remote triage and treatment of the wounded warfighter. In this model, conscious subjects are subjected to sequential timed increases in LBNP which finally result in a state of presyncope. During this time, a host of physiological variables are measured including continuous blood pressure and heart rate. The data from 6 subjects undergoing LBNP demonstrates first order proof of principle that the low level signals of GSR, temperature, and heat flux can be used to create algorithms that produce predicted shock index and pulse pressure values which closely track the values measured in real time. No heart rate monitoring was performed by the SenseWear armband.

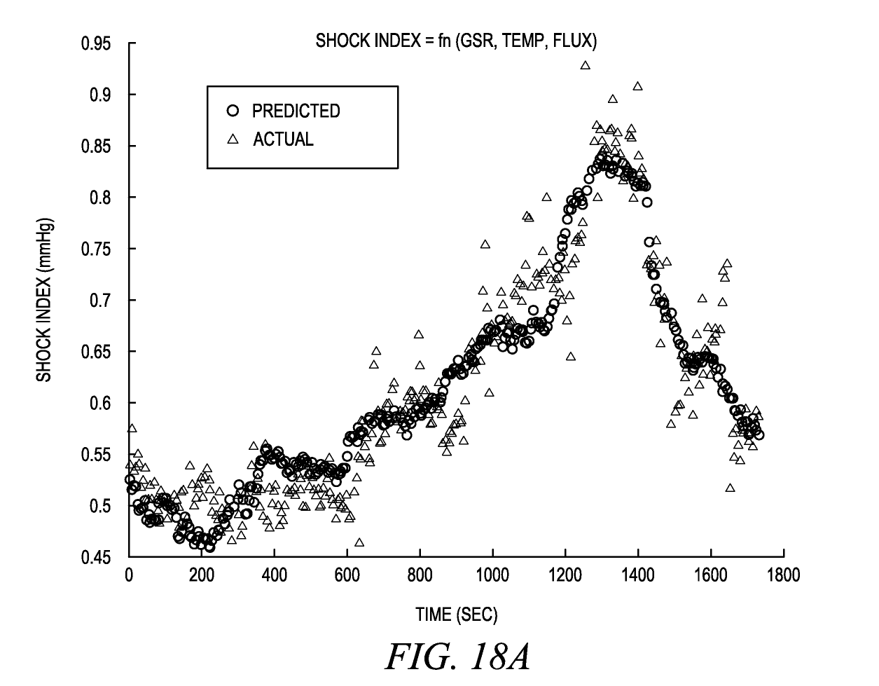

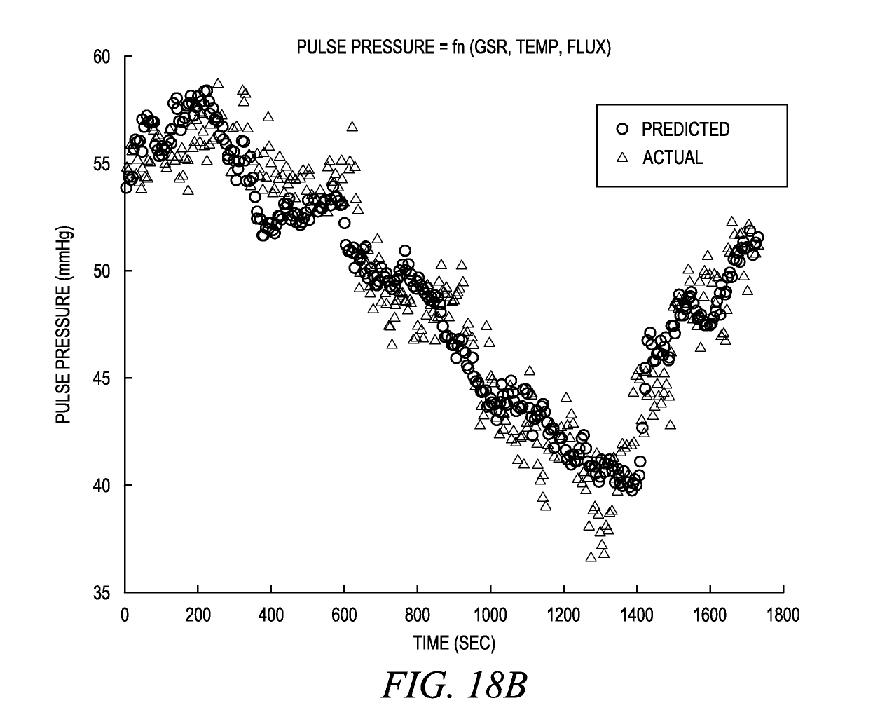

On the subjects studied, the algorithms were able to predict the shock index and pulse pressure with very high correlation and accuracy as illustrated in FIGS. 18A and 18B. These figures demonstrate the prediction performance on an “average subject”. Each point in the graph is the value of the variable averaged across all six users. The measured data points in the graph are actual) quantities of pulse pressure and shock index parameters, respectively for FIGS. 18A and B, averaged across all six users. The predicted pulse pressure and shock index values, respectively for 18A and 18B, are averaged across all six users.

The terms and expressions which have been employed herein are used as terms of description and not as limitation, and there is no intention in the use of such terms and expressions of excluding equivalents of the features shown and described or portions thereof, it being recognized that various modifications are possible within the scope of the invention claimed. Although particular embodiments of the present invention have been illustrated in the foregoing detailed description, it is to be further understood that the present invention is not to be limited to just the embodiments disclosed, but that they are capable of numerous rearrangements, modifications and substitutions.

What is claimed is:

1. A method for accurately deriving and reporting a critical care parameter of an individual comprising:
associating at least one physiological sensor with the body of said individual;
continuously collecting sensor output signals from said at least one physiological sensor for a period of time from said individual;
simultaneously collecting physiological data related to said critical care parameter of said individual;
applying at least one mathematical operation defining the association of said critical care parameter of said individual with said sensor output signals;
deriving values of said critical care parameter of said individual from said sensor output by applying said series of mathematical operations; and
reporting said critical care parameter as output.

2. The method of claim 1, wherein said mathematical operation is formed by:
modifying said present series of mathematical operations to form a modified series of mathematical operations based upon said derivation of said values of said a critical care parameter of said individual, such that said derived values of said a critical care parameter is consistently equivalent to said collected physiological data; and
deriving the values of said critical care parameter for said individual solely by applying said modified series of mathematical operations to said sensor output signals.

3. The method of claim 1, wherein said critical parameter is determined by a quantitative measurement of a physiological parameter.

4. The method of claim 1, wherein said critical care parameter is selected from the group consisting of oxygen hemorrhage (nontraumatic), traumatic hemorrhage, acute and chronic heart failure including myocardial infarction and acute arhythmias, cardiac arrest and cardiogenic shock, bacterial infection, viral infection, fungal infection, pneumonia, sepsis, septic shock, wounds, burns, hyper and hypothryoid, adrenal insufficiency, diabetic ketoacidosis, hyperthermia, hypothermia, preeclampsia, eclampsia, seizures, status epilepticus, drowning, acute respiratory failure, pulmonary embolism, traumatic brain injury, spinal cord injury, stroke, cerebral aneurysm; limb ischemia, coagulopathies, acute neuromuscular disease/failure, acute poisonings, vasoocclusive crisis and tumor lysis syndrome.

5. The method of claim 3, wherein the physiological parameter is selected from the group consisting of heart beat-to-beat variability, electrical activity of the heart over time, respiration rate, skin temperature, body core temperature, heat flow, galvanic skin response, electrical activity of muscles, bioimpedence, optical plethysmography, piezo motions, the spontaneous electrical activity of the brain, eye movement, blood pressure, body fat, activity, oxygen consumption, glucose level, carbon dioxide level, NADH level, tissue hemoglobin oxygen saturation level, body position, muscle pressure, UV radiation absorption, and lactate level.

6. The method of claim 3, wherein the physiological parameter is determined by a method selected from the group consisting of measuring heart rate, skin surface potential, chest volume change, surface temperature probe, esophageal or rectal probe, heat flux, skin conductance, skin surface potentials eye movement, non-invasive Korotkuff sounds, body impedance, body movement, body impedance, body movement, oxygen uptake, electrochemical measurement, optical spectroscopy, fluorescence spectroscopy, mercury switch array, think film piezoelectric sensors, UV sensitive photo cells.

7. The method of claim 1 wherein said critical care parameter is oxygen consumption.

8. The method of claim 1 wherein said critical parameter is oxygen debt.

9. A system for accurately deriving and reporting a critical care parameter of an individual comprising:
at least one physiological sensor associated with the body of said individual generating sensor output signals;
a memory circuit containing stored mathematical operations for the identification of a critical care parameter of said individual from said sensor output signals;
a processor in electronic communication with said sensors and said memory circuit for: (i) receiving said sensor output signals from said at least one sensor, and (ii) applying said stored mathematical operations to said sensor output signals to derive said a critical care parameter of said individual; and
a display, in electronic communication with said processor for displaying the derived quantitative critical care parameter for said individual.

10. The system of claim 9, wherein the memory circuit further comprises collected sensor output signals relating to measured physiological data

11. The system of claim 9, wherein said processor modifies said mathematical operations in accordance with said derivation of said values of said quantitative a critical care parameter of said individual such that such that said modified series of mathematical operations are consistently equivalent to said collected physiological data within a defined tolerance range.

12. The system of claim 9, wherein said critical parameter is determined by a quantitative measurement of a physiological parameter.

13. The system of claim 9, wherein said critical care parameter is selected from the group consisting of hemorrhage (nontraumatic), traumatic hemorrhage, acute and chronic heart failure including myocardial infarction and acute arhythmias, cardiac arrest and cardiogenic shock, bacterial infection, viral infection, fungal infection, pneumonia, sepsis, septic shock, wounds, burns, hyper and hypothryoid, adrenal insufficiency, diabetic ketoacidosis, hyperthermia, hypothermia, preeclampsia, eclampsia, seizures, status epilepticus, drowning, acute respiratory failure, pulmonary embolism, traumatic brain injury, spinal cord injury, stroke, cerebral aneurysm; limb ischemia, coagulopathies, acute neuromuscular disease/failure, acute poisonings, vasoocclusive crisis and tumor lysis syndrome.

14. The system of claim 12, wherein the physiological parameter is selected from the group consisting of heart beat-to-beat variability, electrical activity of the heart over time, respiration rate, skin temperature, body core temperature, heat flow, galvanic skin response, electrical activity of muscles, bioimpedence, optical plethysmography, piezo motions, the spontaneous electrical activity of the brain, eye movement, blood pressure, body fat, activity, oxygen consumption, glucose level, carbon dioxide level, NADH level, tissue hemoglobin oxygen saturation level, body position, muscle pressure, UV radiation absorption, and lactate level.

15. The system of claim 12, wherein the physiological parameter is determined by a method selected from the group consisting of measuring heart rate, skin surface potential, chest volume change, surface temperature probe, esophageal or rectal probe, heat flux, skin conductance, skin surface potentials (EMG, EEG), eye movement, non-invasive Korotkuff sounds, body impedance, body movement, oxygen uptake, electrochemical measurement, optical spectroscopy, fluorescence spectroscopy, mercury switch array, think film piezoelectric sensors, UV sensitive photo cells,

16. The system of claim 9 wherein said critical care parameter is oxygen consumption.

17. The system of claim 9 wherein said critical parameter is oxygen debt.

18. A device for accurately deriving and reporting a critical care parameter of an individual comprising:
at least one physiological sensor associated with the body of said individual generating sensor output signals;
a memory circuit containing stored mathematical operations for the derivation of a quantitative a critical care parameter of said individual from said sensor output signals;
a processor in electronic communication with said sensors and said memory circuit for: (i) receiving said sensor output signals from said at least one sensor and (ii) applying said stored mathematical operations to said sensor output signals to derive said critical care parameter; and
a display, in electronic communication with said processor for displaying the derived quantitative critical care parameter for said individual.

19. The device of claim 18, wherein said critical parameter is determined by a quantitative measurement of a physiological parameter.

20. The device of claim 18, wherein said critical care parameter is selected from the group consisting of hemorrhage (nontraumatic), traumatic hemorrhage, acute and chronic heart failure including myocardial infarction and acute arhythmias, cardiac arrest and cardiogenic shock, bacterial infection, viral infection, fungal infection, pneumonia, sepsis, septic shock, wounds, burns, hyper and hypothryoid, adrenal insufficiency, diabetic ketoacidosis, hyperthermia, hypothermia, preeclampsia, eclampsia, seizures, status epilepticus, drowning, acute respiratory failure, pulmonary embolism, traumatic brain injury, spinal cord injury, stroke, cerebral aneurysm; limb ischemia, coagulopathies, acute neuromuscular disease/failure, acute poisonings, vasoocclusive crisis and tumor lysis syndrome.

21. The device of claim 19, wherein the physiological parameter is selected from the group consisting of heart beat-to-beat variability, electrical activity of the heart over time, respiration rate, skin temperature, body core temperature, heat flow, galvanic skin response, electrical activity of muscles, bioimpedence, optical plethysmography, piezo motions, the spontaneous electrical activity of the brain, eye movement, blood pressure, body fat, activity, oxygen consumption, glucose level, carbon dioxide level, NADH level, tissue hemoglobin oxygen saturation level, body position, muscle pressure, UV radiation absorption, and lactate level.

22. The device of claim 19, wherein the physiological parameter is determined by a method selected from the group consisting of measuring heart rate, skin surface potential, chest volume change, surface temperature probe, esophageal or rectal probe, heat flux, skin conductance, skin surface potentials (EMG, EEG), eye movement, non-invasive Korotkuff sounds, body impedance, body movement, oxygen uptake, electrochemical measurement, optical spectroscopy, fluorescence spectroscopy, mercury switch array, think film piezoelectric sensors, UV sensitive photo cells.

23. The device of claim 18 wherein said critical care parameter is oxygen consumption.

24. The device of claim 18 wherein said critical care parameter is oxygen debt.

25. A system for determining a critical care parameter, comprising:
a. a wearable sensor device comprising at least one non-invasive sensor for generating a sensor output signal;
b. a memory circuit containing stored instructions that when executed derive a critical care parameter of said individual from said sensor output signal; and
c. a processor in electronic communication with said sensors and said memory circuit for: (i) receiving said sensor output signal from said noninvasive sensor, and (ii) applying said stored instructions to derive said a critical care parameter of said individual.

26. The system of claim 25 wherein said non-invasive sensor is a galvanic skin response sensor.

27. The system of claim 25 wherein said sensor generates data indicative of a heart related parameter.

28. The system of claim 25 further comprising an additional sensor generating a sensor output signal.

29. The system of claim 28 wherein said memory circuit comprises additional stored instructions when executed also derive a context of said individual and utilize said context in deriving said critical care parameter; and
wherein said processor is further for (i) receiving said additional sensor output signal, (ii) applying said additional instructions to determine said context, (iii) utilizing said context to derive said critical care parameter.

30. The system of claim 29 wherein said context is that the individual is substantially sedentary.
